(19) United States
(12) Patent Application Publication
(10) Pub. No.: US 2020/0340013 A1
(43) Pub. Date: Oct. 29, 2020

(54) METHODS AND COMPOSITIONS FOR INHIBITION OF INNATE IMMUNE RESPONSE ASSOCIATED WITH AAV TRANSDUCTION

(71) Applicant: The University of North Carolina at Chapel Hill, Chapel Hill, NC (US)

(72) Inventors: Chengwen Li, Chapel Hill, NC (US); Richard Jude Samulski, Chapel Hill, NC (US)

(21) Appl. No.: 16/963,023

(22) PCT Filed: Jan. 18, 2019
(86) PCT No.: PCT/US2019/014211

§ 371 Date: Jul. 17, 2020

Related U.S. Application Data
(60) Provisional application No. 62/619,468, filed on Jan. 19, 2018.

(51) Int. Cl. C12N 15/86; C12N 7/00; C12N 15/113; A61K 48/00; A61K 35/76; A61K 31/52

(52) U.S. Cl. CPC C12N 15/86; C12N 7/00; C12N 15/113; A61K 48/0008; C12N 2800/10; A61K 31/52; C12N 2750/14143; C12N 2310/141; A61K 35/76

(57) ABSTRACT

Disclosed herein are methods and compositions for inhibition of an innate immune response associated with AAV transduction.

### STATEMENT OF PRIORITY

This application claims the benefit, under 35 U.S.C. § 119(e), of U.S. Provisional Application Ser. No. 62/619,468, filed Jan. 19, 2018, the entire contents of which are incorporated by reference herein.

### STATEMENT OF GOVERNMENT SUPPORT

This invention was made with government support under Grant Numbers AI117408, HL125749, AI072176 and AR064369, awarded by the National Institutes of Health. The government has certain rights in the invention.

### STATEMENT REGARDING ELECTRONIC FILING OF A SEQUENCE LISTING

A Sequence Listing in ASCII text format, submitted under 37 C.F.R. § 1.821, entitled 5470-819WO_ST25.txt, 7,205 bytes in size, generated on Jan. 18, 2019 and filed via EFS-Web, is provided in lieu of a paper copy. This Sequence Listing is hereby incorporated herein by reference into the specification for its disclosures.

### FIELD OF THE INVENTION

This invention is directed to methods and compositions for inhibition of an innate immune response associated with AAV transduction.

### BACKGROUND OF THE INVENTION

Adeno-associated virus (AAV) vectors have been successfully applied in clinical trials in patients with hemophilia and blindness disorders. In some patients with hemophilia B, after delivery of an AAV vector encoding factor IX (FIX), transgene expression was decreased with elevated liver enzymes at weeks 6 to 10. Administration of prednisone prevented the FIX decrease and increased FIX to previous levels in the blood. This phenomenon was never observed in pre-clinical trials in rodents and large animals. Capsid specific cytotoxic T lymphocytes (CTLs) were detected in these patients; therefore, it has been suggested that the therapeutic failure results from the clearance of AAV transduced hepatocytes mediated by capsid specific CTLs. This presumption is not fully supported by the present invention.

First of all, kinetics study of AAV capsid antigen presentation showed that efficient antigen presentation occurs immediately after AAV administration and gradually decreases to undetected levels at later time points post AAV transduction. This implicates that capsid specific CTLs should kill most AAV transduced cells at the early time points, but could not impact transgene expression at a later time. Secondly, if there was a CTL mediated elimination of AAV transduced target cells, the administration of prednisone would not restore the transgene expression to previous levels. Thirdly, no FIX expression was inhibited although capsid specific CTL response was observed in some patients. Therefore, other mechanisms may play a role in the FIX decrease after AAV gene delivery. It has been demonstrated that an innate immune response is immediately activated following AAV administration via TLR9 and TLR2 recognition; however, there are no studies about innate immune response induction at later time points after AAV administration or its role in transgene expression.

AAV is a single-stranded DNA virus. Its genome comprises the rep and cap sequences flanked by two inverted terminal repeats (ITR). Replacement of the rep and the cap genes with a therapeutic cassette (comprising a promoter, one or more therapeutic transgene and a poly(A) (“pA”) tail) results in an AAV vector construct. The AAV ITR has been shown to have a promoter function, which implicates that the plus strand RNA transcribed from the 5′ ITR and the minus strand RNA transcribed from the 3′ ITR could be generated in AAV transduced cells. This assumption was supported by findings described herein, wherein transgene expression was increased when a plasmid with the 3′-ITR was deleted via transfection analysis (FIG. 9). The minus strand of RNA transcribed by the 3′-ITR promoter might serve as antisense RNA to knock down transgene expression. The plus strand RNA and minus strand RNA generated from the AAV ITR promoters on both terminals are able to anneal and form a dsRNA in the cytoplasm of AAV transduced cells. Additionally, it has been shown that some promoters for gene delivery have bi-directional transcription function to generate minus strand RNA, by which is also possible to form a dsRNA. A third possibility to form dsRNA from gene delivery is the secondary structure formation of mRNA from a transgene cassette due to modification of transgene cDNA sequences. This dsRNA formation potentially activates the innate immune response.

MDA5 and RIG-I are cytoplasmic viral RNA sensors capable of activating type I interferon signaling pathways after virus infection, so they play a critical role in antiviral innate immunity. MDA5 and RIG-I share high sequence similarity and a common signaling adaptor, mitochondrial antiviral signaling (MAVS), but they play non-redundant functions in antiviral immunity by recognizing different viruses or viral RNA. RIG-I recognizes 5′-triphosphorylated (PPP) blunt-ended double-stranded RNA (dsRNA) or single-stranded RNA hairpins that are often present in a variety of positive and negative strand viruses. MDA5 recognizes relatively long dsRNA in the genome of dsRNA viruses or dsRNA replication intermediates of positive-strand viruses, such as encephalomyocarditis virus (EMCV) and poliovirus.

The present invention overcomes previous shortcomings in the art by providing compositions and methods of their use in inhibiting an innate immune response associated with AAV transduction in a subject.

### SUMMARY OF THE INVENTION

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In one embodiment, the present invention provides a recombinant nucleic acid molecule, comprising an adeno-associated virus (AAV) 5′ inverted terminal repeat (ITR), a nucleotide sequence of interest (NOI) operably associated with a promoter and an AAV 3′ ITR, wherein the recombinant nucleic acid molecule further comprises: a) a poly(A) (pA) sequence downstream of the 5′ ITR and upstream of the promoter, in 3′ to 5′ orientation and a pA sequence upstream of the 3′ ITR and downstream of the NOI, in 3′ to 5′ orientation; b) a pA sequence upstream of the 3′ ITR and downstream of the NOI, in 3′ to 5′ orientation; c) a first pA sequence upstream of the 3′ ITR and downstream of the NOI, in 3′ to 5′ orientation and a second pA sequence downstream of the first pA sequence and upstream of the 3′ ITR, in a 5′ to 3′ orientation; d) a first pA sequence upstream of the 3′ ITR and downstream of the NOI, in 3′ to 5′ orientation and a second pA sequence downstream of the NOI and upstream of the first pA, in a 5′ to 3′ orientation; e) a first pA sequence upstream of the 3′ ITR and downstream of the NOI, in 3′ to 5′ orientation and a second pA sequence downstream of the 5′ ITR and upstream of the promoter, in a 5′ to 3′ orientation; f) a first pA sequence downstream of the 5′ ITR and upstream of the promoter, in 3′ to 5′ orientation, a second pA sequence downstream of the NOI and upstream of a third pA sequence, in 5′ to 3′ orientation and the third pA sequence downstream of the second pA sequence and upstream of the 3′ ITR, in 3′ to 5′ orientation; g) a first pA sequence downstream of the 5′ ITR and upstream of the promoter, in 5′ to 3′ orientation, a second pA sequence downstream of the NOI and upstream of a third pA sequence, in 3′ to 5′ orientation and the third pA sequence downstream of the second pA sequence and upstream of the 3′ ITR, in 5′ to 3′ orientation; h) a first pA sequence downstream of the 5′ ITR and upstream of the promoter, in 5′ to 3′ orientation, a second pA sequence downstream of the NOI and upstream of a third pA sequence, in 5′ to 3′ orientation and the third pA sequence downstream of the second pA sequence and upstream of the 3′ ITR, in 3′ to 5′ orientation; i) a first pA sequence downstream of the 5′ ITR and upstream of the promoter, in 5′ to 3′ orientation, a second pA sequence downstream of the NOI and upstream of a third pA sequence, in 3′ to 5′ orientation and the third pA sequence downstream of the second pA sequence and upstream of the 3′ ITR, in 5′ to 3′ orientation; j) a first pA sequence downstream of the 5′ ITR and upstream of a second pA sequence, in 3′ to 5′ orientation, the second pA sequence downstream of the first pA sequence and upstream of the promoter, in 5′ to 3′ orientation; a third pA sequence downstream of the NOI and upstream of a fourth pA sequence, in 5′ to 3′ orientation and the fourth pA sequence downstream of the third pA sequence and upstream of the 3′ ITR, in 3′ to 5′ orientation; k) a first pA sequence downstream of the 5′ ITR and upstream of a second pA sequence, in 3′ to 5′ orientation, the second pA sequence downstream of the first pA sequence and upstream of the promoter, in 5′ to 3′ orientation; a third pA sequence downstream of the NOI and upstream of a fourth pA sequence, in 3′ to 5′ orientation and the fourth pA sequence downstream of the third pA sequence and upstream of the 3′ ITR, in 5′ to 3′ orientation; 1) a first pA sequence downstream of the 5′ ITR and upstream of a second pA sequence, in 5′ to 3′ orientation, the second pA sequence downstream of the first pA sequence and upstream of the promoter, in 3′ to 5′ orientation; a third pA sequence downstream of the NOI and upstream of a fourth pA sequence, in 5′ to 3′ orientation and the fourth pA sequence downstream of the third pA sequence and upstream of the 3′ ITR, in 3′ to 5′ orientation; and/or m) a first pA sequence downstream of the 5′ ITR and upstream of a second pA sequence, in 5′ to 3′ orientation, the second pA sequence downstream of the first pA sequence and upstream of the promoter, in 3′ to 5′ orientation; a third pA sequence downstream of the NOI and upstream of a fourth pA sequence, in 3′ to 5′ orientation and the fourth pA sequence downstream of the third pA sequence and upstream of the 3′ ITR, in 5′ to 3′ orientation.

In another embodiment, the present invention provides a recombinant nucleic acid molecule, comprising an adeno-associated virus (AAV) vector cassette of a first AAV serotype, comprising an AAV 5′ inverted terminal repeat (ITR), a nucleotide sequence of interest (NOI) operably associated with a promoter and an AAV 3′ ITR, wherein the recombinant nucleic acid molecule comprises an AAV 5′ ITR and/or an AAV 3′ ITR from a second AAV serotype that is different than the first AAV serotype and replaces the 5′ ITR and/or 3′ ITR of the first AAV serotype and in particular embodiments, wherein the ITR of second AAV serotype has no promoter function or reduced promoter function as compared with the promoter function of the ITR of the first AAV serotype. In this embodiment, the first AAV serotype can be any AAV serotype now known or later identified and the second AAV serotype that is different that the first AAV serotype can be any AAV serotype now known or later identified. In some embodiments, the first AAV serotype is AAV2 and the ITR of the second AAV serotype is AAV5. For example, the recombinant nucleic acid molecule can comprise an AAV vector cassette of AAV2, said cassette of AAV2 comprising a 5′ and/or 3′ ITR of AAV5.

In a further embodiment, the present invention provides a recombinant nucleic acid molecule, comprising an adeno-associated virus (AAV) 5′ inverted terminal repeat (ITR), a nucleotide sequence of interest (NOI) operably associated with a promoter and an AAV 3′ ITR, wherein the 5′ ITR and/or the 3′ ITR that is modified (e.g., by substitution, insertion and/or deletion) to diminish or eliminate promoter activity from the 5′ ITR and/or the 3′ ITR.

In another embodiment, the present invention provides a recombinant nucleic acid molecule, comprising an AAV 5′ ITR, an NOI operably associated with a promoter, a pA sequence in 3′ to 5′ orientation and an AAV 3′ ITR, wherein the NOI sequence is fused with (e.g., in frame with; upstream and/or downstream of) one or more than one nucleotide sequence that encodes an interfering RNA sequence that targets one or more than one cytoplasmic dsRNA sensor.

In some embodiments, the present invention provides A) a recombinant nucleic acid molecule, comprising an AAV 5′ ITR, an NOI operably associated with a first promoter, a first pA sequence in 3′ to 5′ orientation, a nucleotide sequence that encodes an interfering RNA sequence that targets a cytoplasmic dsRNA sensor, operably associated with a second promoter, a second pA sequence and an AAV 3′ ITR; B) A recombinant nucleic acid molecule, comprising an AAV 5′ ITR, a NOI operably associated with a first promoter, a pA sequence in 3′ to 5′ orientation, a short hairpin RNA (shRNA) sequence that targets a cytoplasmic dsRNA sensor, operably associated with a second promoter, and an AAV 3′ ITR; C) a recombinant nucleic acid molecule, comprising an AAV 5′ ITR, a shRNA that targets a cytoplasmic dsRNA sensor, operably associated with a first promoter, a NOI operably associated with a second promoter, a pA sequence in 3′ to 5′ orientation and an AAV 3′ ITR; D) a recombinant nucleic acid molecule, comprising, in the following order: an AAV 5′ ITR, a NOI and a micro RNA (miRNA) sequence that targets a cytoplasmic dsRNA sensor, both operably associated with a promoter, a pA sequence in 3′ to 5′ orientation, and an AAV 3′ ITR; E) A recombinant nucleic acid molecule, comprising, in the following order; an AAV 5′ ITR, a miRNA that targets a cytoplasmic dsRNA sensor and a NOI, both operably associated with a promoter, a pA sequence in 3′ to 5′ orientation, and an AAV 3′ ITR; and/or E) a recombinant nucleic acid molecule, comprising, in the following order: an AAV 5′ ITR, a NOI comprising a miRNA intron sequence within the NOI, the NOI being operably associated with a promoter, a pA sequence in 3′ to 5′ orientation, and an AAV 3′ ITR.

Another aspect of the invention relates to a rAAV vector genome comprising the recombinant nucleic acid molecule described above. Another aspect of the invention relates to an AAV particle comprising the rAAV genome that comprises the nucleic acid molecule described above. Another aspect of the invention relates to a composition comprising the rAAV particle.

Further provided herein is a composition comprising a first recombinant nucleic acid molecule comprising an AAV 5′ ITR, a NOI operably associated with a promoter, a pA sequence in 3′ to 5′ orientation, and an AAV 3′ ITR and a second recombinant nucleic acid molecule comprising an interfering RNA sequence that targets a cytoplasmic dsRNA sensor.

Nonlimiting examples of a cytoplasmic dsRNA of this invention include MDA5, MAVS, RIG-1, TRAF6, TRAF5, RIP1, FADD, IRF, TRAF3, NAP1, TBK1, IKK, IκB, TANK and any other molecules involved in MAVS downstream signaling, in any combination and order in a recombinant nucleic acid molecule of this invention.

Nonlimiting examples of an interfering RNA (RNAi) of this invention include small interfering RNA (siRNA), short hairpin RNA (shRNA), microRNA (miRNA), long double stranded RNA (long dsRNA), antisense RNA, ribozymes, etc., as are known in the art, as well as any other interfering RNA or inhibitory RNA now known or later identified.

The present invention further provides a recombinant nucleic acid molecule, comprising an AAV 5′ ITR, an NOI and an inhibitor of MAVS signaling, both operably associated with a promoter, a pA sequence in 3′ to 5′ orientation and an AAV 3′ ITR.

Also provided herein is a recombinant nucleic acid molecule, comprising an AAV 5′ ITR, a NOT operably associated with a first promoter, a first pA sequence in 3′ to 5′ orientation, an inhibitor of MAVS signaling operably associated with a second promoter, a second pA sequence in 3′ to 5′ orientation and an AAV 3′ ITR.

In additional embodiments, the present invention provides a composition comprising a first recombinant nucleic acid molecule comprising an AAV 5′ ITR, a NOI operably associated with a promoter, a pA sequence in 3′ to 5′ orientation, and an AAV 3′ ITR and a second recombinant nucleic acid molecule comprising an inhibitor of MAVS signaling and a pA sequence in 3′ to 5′ orientation.

Nonlimiting examples of an inhibitor of MAVS signaling include a serine protease NS3-4A from hepatitis C virus, proteases from Hepatitis A virus and GB virus B, and hepatitis B virus (HBV) X protein, poly(rC)-binding protein 2, the 20S proteasomal subunit PSMA7, and mitofusin 2, as well as any other inhibitor of MAVS signaling now known or later identified.

A method is also provided herein, of enhancing transduction of an AAV vector in cells of a subject, comprising administering to the subject an AAV vector and an agent that interferes with dsRNA activation pathways in cells of the subject.

Nonlimiting examples of an agent that interferes with dsRNA activation pathways include 2-aminopurine, a steroid (e.g., hydrocortisone as shown in FIGS. 28 and 29), and any other agent that interferes with dsRNA activation pathways in a cell as now known or later identified.

In some embodiments, the AAV vector and the agent(s) of this invention can be administered to the subject simultaneously and/or subsequently, in any order and in any time interval (e.g., hours, days, weeks, etc.)

### BRIEF DESCRIPTION OF THE DRAWINGS

FIGS. 1A and 1B show IFN-β inhibited AAV transgene expression in the HeLa cell line. HeLa cells were transduced with 5×10³ particles of AAV2/luciferase per cell. (1A) After 24 h, recombinant human IFN-β was added to the medium at a different dose. Transgene expression was detected by luciferase assay at day 1, 2, 4 and 6 after supplementation of IFN-β. (1B) Recombinant human IFN-β was added to the medium every day at 0.5 ng/mL. Transgene expression was detected by luciferase assay at day 1, 2, 4 and 6. ***p<0.001, when compared to no IFN-β treatment.

FIG. 2 shows Poly(I:C) inhibited AAV transgene expression in cell lines. HeLa or Huh7 cells were transduced with 5×10³ particles of AAV2/luciferase per cell. 2 μg/mL poly(I:C) was added at different time points: 18 h before AAV transduction, day 0 or day 3. Luciferase expression was detected 3 days after poly(I:C) transfection.

FIGS. 3A-3E show that the dsRNA immune response is activated at a later time point after AAV transduction. HeLa cells were transduced with 5×10³ particles of dsAAV2/GFP per cell. The expression of MDA5 (3A), RIG-I (3B) and IFN-β (3C) in HeLa cells was detected by Q-PCR at different time points after transduction. *p<0.05, **p<0.01, when compared to the PBS group. The data represents the average and standard deviation from 3 experiments. For each experiment, PBS or AAV infected group contain 2 or 3 wells of cells. For Q-PCR data analysis, one sample from PBS group was normalized to 1 in each timepoint of each experiment. MDA5 expression in HeLa cells in each group were detected by western blot 8 days after dsAAV2/GFP transduction (3D). The relative level of MDA5 expression was calculated based on the intensity of β-actin protein (3E). ***p<0.001, when compared to the PBS group.

FIGS. 4A and 4B show the dsRNA response profile in different cell lines. (4A) Huh7, HEK293 and HepG2 cells were transduced with 5×10³ particles of AAV2/GFP per cell. The expression of MDA5, RIG-I and IFN-β was detected by Q-PCR at day 7. *p<0.05, **p<0.01, when compared to the PBS group. (4B) AAV2 with different transgenes was added to HeLa cells with 5×10³ particles per cell. The expression of MDA5, RIG-I and IFN-β was detected by Q-PCR at day 7 after AAV transduction. For Q-PCR data analysis, samples from PBS group were normalized to 1 in each experiment.

FIGS. 5A and 5B show the dsRNA innate immune response in human primary hepatocytes after dsAAV2/GFP transduction. Fresh human primary hepatocytes from 12 individuals were transduced by AAV2/GFP with 5×10³ particles per cell. The expression of MDA5, RIG-I and IFN-β was detected by Q-PCR at different time points after AAV transduction. For relative gene expression calculation, the gene expression of PBS group in each timepoint was normalized to 1, which was not shown in graph.

FIGS. 6A and 6B show the dsRNA innate immune response in human primary hepatocytes after dsAAV2/hFIX-opt transduction. Fresh human primary hepatocytes from 10 individuals were transduced by dsAAV8/hFIX-opt with 5×10³ particles per cell. The expression of MDA5, RIG-I and IFN-β was detected by Q-PCR at different time points after AAV transduction. For relative gene expression calculation, the gene expression of PBS group in each timepoint was normalized to 1, which was not shown in graph.

FIGS. 7A-7C show the dsRNA response in human hepatocytes from xenografted mice after dsAAV8/hFIX-opt transduction. (7A) 2 human hepatocytes from xenografted mice were injected with 3×10¹¹ particles of AAV8/hFIX-opt. The expression of MDA5, RIG-I and IFN-β of human hepatocytes in mice were detected by Q-PCR at 8 weeks after AAV transduction. MDA5 protein in mice liver was detected by western blot after 8 weeks, the band intensity were measured to show the relative MDA5 expression based on β-actin, in which the data was from 3 separate experiments. **p<0.01, when compared to the control group. (7B) 2 xenograft mice with human hepatocytes from another donor were injected with a dose of dsAAV8/hFIX-opt. The expression of MDA5, RIG-I and IFN-β of human hepatocytes in mice was detected by Q-PCR at 4 and 8 weeks after AAV transduction. (7C) MDA5 protein in mice liver was detected by western blot after 4 or 8 weeks, the relative expression level of MDA5 were calculated based on β-actin intensity, *p<0.05, when compared to the control group.

FIGS. 8A-8E show knockdown of dsRNA activation pathway increased AAV transgene expression. (8A) HeLa cells were transfected with siControl, siMDA5 or siMAVS. The knock down efficiency was detected by western blot and Q-PCR. (8B) At day 0, HeLa cells were transduced with 5×10³ particles of AAV2/luciferase per cell. SiRNA was transfected to HeLa cells at day 4, and luciferase expression was detected 48 h or 72 h later. As control, 2 μg/mL poly(I:C) was added at day 3 and siRNA were transfected to HeLa cells at day 4. *p<0.05, **p<0.01, **p<0.001, when compared to the PBS group. (8C) after 4 days of AAV transduction, siRNA were transfected to HeLa cells, and IFN-β expression was detected by Q-PCR at 48 h post siRNA transfection. **p<0.01, when compared to the PBS group. (8D) after 4 days of AAV transduction, siRNA and IFN-β promoter reporter plasmid were co-transfected to HeLa cells, then luciferase activity were measured after 72 h. (8E) MDA5 expression was detected by Q-PCR at 48 h post siRNA transfection.

FIG. 9 shows the effect of 3′-ITR on transgene expression. 1×10⁵ of 293 cells/well were plated in a 24 well plate. Twenty four hours later, 0.5 up of human alpha-1 antitrypsin (AAT) expression plasmids flanked by two AAV ITRs (2TR) or with 3′ITR deletion (up/TR) or with poly(A) at reversed orientation between transgene and 3′-ITR (2TR/down-poly A-R) were transfected into 293 cells using lipofectamine 2000. At 48 hr post-transfection, AAT level in the supernatant was detected using ELISA. *p<0.05, **p<0.01, when compared to 2TR plasmid.

FIGS. 10A-10B show diagrams of cassettes with (10A) single poly(A) blocking and (10B) multiple poly(A) blocking.

FIG. 11 shows a diagram of ITRs from AAV2 and AAV5.

FIG. 12 shows GFP expression from AAV ITR promoters. 5 ug of pTR/GFP were cotransfected with 1 ug of pCMV/lacZ into 293 cells in a 6 well plate. Two days later, 293 cells were visualized under fluorescence microscopy and stained for LacZ expression.

FIG. 13 shows AAT expression from AAV ITR promoters. 2 ug of pTR/AAT were transfected into different cells in a 12 well plates. Two days later, supernatant was harvested for AAT detection using ELISA.

FIG. 14 shows AAT expression from AAV/ITR-AAT vectors. 1×10e9 particles of AAV/ITR/AAT vectors were added 1×10e5 293 cells in a 48 well plate. Two days later, supernatant was harvested for AAT expression.

FIG. 15 shows 1×10e11 particles of AAV/ITR/AAT vectors were administered via muscular injection in C57BL mice. Four weeks later, the blood was harvested and AAT expression was detected by ELISA.

FIG. 16 shows diagrams A-F of locations for shRNA or miRNA.

FIG. 17 shows diagrams A-C of cassettes for inhibitor expression.

FIG. 18 shows the effect of hydrocortisone on AAV transduction at later time point. Hela cells were transduced with AAV2/luc and 10 ug hydrocortisone was added to culture at day 5 post AAV transduction. 24 hr or 48 hr later after addition of hydrocortisone, luciferase activity from cell lysate was measured.

FIG. 19 shows the effect of hydrocortisone on innate immune response from AAV transduction at later time point. Hela cells were transduced with AAV2/luc and 10 ug hydrocortisone was added to culture at day 5 post AAV transduction. 24 hr later after addition of hydrocortisone, cells were harvested for analysis of MDA5 (top panel) and IFN-β (bottom panel) expression at transcription level by quantitative RT-PCR.

FIGS. 20A-20B show strand transcript generation in AAV-transduced cells. (20A) Overview of the gene-specific reverse transcription to detect either plus or minus strand transcripts. HeLa cells were harvested at day 8 after AAV2/luciferase transduction. The RNA was extracted and treated with DNase. Specific primers for plus strand or minus strand luciferase were used to synthesize different orientations of the cDNA. PCR was performed to detect the transcripts in different orientations of cDNA using primer pair 1 (F1 and R1) and primer pair 2 (F2 and R2). (20B) PCR products are shown. PBS was used as a negative control with no AAV virus. The pTR/luciferase plasmid served as positive control for the PCR. RNA was used as a template to eliminate the possibility of AAV genome DNA contamination in extracted RNA. To measure the yield of transcripts, cDNA in different orientations was diluted to 20-, 200-, or 2,000-fold as PCR templates.

FIG. 21 shows high AAV transduction in human hepatocytes with MAVS deficiency. Human hepatocyte cell lines PH5CH8 and PH5CH8 with MAVS knockdown were transduced with different doses per cell of AAV2/luc vectors. Top panel: 200 vg/cell dose; middle panel: 5000 vg/cell dose; bottom panel: 5000 vg/cell dose. Transgene expression was analyzed at indicated time points.

FIGS. 22A-22B show shRNAs used and a western blot for MAVS shRNA knockdown efficiency. Five different MAVS shRNAs were transfected into Hela cells, and 48 hrs later, cells were collected for cell lysate preparation. Cell lysate was loaded onto a SDS-PAGE gel, and afterward transferred to a nitrocellulose membrane and stained with MAVS antibody and GAPDH antibody. Signal was detected using ECL Western Blotting Detection Reagent (GE). (22A) Sequences of MAVS shRNAs. MAVS shRNA #29: SEQ ID NO:36; MAVS shRNA #30: SEQ ID NO:37; MAVS shRNA #31: SEQ ID NO:38; MAVS shRNA #32: SEQ ID NO:39; MAVS shRNA #68: SEQ ID NO:40. (22B) Western Blot of MAVS and GAPDH.

FIG. 23 shows that knockdown of MAVS with shRNA increases AAV transduction. Hela cells were transfected with MAVS shRNA #31 on day −1, then AAV2/luc vectors at a dose of 5000/cell were added at day 0. At day 1 and 4 post AAV infection, transgene expression was assayed.

### DETAILED DESCRIPTION OF THE INVENTION

The present invention will now be described more fully hereinafter with reference to the accompanying drawings and specification, in which preferred embodiments of the invention are shown. This invention may, however, be embodied in different forms and should not be construed as limited to the embodiments set forth herein.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references and accession numbers cited herein are incorporated by reference in their entireties for the teachings relevant to the sentence and/or paragraph in which the reference is presented.

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a non-viral vector) and the like, is meant to encompass variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461, 463 (CCPA 1976) (emphasis in the original); see also MPEP § 2111.03. Thus, the term “consisting essentially of” when used in a claim of this invention is not intended to be interpreted to be equivalent to “comprising.”

Aspects of the invention relate to the finding that AAV administration induces an innate immune response in a subject resulting from long term AAV transduction. This innate immune response is late in the infection stage. Without being bound by theory, it is believed that the innate immune response is triggered, at least in part, by the presence of double stranded RNA that results from viral infection and/or replication, triggering the cytoplasmic ds RNA recognition pathway. As such, the innate immune response is activated when high amounts of minus stranded RNA are synthesized by the AAV (e.g., at the late phase of AAV transduction). This may be at its peak around week 6 of the transduction. This innate immune response involves, at least in part, increased production of type I IFN-β, and/or increased dsRNA sensors (e.g., MDA5 and MAVS) in the recipient cell or subject. Inhibition of the innate immune response at a late phase following AAV transduction, such as by inhibiting the expression and/or activity of dsRNA sensors, increases AAV transgene expression in the cell or subject.

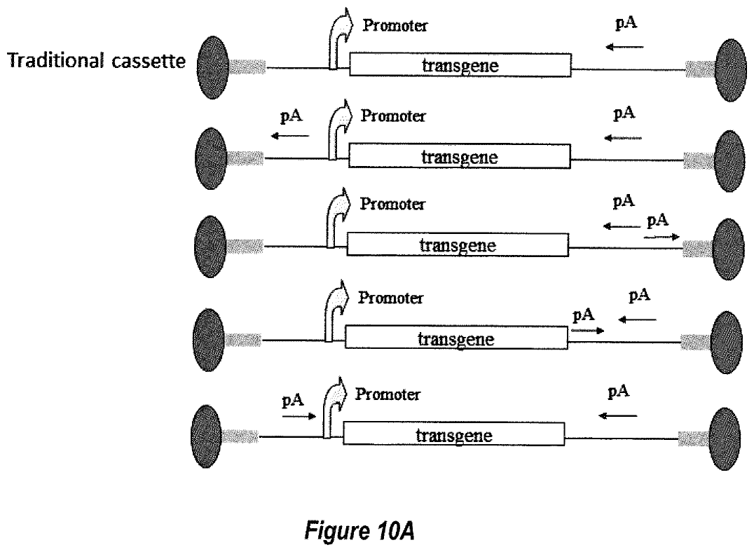

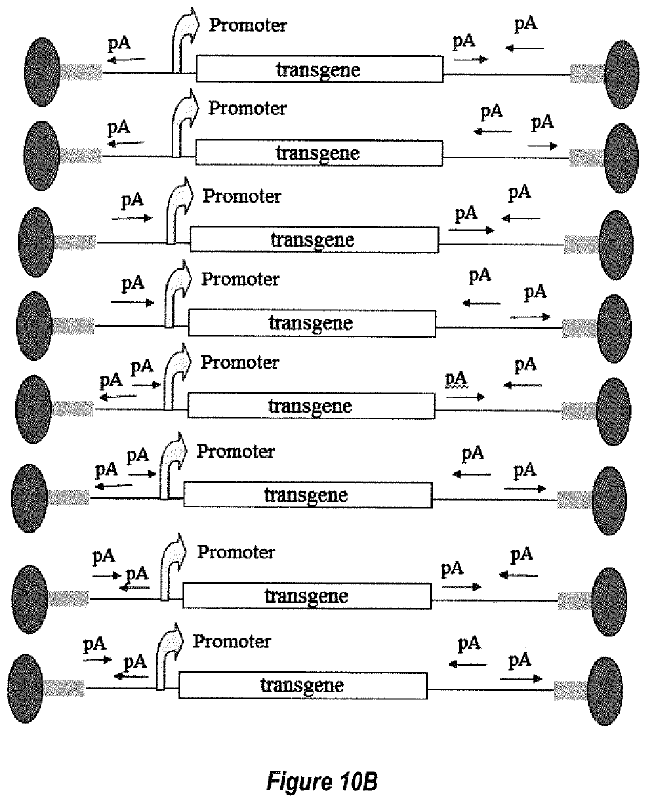

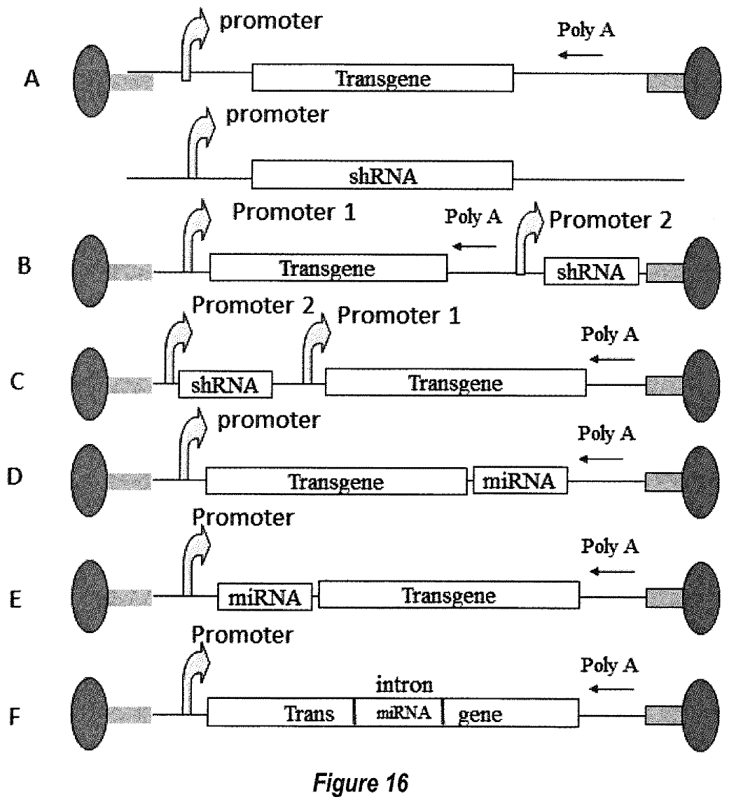

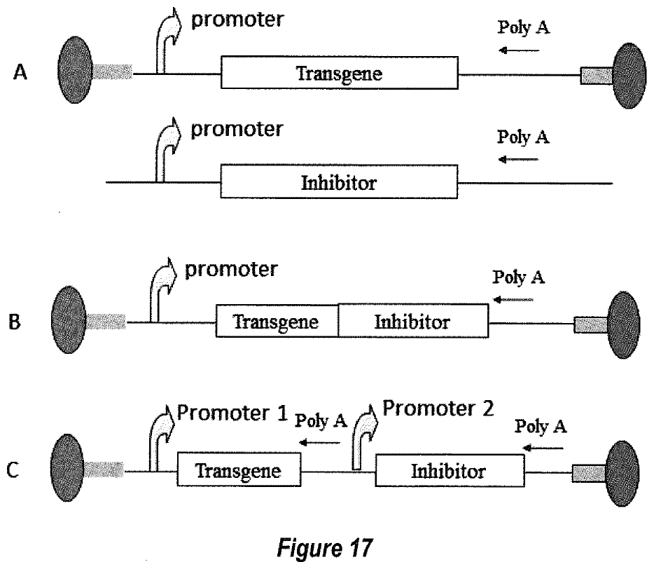

One aspect of the invention relates to a nucleic acid molecule cassette designed to reduce the generation of dsRNA in AAV transduction to thereby reduce provocation of the innate immune response, and/or to inhibit an innate immune response that may be generated (e.g., by expressing RNAi, such as siRNA, that specifically targets mediators of the response, such as MDA5 and/or MAVS). Various forms of these cassettes are described herein (e.g., shown in FIG. 10A and/or FIG. 10B and/or FIG. 16 and/or FIG. 17). Another aspect of the invention relates to an rAAV vector genome that comprises a nucleic acid molecule cassette as described herein (e.g., shown in FIG. 10A and/or FIG. 10B and/or FIG. 16 and/or FIG. 17). The AAV genome that contains the nucleic acid molecule cassette may be further packaged into a viral capsid to form a rAAV particle. Another aspect of the invention relates to a pharmaceutical formulation comprising an rAAV vector genome or AAV particle that comprises a nucleic acid molecule cassette as described herein.

In one embodiment, infection with the rAAV viral particle comprising the nucleic acid molecule cassette results in significant reduction in the innate immune response in the recipient cell or subject, at the late phase of viral transduction, compared to an otherwise identical rAAV viral particle that lacks the cassette elements described herein. In one embodiment, infection with the rAAV viral particle comprising the nucleic acid molecule cassette results in a significant increase in expression of a transgene in a recipient cell or subject, at the late phase of viral transduction, compared to an otherwise identical control rAAV viral particle that lacks the cassette elements described herein. A significant increase is any reproducible, statistically significant increase, such as by the methods used in the examples section herein (e.g., 5%, 10%, 15%, 20%, 25%, 30%, 35%, 40%, 50%, 60%, 75%, 90%, 100%, 2×, 3×, 4×, 5×, 10×, or more increase over the control).

In one embodiment, the present invention provides a recombinant nucleic acid molecule, comprising an adeno-associated virus (AAV) 5′ inverted terminal repeat (ITR), a nucleotide sequence of interest (NOI) operably associated with a promoter and an AAV 3′ ITR, wherein the recombinant nucleic acid molecule further comprises: a) a poly(A) (pA) sequence downstream of the 5′ ITR and upstream of the promoter, in 3′ to 5′ orientation and a pA sequence upstream of the 3′ ITR and downstream of the NOI, in 3′ to 5′ orientation; b) a pA sequence upstream of the 3′ ITR and downstream of the NOI, in 3′ to 5′ orientation; c) a first pA sequence upstream of the 3′ ITR and downstream of the NOI, in 3′ to 5′ orientation and a second pA sequence downstream of the first pA sequence and upstream of the 3′ ITR, in a 5′ to 3′ orientation; d) a first pA sequence upstream of the 3′ ITR and downstream of the NOI, in 3′ to 5′ orientation and a second pA sequence downstream of the NOI and upstream of the first pA, in a 5′ to 3′ orientation; e) a first pA sequence upstream of the 3′ ITR and downstream of the NOI, in 3′ to 5′ orientation and a second pA sequence downstream of the 5′ ITR and upstream of the promoter, in a 5′ to 3′ orientation; f) a first pA sequence downstream of the 5′ ITR and upstream of the promoter, in 3′ to 5′ orientation, a second pA sequence downstream of the NOI and upstream of a third pA sequence, in 5′ to 3′ orientation and the third pA sequence downstream of the second pA sequence and upstream of the 3′ ITR, in 3′ to 5′ orientation; g) a first pA sequence downstream of the 5′ ITR and upstream of the promoter, in 5′ to 3′ orientation, a second pA sequence downstream of the NOI and upstream of a third pA sequence, in 3′ to 5′ orientation and the third pA sequence downstream of the second pA sequence and upstream of the 3′ ITR, in 5′ to 3′ orientation; h) a first pA sequence downstream of the 5′ ITR and upstream of the promoter, in 5′ to 3′ orientation, a second pA sequence downstream of the NOI and upstream of a third pA sequence, in 5′ to 3′ orientation and the third pA sequence downstream of the second pA sequence and upstream of the 3′ ITR, in 3′ to 5′ orientation; i) a first pA sequence downstream of the 5′ ITR and upstream of the promoter, in 5′ to 3′ orientation, a second pA sequence downstream of the NOI and upstream of a third pA sequence, in 3′ to 5′ orientation and the third pA sequence downstream of the second pA sequence and upstream of the 3′ ITR, in 5′ to 3′ orientation; j) a first pA sequence downstream of the 5′ ITR and upstream of a second pA sequence, in 3′ to 5′ orientation, the second pA sequence downstream of the first pA sequence and upstream of the promoter, in 5′ to 3′ orientation; a third pA sequence downstream of the NOI and upstream of a fourth pA sequence, in 5′ to 3′ orientation and the fourth pA sequence downstream of the third pA sequence and upstream of the 3′ ITR, in 3′ to 5′ orientation; k) a first pA sequence downstream of the 5′ ITR and upstream of a second pA sequence, in 3′ to 5′ orientation, the second pA sequence downstream of the first pA sequence and upstream of the promoter, in 5′ to 3′ orientation; a third pA sequence downstream of the NOI and upstream of a fourth pA sequence, in 3′ to 5′ orientation and the fourth pA sequence downstream of the third pA sequence and upstream of the 3′ ITR, in 5′ to 3′ orientation; 1) a first pA sequence downstream of the 5′ ITR and upstream of a second pA sequence, in 5′ to 3′ orientation, the second pA sequence downstream of the first pA sequence and upstream of the promoter, in 3′ to 5′ orientation; a third pA sequence downstream of the NOI and upstream of a fourth pA sequence, in 5′ to 3′ orientation and the fourth pA sequence downstream of the third pA sequence and upstream of the 3′ ITR, in 3′ to 5′ orientation; and/or m) a first pA sequence downstream of the 5′ ITR and upstream of a second pA sequence, in 5′ to 3′ orientation, the second pA sequence downstream of the first pA sequence and upstream of the promoter, in 3′ to 5′ orientation; a third pA sequence downstream of the NOI and upstream of a fourth pA sequence, in 3′ to 5′ orientation and the fourth pA sequence downstream of the third pA sequence and upstream of the 3′ ITR, in 5′ to 3′ orientation.

In one embodiment, the recombinant nucleic acid molecule, comprising an adeno-associated virus (AAV) 5′ inverted terminal repeat (ITR), a nucleotide sequence of interest (NOI) operably associated with a promoter and an AAV 3′ ITR, further comprises: a) a poly A (pA) sequence downstream of the 5′ ITR and upstream of the promoter, in 3′ to 5′ orientation and a pA sequence upstream of the 3′ ITR and downstream of the NOI, in 3′ to 5′ orientation; b) a pA sequence upstream of the 3′ ITR and downstream of the NOI, in 3′ to 5′ orientation; c) a first pA sequence upstream of the 3′ ITR and downstream of the NOT, in 3′ to 5′ orientation and a second pA sequence downstream of the first pA sequence and upstream of the 3′ ITR, in a 5′ to 3′ orientation; d) a first pA sequence upstream of the 3′ ITR and downstream of the NOI, in 3′ to 5′ orientation and a second pA sequence downstream of the NOI and upstream of the first pA, in a 5′ to 3′ orientation; e) a first pA sequence upstream of the 3′ ITR and downstream of the NOI, in 3′ to 5′ orientation and a second pA sequence downstream of the 5′ ITR and upstream of the promoter, in a 5′ to 3′ orientation; f) a first pA sequence downstream of the 5′ ITR and upstream of the promoter, in 3′ to 5′ orientation, a second pA sequence downstream of the NOI and upstream of a third pA sequence, in 5′ to 3′ orientation and the third pA sequence downstream of the second pA sequence and upstream of the 3′ ITR, in 3′ to 5′ orientation; g) a first pA sequence downstream of the 5′ ITR and upstream of the promoter, in 5′ to 3′ orientation, a second pA sequence downstream of the NOI and upstream of a third pA sequence, in 3′ to 5′ orientation and the third pA sequence downstream of the second pA sequence and upstream of the 3′ ITR, in 5′ to 3′ orientation; h) a first pA sequence downstream of the 5′ ITR and upstream of the promoter, in 5′ to 3′ orientation, a second pA sequence downstream of the NOI and upstream of a third pA sequence, in 5′ to 3′ orientation and the third pA sequence downstream of the second pA sequence and upstream of the 3′ ITR, in 3′ to 5′ orientation; i) a first pA sequence downstream of the 5′ ITR and upstream of the promoter, in 5′ to 3′ orientation, a second pA sequence downstream of the NOI and upstream of a third pA sequence, in 3′ to 5′ orientation and the third pA sequence downstream of the second pA sequence and upstream of the 3′ ITR, in 5′ to 3′ orientation; j) a first pA sequence downstream of the 5′ ITR and upstream of a second pA sequence, in 3′ to 5′ orientation, the second pA sequence downstream of the first pA sequence and upstream of the promoter, in 5′ to 3′ orientation; a third pA sequence downstream of the NOT and upstream of a fourth pA sequence, in 5′ to 3′ orientation and the fourth pA sequence downstream of the third pA sequence and upstream of the 3′ ITR, in 3′ to 5′ orientation; k) a first pA sequence downstream of the 5′ ITR and upstream of a second pA sequence, in 3′ to 5′ orientation, the second pA sequence downstream of the first pA sequence and upstream of the promoter, in 5′ to 3′ orientation; a third pA sequence downstream of the NOI and upstream of a fourth pA sequence, in 3′ to 5′ orientation and the fourth pA sequence downstream of the third pA sequence and upstream of the 3′ ITR, in 5′ to 3′ orientation; 1) a first pA sequence downstream of the 5′ ITR and upstream of a second pA sequence, in 5′ to 3′ orientation, the second pA sequence downstream of the first pA sequence and upstream of the promoter, in 3′ to 5′ orientation; a third pA sequence downstream of the NOI and upstream of a fourth pA sequence, in 5′ to 3′ orientation and the fourth pA sequence downstream of the third pA sequence and upstream of the 3′ ITR, in 3′ to 5′ orientation; or m) a first pA sequence downstream of the 5′ ITR and upstream of a second pA sequence, in 5′ to 3′ orientation, the second pA sequence downstream of the first pA sequence and upstream of the promoter, in 3′ to 5′ orientation; a third pA sequence downstream of the NOI and upstream of a fourth pA sequence, in 3′ to 5′ orientation and the fourth pA sequence downstream of the third pA sequence and upstream of the 3′ ITR, in 5′ to 3′ orientation.

In an alternative embodiment, the present invention provides a recombinant nucleic acid molecule, comprising an adeno-associated virus (AAV) 5′ inverted terminal repeat (ITR), a nucleotide sequence of interest (NOI) operably associated with a promoter and an AAV 3′ ITR, wherein the recombinant nucleic acid molecule further comprises one or more of: a) a poly A (pA) sequence downstream of the 5′ ITR and upstream of the promoter, in 3′ to 5′ orientation and a pA sequence upstream of the 3′ ITR and downstream of the NOI, in 3′ to 5′ orientation; b) a pA sequence upstream of the 3′ ITR and downstream of the NOI, in 3′ to 5′ orientation; c) a first pA sequence upstream of the 3′ ITR and downstream of the NOI, in 3′ to 5′ orientation and a second pA sequence downstream of the first pA sequence and upstream of the 3′ ITR, in a 5′ to 3′ orientation; d) a first pA sequence upstream of the 3′ ITR and downstream of the NOI, in 3′ to 5′ orientation and a second pA sequence downstream of the NOI and upstream of the first pA, in a 5′ to 3′ orientation; e) a first pA sequence upstream of the 3′ ITR and downstream of the NOI, in 3′ to 5′ orientation and a second pA sequence downstream of the 5′ ITR and upstream of the promoter, in a 5′ to 3′ orientation; f) a first pA sequence downstream of the 5′ ITR and upstream of the promoter, in 3′ to 5′ orientation, a second pA sequence downstream of the NOI and upstream of a third pA sequence, in 5′ to 3′ orientation and the third pA sequence downstream of the second pA sequence and upstream of the 3′ ITR, in 3′ to 5′ orientation; g) a first pA sequence downstream of the 5′ ITR and upstream of the promoter, in 5′ to 3′ orientation, a second pA sequence downstream of the NOI and upstream of a third pA sequence, in 3′ to 5′ orientation and the third pA sequence downstream of the second pA sequence and upstream of the 3′ ITR, in 5′ to 3′ orientation; h) a first pA sequence downstream of the 5′ ITR and upstream of the promoter, in 5′ to 3′ orientation, a second pA sequence downstream of the NOI and upstream of a third pA sequence, in 5′ to 3′ orientation and the third pA sequence downstream of the second pA sequence and upstream of the 3′ ITR, in 3′ to 5′ orientation; i) a first pA sequence downstream of the 5′ ITR and upstream of the promoter, in 5′ to 3′ orientation, a second pA sequence downstream of the NOI and upstream of a third pA sequence, in 3′ to 5′ orientation and the third pA sequence downstream of the second pA sequence and upstream of the 3′ ITR, in 5′ to 3′ orientation; j) a first pA sequence downstream of the 5′ ITR and upstream of a second pA sequence, in 3′ to 5′ orientation, the second pA sequence downstream of the first pA sequence and upstream of the promoter, in 5′ to 3′ orientation; a third pA sequence downstream of the NOI and upstream of a fourth pA sequence, in 5′ to 3′ orientation and the fourth pA sequence downstream of the third pA sequence and upstream of the 3′ ITR, in 3′ to 5′ orientation; k) a first pA sequence downstream of the 5′ ITR and upstream of a second pA sequence, in 3′ to 5′ orientation, the second pA sequence downstream of the first pA sequence and upstream of the promoter, in 5′ to 3′ orientation; a third pA sequence downstream of the NOI and upstream of a fourth pA sequence, in 3′ to 5′ orientation and the fourth pA sequence downstream of the third pA sequence and upstream of the 3′ ITR, in 5′ to 3′ orientation; 1) a first pA sequence downstream of the 5′ ITR and upstream of a second pA sequence, in 5′ to 3′ orientation, the second pA sequence downstream of the first pA sequence and upstream of the promoter, in 3′ to 5′ orientation; a third pA sequence downstream of the NOI and upstream of a fourth pA sequence, in 5′ to 3′ orientation and the fourth pA sequence downstream of the third pA sequence and upstream of the 3′ ITR, in 3′ to 5′ orientation; or m) a first pA sequence downstream of the 5′ ITR and upstream of a second pA sequence, in 5′ to 3′ orientation, the second pA sequence downstream of the first pA sequence and upstream of the promoter, in 3′ to 5′ orientation; a third pA sequence downstream of the NOI and upstream of a fourth pA sequence, in 3′ to 5′ orientation and the fourth pA sequence downstream of the third pA sequence and upstream of the 3′ ITR, in 5′ to 3′ orientation.

In an alternative embodiment, the present invention provides a recombinant nucleic acid molecule, comprising an adeno-associated virus (AAV) 5′ inverted terminal repeat (ITR), a nucleotide sequence of interest (NOI) operably associated with a promoter and an AAV 3′ ITR, wherein the recombinant nucleic acid molecule further comprises at least one of: a) a poly A (pA) sequence downstream of the 5′ ITR and upstream of the promoter, in 3′ to 5′ orientation and a pA sequence upstream of the 3′ ITR and downstream of the NOI, in 3′ to 5′ orientation; b) a pA sequence upstream of the 3′ ITR and downstream of the NOI, in 3′ to 5′ orientation; c) a first pA sequence upstream of the 3′ ITR and downstream of the NOI, in 3′ to 5′ orientation and a second pA sequence downstream of the first pA sequence and upstream of the 3′ ITR, in a 5′ to 3′ orientation; d) a first pA sequence upstream of the 3′ ITR and downstream of the NOI, in 3′ to 5′ orientation and a second pA sequence downstream of the NOI and upstream of the first pA, in a 5′ to 3′ orientation; e) a first pA sequence upstream of the 3′ ITR and downstream of the NOI, in 3′ to 5′ orientation and a second pA sequence downstream of the 5′ ITR and upstream of the promoter, in a 5′ to 3′ orientation; f) a first pA sequence downstream of the 5′ ITR and upstream of the promoter, in 3′ to 5′ orientation, a second pA sequence downstream of the NOI and upstream of a third pA sequence, in 5′ to 3′ orientation and the third pA sequence downstream of the second pA sequence and upstream of the 3′ ITR, in 3′ to 5′ orientation; g) a first pA sequence downstream of the 5′ ITR and upstream of the promoter, in 5′ to 3′ orientation, a second pA sequence downstream of the NOT and upstream of a third pA sequence, in 3′ to 5′ orientation and the third pA sequence downstream of the second pA sequence and upstream of the 3′ ITR, in 5′ to 3′ orientation; h) a first pA sequence downstream of the 5′ ITR and upstream of the promoter, in 5′ to 3′ orientation, a second pA sequence downstream of the NOI and upstream of a third pA sequence, in 5′ to 3′ orientation and the third pA sequence downstream of the second pA sequence and upstream of the 3′ ITR, in 3′ to 5′ orientation; i) a first pA sequence downstream of the 5′ ITR and upstream of the promoter, in 5′ to 3′ orientation, a second pA sequence downstream of the NOI and upstream of a third pA sequence, in 3′ to 5′ orientation and the third pA sequence downstream of the second pA sequence and upstream of the 3′ ITR, in 5′ to 3′ orientation; j) a first pA sequence downstream of the 5′ ITR and upstream of a second pA sequence, in 3′ to 5′ orientation, the second pA sequence downstream of the first pA sequence and upstream of the promoter, in 5′ to 3′ orientation; a third pA sequence downstream of the NOI and upstream of a fourth pA sequence, in 5′ to 3′ orientation and the fourth pA sequence downstream of the third pA sequence and upstream of the 3′ ITR, in 3′ to 5′ orientation; k) a first pA sequence downstream of the 5′ ITR and upstream of a second pA sequence, in 3′ to 5′ orientation, the second pA sequence downstream of the first pA sequence and upstream of the promoter, in 5′ to 3′ orientation; a third pA sequence downstream of the NOI and upstream of a fourth pA sequence, in 3′ to 5′ orientation and the fourth pA sequence downstream of the third pA sequence and upstream of the 3′ ITR, in 5′ to 3′ orientation; 1) a first pA sequence downstream of the 5′ ITR and upstream of a second pA sequence, in 5′ to 3′ orientation, the second pA sequence downstream of the first pA sequence and upstream of the promoter, in 3′ to 5′ orientation; a third pA sequence downstream of the NOI and upstream of a fourth pA sequence, in 5′ to 3′ orientation and the fourth pA sequence downstream of the third pA sequence and upstream of the 3′ ITR, in 3′ to 5′ orientation; or m) a first pA sequence downstream of the 5′ ITR and upstream of a second pA sequence, in 5′ to 3′ orientation, the second pA sequence downstream of the first pA sequence and upstream of the promoter, in 3′ to 5′ orientation; a third pA sequence downstream of the NOI and upstream of a fourth pA sequence, in 3′ to 5′ orientation and the fourth pA sequence downstream of the third pA sequence and upstream of the 3′ ITR, in 5′ to 3′ orientation.

Nonlimiting examples of embodiments of this invention include the individual cassettes (i.e., recombinant nucleic acid molecules) as shown in FIGS. 10A, 10B, 16 and 17, as well as any cassette having any combination of elements (e.g., poly(A) sequences) and/or any combination of orientations as shown in the respective cassettes. Poly(A) sequences that can be utilized in the invention are known in the art and can be determined by the skilled practitioner. These cassettes and recombinant nucleic acid molecules can be present in a composition or population singly or in any combination and/or in any ratio. A composition or population of this invention can also comprise, consist essentially of or consist of a single cassette or recombinant nucleic acid molecule of this invention.

In another embodiment, the present invention provides a recombinant nucleic acid molecule, comprising an adeno-associated virus (AAV) vector cassette of a first AAV serotype, comprising an AAV 5′ inverted terminal repeat (ITR), a nucleotide sequence of interest (NOI) operably associated with a promoter and an AAV 3′ ITR, wherein the AAV 5′ ITR and/or an AAV 3′ ITR from a second AAV serotype that is different than the first AAV serotype. For example, the 5′ ITR and/or 3′ ITR of the first AAV serotype can be replaced with a 5′ ITR and/or a 3′ ITR from the second AAV serotype.

In further embodiments of the recombinant nucleic acid molecule of this invention, the ITR of the second AAV serotype has no promoter function or reduced promoter function as compared with the promoter function of the ITR of the first AAV serotype. In such embodiments, the first AAV serotype can be any AAV serotype now known or later identified and the second AAV serotype that is different that the first AAV serotype can be any AAV serotype now known or later identified. In some embodiments, the first AAV serotype is AAV2 and the ITR of the second AAV serotype is AAV5. For example, the recombinant nucleic acid molecule can comprise an AAV vector cassette of AAV2, said cassette of AAV2 comprising a 5′ ITR and/or a 3′ ITR of AAV5.

In a further embodiment, the present invention provides a recombinant nucleic acid molecule, comprising an adeno-associated virus (AAV) 5′ inverted terminal repeat (ITR), a nucleotide sequence of interest (NOI) operably associated with a promoter and an AAV 3′ ITR, wherein the 5′ ITR and/or the 3′ ITR that is modified (e.g., by substitution, insertion and/or deletion) to diminish or eliminate promoter activity from the 5′ ITR and/or the 3′ ITR.

In another embodiment, the present invention provides a recombinant nucleic acid molecule, comprising an AAV 5′ ITR, an NOI operably associated with a promoter, a pA sequence in 3′ to 5′ orientation and an AAV 3′ ITR, wherein the NOI sequence is fused with (e.g., in frame with; upstream and/or downstream of) one or more than one nucleotide sequence that encodes an interfering RNA sequence that targets one or more than one cytoplasmic dsRNA sensor.

In some embodiments, the present invention provides A) a recombinant nucleic acid molecule, comprising an AAV 5′ ITR, an NOI operably associated with a first promoter, a first pA sequence in 3′ to 5′ orientation, a nucleotide sequence that encodes an interfering RNA sequence that targets a cytoplasmic dsRNA sensor, operably associated with a second promoter, a second pA sequence and an AAV 3′ ITR; B) A recombinant nucleic acid molecule, comprising an AAV 5′ ITR, a NOI operably associated with a first promoter, a pA sequence in 3′ to 5′ orientation, a short hairpin RNA (shRNA) sequence that targets a cytoplasmic dsRNA sensor, operably associated with a second promoter, and an AAV 3′ ITR; C) a recombinant nucleic acid molecule, comprising an AAV 5′ ITR, a shRNA that targets a cytoplasmic dsRNA sensor, operably associated with a first promoter, a NOI operably associated with a second promoter, a pA sequence in 3′ to 5′ orientation and an AAV 3′ ITR; D) a recombinant nucleic acid molecule, comprising, in the following order: an AAV 5′ ITR, a NOI and a micro RNA (miRNA) sequence that targets a cytoplasmic dsRNA sensor, both operably associated with a promoter, a pA sequence in 3′ to 5′ orientation, and an AAV 3′ ITR; E) A recombinant nucleic acid molecule, comprising, in the following order; an AAV 5′ ITR, a miRNA that targets a cytoplasmic dsRNA sensor and a NOI, both operably associated with a promoter, a pA sequence in 3′ to 5′ orientation, and an AAV 3′ ITR; and/or E) a recombinant nucleic acid molecule, comprising, in the following order: an AAV 5′ ITR, a NOI comprising a miRNA intron sequence within the NOI, the NOI being operably associated with a promoter, a pA sequence in 3′ to 5′ orientation, and an AAV 3′ ITR.

Further provided herein is a composition comprising a first recombinant nucleic acid molecule comprising an AAV 5′ ITR, a NOI operably associated with a promoter, a pA sequence in 3′ to 5′ orientation, and an AAV 3′ ITR and a second recombinant nucleic acid molecule comprising an interfering RNA sequence that targets a cytoplasmic dsRNA sensor.

Nonlimiting examples of a cytoplasmic dsRNA of this invention include MDA5, MAVS, RIG-1, TRAF6, TRAF5, RIP1, FADD, IRF, TRAF3, NAP1, TBK1, IKK, IκB, TANK and any other molecules involved in MAVS downstream signaling, in any combination and order in a recombinant nucleic acid molecule of this invention.

Nonlimiting examples of an interfering RNA (RNAi) of this invention include small interfering RNA (siRNA), short hairpin RNA (shRNA), microRNA (miRNA), long double stranded RNA (long dsRNA), antisense RNA, ribozymes, etc., as are known in the art, as well as any other interfering RNA or inhibitory RNA now known or later identified.

The present invention further provides a recombinant nucleic acid molecule, comprising an AAV 5′ ITR, an NOI and an inhibitor of MAVS signaling, both operably associated with a promoter, a pA sequence in 3′ to 5′ orientation and an AAV 3′ ITR.

Also provided herein is a recombinant nucleic acid molecule, comprising an AAV 5′ ITR, a NOI operably associated with a first promoter, a first pA sequence in 3′ to 5′ orientation, an inhibitor of MAVS signaling operably associated with a second promoter, a second pA sequence in 3′ to 5′ orientation and an AAV 3′ ITR.

In additional embodiments, the present invention provides a composition comprising a first recombinant nucleic acid molecule comprising an AAV 5′ ITR, a NOI operably associated with a promoter, a pA sequence in 3′ to 5′ orientation, and an AAV 3′ ITR and a second recombinant nucleic acid molecule comprising an inhibitor of MAVS signaling and a pA sequence in 3′ to 5′ orientation.

Nonlimiting examples of an inhibitor of MAVS signaling include a serine protease NS3-4A from hepatitis C virus, proteases from Hepatitis A virus and GB virus B, hepatitis B virus (HBV) X protein, poly(rC)-binding protein 2, the 20S proteasomal subunit PSMA7, and/or mitofusin 2, as well as any other inhibitor of MAVS signaling now known or later identified.

A method is also provided herein, of enhancing transduction of an AAV vector in cells of a subject, comprising administering to the subject an AAV vector and an agent that interferes with dsRNA activation pathways in cells of the subject.

Nonlimiting examples of an agent that interferes with dsRNA activation pathways include 2-aminopurine, a steroid (e.g., hydrocortisone), and any other agent that interferes with dsRNA activation pathways in a cell as now known or later identified.

In some embodiments, the AAV vector and the agent(s) of this invention can be administered to the subject simultaneously and/or subsequently, in any order and in any time interval (e.g., hours, days, weeks, etc.) In one embodiment, the AAV vector is administered first, and the agent is administered following that. In one embodiment, the agent is administered first, and the AAV vector is administered following that. In one embodiment, the agent is administered in one or more interval. In one embodiment, the agent is administered in intervals (e.g., days such as every 1, 2, 3, 4, 5, 6, days, or weeks such as every 1, 2, 3, 4, 5, 6 weeks or more) following administration of the AAV vector.

### Definitions

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed (e.g., by negative proviso). For example, in particular embodiments the amino acid is not A, G or I; is not A; is not G or V; etc. as if each such possible disclaimer is expressly set forth herein.

The designation of all amino acid positions in the AAV capsid proteins in the AAV vectors and recombinant AAV nucleic acid molecules of the invention is with respect to VP1 capsid subunit numbering (native AAV2 VP1 capsid protein: GenBank Accession No. AAC03780 or YP680426). It will be understood by those skilled in the art that modifications as described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3).

As used herein, the terms “reduce,” “reduces,” “reduction,” “diminish.” “inhibit” and similar terms mean a decrease of at least about 5%, 10%, 15%; 20%, 25%, 35%, 50%, 75%, 80%, 85%, 90%, 95%, 97% or more.

As used herein, the terms “enhance,” “enhances,” “enhancement” and similar terms indicate an increase of at least about 25%, 50%, 75%, 100%, 150%, 200%, 300%, 400%, 500% or more.

The term “parvovirus” as used herein encompasses the family Parvoviridae, including autonomously replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, B19 virus, and any other autonomous parvovirus now known or later discovered. Other autonomous parvoviruses are known to those skilled in the art. See, e.g., BERNARD N. FIELDS et al., VIROLOGY, Volume 2, Chapter 69 (4th ed., Lippincott-Raven Publishers).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV; equine AAV, ovine AAV, and any other AAV now known or later discovered. See, e.g., BERNARD N. FIELDS et al., VIROLOGY, volume 2, chapter 69 (4th ed., Lippincott-Raven Publishers). A number of additional AAV serotypes and clades have been identified (see, e.g., Gao et al., (2004) J. Virology 78:6381-6388; Moris et al., (2004) Virology 33-:375-383; and Table 3).

The genomic sequences of various serotypes of AAV and the autonomous parvoviruses, as well as the sequences of the native terminal repeats (TRs), Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, NC_001358, NC_001540, AF513851, AF513852, AY530579; the disclosures of which are incorporated by reference herein for teaching parvovirus and AAV nucleic acid and amino acid sequences. See also, e.g., Srivistava et al. (1983) J. Virology 45:555; Chiorini et al., (1998) J. Virology 71:6823; Chiorini et al., (1999) J. Virology 73:1309; Bantel-Schaal et al., (1999) J. Virology 73:939; Xiao et al., (1999) J. Virology 73:3994; Muramatsu et al., (1996) Virology 221:208; Shade et al., (1986) J. Virol. 58:921; Gao et al., (2002) Proc. Nat. Acad. Sci. USA 99:11854; Moris et al., (2004) Virology 33-:375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 3.

The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99:10405-10), AAV4 (Padron et al., (2005) J. Virol. 79: 5047-58), AAV5 (Walters et al., (2004) J. Virol. 78: 3361-71) and CPV (Xie et al., (1996) J. Mol. Biol. 6:497-520 and Tsao et al., (1991) Science 251: 1456-64).

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest.

Unless indicated otherwise, “efficient transduction” or “efficient tropism,” or similar terms, can be determined by reference to a suitable control (e.g., at least about 50%, 60%, 70%, 80%, 85%, 90%, 95%, 100%, 125%, 150%, 175%, 200%, 250%, 300%, 350%, 400%, 500% or more of the transduction or tropism, respectively, of the control). In particular embodiments, the virus vector efficiently transduces or has efficient tropism for neuronal cells and cardiomyocytes. Suitable controls will depend on a variety of factors including the desired tropism and/or transduction profile.

Similarly, it can be determined if a virus “does not efficiently transduce” or “does not have efficient tropism” for a target tissue, or similar terms, by reference to a suitable control. In particular embodiments, the virus vector does not efficiently transduce (i.e., has does not have efficient tropism) for liver, kidney, gonads and/or germ cells. In particular embodiments, transduction (e.g., undesirable transduction) of tissue(s) (e.g., liver) is 20% or less, 10% or less, 5% or less, 1% or less, 0.1% or less of the level of transduction of the desired target tissue(s) (e.g., skeletal muscle, diaphragm muscle, cardiac muscle and/or cells of the central nervous system).

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotides), but in representative embodiments are either single or double stranded DNA sequences.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide. In representative embodiments an “isolated” nucleotide is enriched by at least about 10-fold, 100-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

As used herein, by “isolate” or “purify” (or grammatical equivalents) a virus vector or virus particle or population of virus particles, it is meant that the virus vector or virus particle or population of virus particles is at least partially separated from at least some of the other components in the starting material. In representative embodiments an “isolated” or “purified” virus vector or virus particle or population of virus particles is enriched by at least about 10-fold, 100-fold, 1000-fold, 10,000-fold or more as compared with the starting material.

A “therapeutic polypeptide” is a polypeptide that can alleviate, reduce, prevent, delay and/or stabilize symptoms that result from an absence or defect in a protein in a cell or subject and/or is a polypeptide that otherwise confers a benefit to a subject, e.g., anti-cancer effects or improvement in transplant survivability or induction of an immune response.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is substantially less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

The terms “nucleotide sequence of interest (NOI),” “heterologous nucleotide sequence” and “heterologous nucleic acid molecule” are used interchangeably herein and refer to a nucleic acid sequence that is not naturally occurring in the virus. Generally, the NOT, heterologous nucleic acid molecule or heterologous nucleotide sequence comprises an open reading frame that encodes a polypeptide and/or nontranslated RNA of interest (e.g., for delivery to a cell and/or subject).

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone.

A “recombinant nucleotide sequence,” “recombinant nucleic acid molecule,” “rAAV vector genome” or “rAAV genome” is an AAV genome (i.e., vDNA) that comprises one or more heterologous nucleic acid sequences.

The term “terminal repeat” or “TR” or “inverted terminal repeat (ITR)” includes any viral terminal repeat or synthetic sequence that forms a hairpin structure and functions as an inverted terminal repeat (i.e., mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV terminal repeat” or “AAV TR” may be from any AAV, including but not limited to serotypes 1, 2, 3, 4, 5, 6, 7, 8, 9, 10, 11 or 12 or any other AAV now known or later discovered (see, e.g., Table 3). An AAV terminal repeat need not have the native terminal repeat sequence (e.g., a native AAV TR sequence may be altered by insertion, deletion, truncation and/or missense mutations), as long as the terminal repeat mediates the desired functions, e.g., replication, virus packaging, integration, and/or provirus rescue, and the like. AAV proteins VP1, VP2 and VP3 are capsid proteins that interact together to form an AAV capsid of an icosahedral symmetry. VP1.5 is an AAV capsid protein described in US Publication No. 2014/0037585.

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention.

Further, the viral capsid or genomic elements can contain other modifications, including insertions, deletions and/or substitutions.

A “chimeric” capsid protein as used herein means an AAV capsid protein that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a large number of chimeric capsid proteins are described in the literature as well as herein that can be included in the capsid of this invention.

As used herein, the term “amino acid” or “amino acid residue” encompasses any naturally occurring amino acid, modified forms thereof, and synthetic amino acids.

Naturally occurring, levorotatory (L-) amino acids are shown in Table 4.

Alternatively, the amino acid can be a modified amino acid residue (nonlimiting examples are shown in Table 6) and/or can be an amino acid that is modified by post-translation modification (e.g., acetylation, amidation, formylation, hydroxylation, methylation, phosphorylation or sulfatation).

Further, the non-naturally occurring amino acid can be an “unnatural” amino acid as described by Wang et al., Annu Rev Biophys Biomol Struct. 35:225-49 (2006). These unnatural amino acids can advantageously be used to chemically link molecules of interest to the AAV capsid protein.

In some embodiments, the AAV vector of this invention can be a synthetic viral vector designed to display a range of desirable phenotypes that are suitable for different in vitro and in vivo applications. Thus, in one embodiment, the present invention provides an AAV particle comprising an adeno-associated virus (AAV) capsid, wherein the capsid comprises capsid protein VP1, wherein said capsid protein VP1 is from one or more than one first AAV serotype and capsid protein VP3, wherein said capsid protein VP3 is from one or more than one second AAV serotype and wherein at least one of said first AAV serotype is different from at least one of said second AAV serotype, in any combination.

In some embodiments, the AAV particle can comprise a capsid that comprises capsid protein VP2, wherein said capsid protein VP2 is from one or more than one third AAV serotype, wherein at least one of said one or more than one third AAV serotype is different from said first AAV serotype and/or said second AAV serotype, in any combination. In some embodiments, the AAV capsid described herein can comprise capsid protein VP1.5. VP1.5 is described in US Patent Publication No. 20140037585 and the amino acid sequence of VP1.5 is provided herein.

In some embodiments, the AAV particle of this invention can comprise a capsid that comprises capsid protein VP1.5, wherein said capsid protein VP1.5 is from one or more than one fourth AAV serotype, wherein at least one of said one or more than one fourth AAV serotype is different from said first AAV serotype and/or said second AAV serotype, in any combination. In some embodiments, the AAV capsid protein described herein can comprise capsid protein VP2.

The present invention also provides an AAV vector of this invention, comprising an AAV capsid wherein the capsid comprises capsid protein VP1, wherein said capsid protein VP1 is from one or more than one first AAV serotype and capsid protein VP2, wherein said capsid protein VP2 is from one or more than one second AAV serotype and wherein at least one of said first AAV serotype is different from at least one of said second AAV serotype, in any combination.

In some embodiments, the AAV vector of this invention can comprise a capsid that comprises capsid protein VP3, wherein said capsid protein VP3 is from one or more than one third AAV serotype, wherein at least one of said one or more than one third AAV serotype is different from said first AAV serotype and/or said second AAV serotype, in any combination. In some embodiments, the AAV capsid described herein can comprise capsid protein VP 1.5.

The present invention further provides an AAV vector that comprises an adeno-associated virus (AAV) capsid, wherein the capsid comprises capsid protein VP1, wherein said capsid protein VP1 is from one or more than one first AAV serotype and capsid protein VP1.5, wherein said capsid protein VP1.5 is from one or more than one second AAV serotype and wherein at least one of said first AAV serotype is different from at least one of said second AAV serotype, in any combination.

In some embodiments, the AAV vector of this invention can comprise a capsid that comprises capsid protein VP3, wherein said capsid protein VP3 is from one or more than one third AAV serotype, wherein at least one of said one or more than one third AAV serotype is different from said first AAV serotype and/or said second AAV serotype, in any combination. In some embodiments, the AAV capsid protein described herein can comprise capsid protein VP2.

In some embodiments of the capsid of the AAV vector described herein, said one or more than one first AAV serotype, said one or more than one second AAV serotype, said one or more than one third AAV serotype and said one or more than one fourth AAV serotype are selected from the group consisting of the AAV serotypes listed in Table 3, in any combination.

In some embodiments of the AAV vector of this invention, the AAV capsid described herein lacks capsid protein VP2.

In some embodiments of the AAV vector of this invention, the capsid can comprise a chimeric capsid VP1 protein, a chimeric capsid VP2 protein, a chimeric capsid VP3 protein and/or a chimeric capsid VP1.5 protein.

The present invention further provides a composition, which can be a pharmaceutical formulation comprising the virus vector or AAV particle of this invention and a pharmaceutically acceptable carrier.

In embodiments of the invention, transduction by the AAV particles of this invention of cells is at least about five-fold, ten-fold, 50-fold, 100-fold, 1000-fold or higher than transduction levels by AAV particles that induce a dsRNA mediated immune response as described herein.

Heterologous molecules (e.g., nucleic acid, proteins, peptides, etc.) are defined as those that are not naturally found in an AAV infection, e.g., those not encoded by a wild-type AAV genome. Further, therapeutically useful molecules can be associated with a transgene for transfer of the molecules into host target cells. Such associated molecules can include DNA and/or RNA.

The modified capsid proteins and capsids can further comprise any other modification, now known or later identified. Those skilled in the art will appreciate that for some AAV capsid proteins the corresponding modification will be an insertion and/or a substitution, depending on whether the corresponding amino acid positions are partially or completely present in the virus or, alternatively, are completely absent. Likewise, when modifying AAV other than AAV2, the specific amino acid position(s) may be different than the position in AAV2 (see, e.g., Table 5). As discussed elsewhere herein, the corresponding amino acid position(s) will be readily apparent to those skilled in the art using well-known techniques. Nonlimiting examples of corresponding positions in a number of other AAV are shown in Table 5 (Position 2).

In representative embodiments, the virus vector of this invention is a recombinant virus vector comprising a heterologous nucleic acid encoding a polypeptide and/or a functional RNA of interest. Recombinant virus vectors are described in more detail below.

It will be understood by those skilled in the art that, in certain embodiments, the capsid proteins, virus capsids, virus vectors and AAV particles of the invention exclude those capsid proteins, capsids, virus vectors and AAV particles as they would be present or found in their native state.

### Methods of Producing Virus Vectors

The present invention further provides methods of producing the AAV particles and vectors of this invention. Thus, the present invention provides a method of making an AAV particle, comprising: a) transfecting a host cell with one or more plasmids that provide, in combination all functions and genes needed to assemble AAV particles; b) introducing one or more nucleic acid constructs into a packaging cell line or producer cell line to provide, in combination, all functions and genes needed to assemble AAV particles; c) introducing into a host cell one or more recombinant baculovirus vectors that provide in combination all functions and genes needed to assemble AAV particles; and/or d) introducing into a host cell one or more recombinant herpesvirus vectors that provide in combination all functions and genes needed to assemble AAV particles. Nonlimiting examples of various methods of making the virus vectors of this invention are described in Clement and Greiger (“Manufacturing of recombinant adeno-associated viral vectors for clinical trials” Mol. Ther. Methods Clin Dev. 3:16002 (2016)) and in Greiger et al. (“Production of recombinant adeno-associated virus vectors using suspension HEK293 cells and continuous harvest of vector from the culture media for GMP FIX and FLT1 clinical vector” Mol Ther 24(2):287-297 (2016)), the entire contents of which are incorporated by reference herein.

In one representative embodiment, the present invention provides a method of producing an AAV particle, the method comprising providing to a cell: (a) a nucleic acid template comprising at least one TR sequence (e.g., AAV TR sequence), and (b) AAV sequences sufficient for replication of the nucleic acid template and encapsidation into AAV capsids (e.g., AAV rep sequences and AAV cap sequences encoding the AAV capsids of the invention). Optionally, the nucleic acid template further comprises at least one heterologous nucleic acid sequence. In particular embodiments, the nucleic acid template comprises two AAV ITR sequences, which are located 5′ and 3′ to the heterologous nucleic acid sequence (if present), although they need not be directly contiguous thereto.

The nucleic acid template and AAV rep and cap sequences are provided under conditions such that virus vector comprising the nucleic acid template packaged within the AAV capsid is produced in the cell. The method can further comprise the step of collecting the virus vector from the cell. The virus vector can be collected from the medium and/or by lysing the cells.

The cell can be a cell that is permissive for AAV viral replication. Any suitable cell known in the art may be employed. In particular embodiments, the cell is a mammalian cell. As another option, the cell can be a trans-complementing packaging cell line that provides functions deleted from a replication-defective helper virus, e.g., 293 cells or other E1a trans-complementing cells.

The AAV replication and capsid sequences may be provided by any method known in the art. Current protocols typically express the AAV rep/cap genes on a single plasmid. The AAV replication and packaging sequences need not be provided together, although it may be convenient to do so. The AAV rep and/or cap sequences may be provided by any viral or non-viral vector. For example, the rep/cap sequences may be provided by a hybrid adenovirus or herpesvirus vector (e.g., inserted into the E1a or E3 regions of a deleted adenovirus vector). Epstein Barr virus (EBV) vectors may also be employed to express the AAV cap and rep genes. One advantage of this method is that EBV vectors are episomal, yet will maintain a high copy number throughout successive cell divisions (i.e., are stably integrated into the cell as extra-chromosomal elements, designated as an “EBV based nuclear episome,” see Margolski, (1992) Curr. Top. Microbiol. Immun. 158:67).

As a further alternative, the rep/cap sequences may be stably incorporated into a cell.

Typically the AAV rep/cap sequences will not be flanked by the TRs, to prevent rescue and/or packaging of these sequences.

The nucleic acid template can be provided to the cell using any method known in the art. For example, the template can be supplied by a non-viral (e.g., plasmid) or viral vector. In particular embodiments, the nucleic acid template is supplied by a herpesvirus or adenovirus vector (e.g., inserted into the E1a or E3 regions of a deleted adenovirus). As another illustration, Palombo et al., J. Virology 72:5025 (1998), describes a baculovirus vector carrying a reporter gene flanked by the AAV TRs. EBV vectors may also be employed to deliver the template, as described above with respect to the rep/cap genes.

In another representative embodiment, the nucleic acid template is provided by a replicating rAAV virus. In still other embodiments, an AAV provirus comprising the nucleic acid template is stably integrated into the chromosome of the cell.

To enhance virus titers, helper virus functions (e.g., adenovirus or herpesvirus) that promote a productive AAV infection can be provided to the cell. Helper virus sequences necessary for AAV replication are known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient AAV production as described by Ferrari et al., (1997) Nature Med. 3:1295, and U.S. Pat. Nos. 6,040,183 and 6,093,570.

Further, the helper virus functions may be provided by a packaging cell with the helper sequences embedded in the chromosome or maintained as a stable extrachromosomal element. Generally, the helper virus sequences cannot be packaged into AAV virions, e.g., are not flanked by TRs.

Those skilled in the art will appreciate that it may be advantageous to provide the AAV replication and capsid sequences and the helper virus sequences (e.g., adenovirus sequences) on a single helper construct. This helper construct may be a non-viral or viral construct. As one nonlimiting illustration, the helper construct can be a hybrid adenovirus or hybrid herpesvirus comprising the AAV rep/cap genes.

In one embodiment, the AAV rep/cap sequences and the adenovirus helper sequences are supplied by a single adenovirus helper vector. This vector can further comprise the nucleic acid template. The AAV rep/cap sequences and/or the rAAV template can be inserted into a deleted region (e.g., the E1a or E3 regions) of the adenovirus.

In a further embodiment, the AAV rep/cap sequences and the adenovirus helper sequences are supplied by a single adenovirus helper vector. According to this embodiment, the rAAV template can be provided as a plasmid template.

In another illustrative embodiment, the AAV rep/cap sequences and adenovirus helper sequences are provided by a single adenovirus helper vector, and the rAAV template is integrated into the cell as a provirus. Alternatively, the rAAV template is provided by an EBV vector that is maintained within the cell as an extrachromosomal element (e.g., as an EBV based nuclear episome).

In a further exemplary embodiment, the AAV rep/cap sequences and adenovirus helper sequences are provided by a single adenovirus helper. The rAAV template can be provided as a separate replicating viral vector. For example, the rAAV template can be provided by a rAAV particle or a second recombinant adenovirus particle.

According to the foregoing methods, the hybrid adenovirus vector typically comprises the adenovirus 5′ and 3′ cis sequences sufficient for adenovirus replication and packaging (i.e., the adenovirus terminal repeats and PAC sequence). The AAV rep/cap sequences and if present the rAAV template are embedded in the adenovirus backbone and are flanked by the 5′ and 3′ cis sequences, so that these sequences may be packaged into adenovirus capsids. As described above, the adenovirus helper sequences and the AAV rep/cap sequences are generally not flanked by TRs so that these sequences are not packaged into the AAV virions.

Zhang et al., ((2001) Gene Ther. 18:704-12) describe a chimeric helper comprising both adenovirus and the AAV rep and cap genes.

Herpesvirus may also be used as a helper virus in AAV packaging methods. Hybrid herpesviruses encoding the AAV Rep protein(s) may advantageously facilitate scalable AAV vector production schemes. A hybrid herpes simplex virus type I (HSV-1) vector expressing the AAV-2 rep and cap genes has been described (Conway et al., (1999) Gene Therapy 6:986 and WO 00/17377.

As a further alternative, the virus vectors of the invention can be produced in insect cells using baculovirus vectors to deliver the rep/cap genes and rAAV template as described, for example, by Urabe et al., (2002) Human Gene Therapy 13:1935-43.

AAV vector stocks free of contaminating helper virus may be obtained by any method known in the art. For example, AAV and helper virus may be readily differentiated based on size. AAV may also be separated away from helper virus based on affinity for a heparin substrate (Zolotukhin et al. (1999) Gene Therapy 6:973). Deleted replication-defective helper viruses can be used so that any contaminating helper virus is not replication competent. As a further alternative, an adenovirus helper lacking late gene expression may be employed, as only adenovirus early gene expression is required to mediate packaging of AAV virus. Adenovirus mutants defective for late gene expression are known in the art (e.g., ts100K and ts149 adenovirus mutants).

### Recombinant Virus Vectors

The present invention provides a method of administering a nucleic acid molecule to a cell, the method comprising contacting the cell with the virus vector, the AAV particle, the composition and/or the pharmaceutical formulation of this invention.

The present invention further provides a method of delivering a nucleic acid to a subject, the method comprising administering to the subject the virus vector, the AAV particle, the composition and/or the pharmaceutical formulation of this invention.

The subject of this invention can be any animal and in some embodiments, the subject is a mammal and in some embodiments, the subject is a human. In some embodiments, the subject has or is at risk for a disorder that can be treated by immunotherapy and/or gene therapy protocols. Nonlimiting examples of such disorders include a muscular dystrophy including Duchenne or Becker muscular dystrophy, hemophilia A, hemophilia B, multiple sclerosis, diabetes mellitus, Gaucher disease, Fabry disease, Pompe disease, cancer, arthritis, muscle wasting, heart disease including congestive heart failure or peripheral artery disease, intimal hyperplasia, a neurological disorder including epilepsy, Huntington's disease, Parkinson's disease or Alzheimer's disease, an autoimmune disease, cystic fibrosis, thalassemia, Hurler's Syndrome, Sly syndrome, Scheie Syndrome, Hurler-Scheie Syndrome, Hunter's Syndrome, Sanfilippo Syndrome A, B, C, D, Morquio Syndrome, Maroteaux-Lamy Syndrome, Krabbe's disease, phenylketonuria, Batten's disease, spinal cerebral ataxia, LDL receptor deficiency, hyperammonemia, anemia, arthritis, a retinal degenerative disorder including macular degeneration, adenosine deaminase deficiency, a metabolic disorder, and cancer including tumor-forming cancers.

In the methods described herein, the virus vector, the AAV particle and/or the composition or pharmaceutical formulation of this invention can be administered/delivered to a subject of this invention via a systemic route (e.g., intravenously, intraarterially, intraperitoneally, etc.) and/or local direct injection (e.g., intra-muscular injection, direct brain injection, injection into CSF, injection into eye, etc.). In some embodiments, the virus vector and/or composition can be administered to the subject via an intracerebroventrical, intracisternal, intraparenchymal, intracranial and/or intrathecal route.

The virus vectors of the present invention are useful for the delivery of nucleic acid molecules to cells in vitro, ex vivo, and in vivo. In particular, the virus vectors can be advantageously employed to deliver or transfer nucleic acid molecules to animal cells, including mammalian cells.

Any heterologous nucleic acid sequence(s) of interest may be delivered in the virus vectors of the present invention. Nucleic acid molecules of interest include nucleic acid molecules encoding polypeptides, including therapeutic (e.g., for medical or veterinary uses) and/or immunogenic (e.g., for vaccines) polypeptides.

Therapeutic polypeptides include, but are not limited to, cystic fibrosis transmembrane regulator protein (CFTR), dystrophin (including mini- and micro-dystrophins, see, e.g., Vincent et al., (1993) Nature Genetics 5:130; U.S. Patent Publication No. 2003/017131; International publication WO/2008/088895, Wang et al., Proc. Natl. Acad. Sci. USA 97:13714-13719 (2000); and Gregorevic et al., Mol. Ther. 16:657-64 (2008)), myostatin propeptide, follistatin, activin type II soluble receptor, IGF-1, anti-inflammatory polypeptides such as the Ikappa B dominant mutant, sarcospan, utrophin (Tinsley et al., (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL receptor, lipoprotein lipase, ornithine transcarbamylase, β-globin, α-globin, spectrin, α₁-antitrypsin, adenosine deaminase, hypoxanthine guanine phosphoribosyl transferase, β-glucocerebrosidase, sphingomyelinase, lysosomal hexosaminidase A, branched-chain keto acid dehydrogenase, RP65 protein, cytokines (e.g., α-interferon, β-interferon, interferon-γ, interleukin-2, interleukin-4, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor-3 and -4, brain-derived neurotrophic factor, bone morphogenic proteins [including RANKL and VEGF], glial derived growth factor, transforming growth factor-α and -β, and the like), lysosomal acid α-glucosidase, α-galactosidase A, receptors (e.g., the tumor necrosis growth factora soluble receptor), S100A1, parvalbumin, adenylyl cyclase type 6, a molecule that modulates calcium handling (e.g., SERCA_{2A}, Inhibitor 1 of PP1 and fragments thereof [e.g., WO 2006/029319 and WO 2007/100465]), a molecule that effects G-protein coupled receptor kinase type 2 knockdown such as a truncated constitutively active bARKct, anti-inflammatory factors such as IRAP, anti-myostatin proteins, aspartoacylase, monoclonal antibodies (including single chain monoclonal antibodies; an exemplary Mab is the Herceptin® Mab), neuropeptides and fragments thereof (e.g., galanin, Neuropeptide Y (see, U.S. Pat. No. 7,071,172), angiogenesis inhibitors such as Vasohibins and other VEGF inhibitors (e.g., Vasohibin 2 [see, WO JP2006/073052]). Other illustrative heterologous nucleic acid sequences encode suicide gene products (e.g., thymidine kinase, cytosine deaminase, diphtheria toxin, and tumor necrosis factor), proteins conferring resistance to a drug used in cancer therapy, tumor suppressor gene products (e.g., p53, Rb, Wt-1), TRAIL, FAS-ligand, and any other polypeptide that has a therapeutic effect in a subject in need thereof. AAV vectors can also be used to deliver monoclonal antibodies and antibody fragments, for example, an antibody or antibody fragment directed against myostatin (see, e.g., Fang et al., Nature Biotechnology 23:584-590 (2005)).

Heterologous nucleic acid sequences encoding polypeptides include those encoding reporter polypeptides (e.g., an enzyme). Reporter polypeptides are known in the art and include, but are not limited to, Green Fluorescent Protein (GFP), luciferase, β-galactosidase, alkaline phosphatase, luciferase, and chloramphenicol acetyltransferase gene.

Optionally, the heterologous nucleic acid molecule encodes a secreted polypeptide (e.g., a polypeptide that is a secreted polypeptide in its native state or that has been engineered to be secreted, for example, by operable association with a secretory signal sequence as is known in the art).

Alternatively, in particular embodiments of this invention, the heterologous nucleic acid molecule may encode an antisense nucleic acid molecule, a ribozyme (e.g., as described in U.S. Pat. No. 5,877,022), RNAs that effect spliceosome-mediated trans-splicing (see, Puttaraju et al., (1999) Nature Biotech. 17:246; U.S. Pat. Nos. 6,013,487; 6,083,702), interfering RNAs (RNAi) including siRNA, shRNA or miRNA that mediate gene silencing (see, Sharp et al., (2000) Science 287:2431), and other non-translated RNAs, such as “guide” RNAs (Gorman et al., (1998) Proc. Nat. Acad. Sci. USA 95:4929; U.S. Pat. No. 5,869,248 to Yuan et al.), and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see, e.g., Andino et al., J. Gene Med. 10:132-142 (2008) and Li et al., Acta Pharmacol Sin. 26:51-55 (2005)); phospholamban inhibitory or dominant-negative molecules such as phospholamban S16E (e.g., to treat cardiovascular disease, see, e.g., Hoshijima et al. Nat. Med. 8:864-871 (2002)), RNAi to adenosine kinase (e.g., for epilepsy), and RNAi directed against pathogenic organisms and viruses (e.g., hepatitis B and/or C virus, human immunodeficiency virus, CMV, herpes simplex virus, human papilloma virus, etc.).

Further, a nucleic acid sequence that directs alternative splicing can be delivered. To illustrate, an antisense sequence (or other inhibitory sequence) complementary to the 5′ and/or 3′ splice site of dystrophin exon 51 can be delivered in conjunction with a U1 or U7 small nuclear (sn) RNA promoter to induce skipping of this exon. For example, a DNA sequence comprising a U1 or U7 snRNA promoter located 5′ to the antisense/inhibitory sequence(s) can be packaged and delivered in a modified capsid of the invention.

The virus vector may also comprise a heterologous nucleic acid molecule that shares homology with and recombines with a locus on a host cell chromosome. This approach can be utilized, for example, to correct a genetic defect in the host cell.

The present invention also provides virus vectors that express an immunogenic polypeptide, peptide and/or epitope, e.g., for vaccination. The nucleic acid molecule may encode any immunogen of interest known in the art including, but not limited to, immunogens from human immunodeficiency virus (HIV), simian immunodeficiency virus (SIV), influenza virus, HIV or SIV gag proteins, tumor antigens, cancer antigens, bacterial antigens, viral antigens, and the like.

The use of parvoviruses as vaccine vectors is known in the art (see, e.g., Miyamura et al., (1994) Proc. Nat. Acad. Sci USA 91:8507; U.S. Pat. No. 5,916,563 to Young et al., U.S. Pat. No. 5,905,040 to Mazzara et al., U.S. Pat. No. 5,882,652, U.S. Pat. No. 5,863,541 to Samulski et al.). The antigen may be presented in the parvovirus capsid. Alternatively, the immunogen or antigen may be expressed from a heterologous nucleic acid molecule introduced into a recombinant vector genome. Any immunogen or antigen of interest as described herein and/or as is known in the art can be provided by the virus vector of the present invention.

An immunogenic polypeptide can be any polypeptide, peptide, and/or epitope suitable for eliciting an immune response and/or protecting the subject against an infection and/or disease, including, but not limited to, microbial, bacterial, protozoal, parasitic, fungal and/or viral infections and diseases. For example, the immunogenic polypeptide can be an orthomyxovirus immunogen (e.g., an influenza virus immunogen, such as the influenza virus hemagglutinin (HA) surface protein or the influenza virus nucleoprotein, or an equine influenza virus immunogen) or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pol and env genes products). The immunogenic polypeptide can also be an arenavirus immunogen (e.g., Lassa fever virus immunogen, such as the Lassa fever virus nucleocapsid protein and the Lassa fever envelope glycoprotein), a poxvirus immunogen (e.g., a vaccinia virus immunogen, such as the vaccinia L1 or L8 gene products), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP gene products), a bunyavirus immunogen (e.g., RVFV, CCHF, and/or SFS virus immunogens), or a coronavirus immunogen (e.g., an infectious human coronavirus immunogen, such as the human coronavirus envelope glycoprotein, or a porcine transmissible gastroenteritis virus immunogen, or an avian infectious bronchitis virus immunogen). The immunogenic polypeptide can further be a polio immunogen, a herpes immunogen (e.g., CMV, EBV, HSV immunogens) a mumps immunogen, a measles immunogen, a rubella immunogen, a diphtheria toxin or other diphtheria immunogen, a pertussis antigen, a hepatitis (e.g., hepatitis A, hepatitis B, hepatitis C, etc.) immunogen, and/or any other vaccine immunogen now known in the art or later identified as an immunogen.

Alternatively, the immunogenic polypeptide can be any tumor or cancer cell antigen. Optionally, the tumor or cancer antigen is expressed on the surface of the cancer cell. Exemplary cancer and tumor cell antigens are described in S. A. Rosenberg (Immunity 10:281 (1991)). Other illustrative cancer and tumor antigens include, but are not limited to: BRCA1 gene product, BRCA2 gene product, gp100, tyrosinase, GAGE-1/2, BAGE, RAGE, LAGE, NY-ESO-1, CDK-4, β-catenin, MUM-1, Caspase-8, KIAA0205, HPVE, SART-1, PRAME, p15, melanoma tumor antigens (Kawakami et al., (1994) Proc. Natl. Acad. Sci. USA 91:3515; Kawakami et al., (1994) J. Exp. Med., 180:347; Kawakami et al., (1994) Cancer Res. 54:3124), MART-1, gp100 MAGE-1, MAGE-2, MAGE-3, CEA, TRP-1, TRP-2, P-15, tyrosinase (Brichard et al., (1993) J. Exp. Med. 178:489); HER-2/neu gene product (U.S. Pat. No. 4,968,603), CA 125, LK26, FB5 (endosialin), TAG 72, AFP, CA19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (International Patent Publication No. WO 90/05142); telomerases; nuclear matrix proteins; prostatic acid phosphatase; papilloma virus antigens; and/or antigens now known or later discovered to be associated with the following cancers: melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified (see, e.g., Rosenberg, (1996) Ann. Rev. Med. 47:481-91).

As a further alternative, the heterologous nucleic acid molecule can encode any polypeptide, peptide and/or epitope that is desirably produced in a cell in vitro, ex vivo, or in vivo. For example, the virus vectors may be introduced into cultured cells and the expressed gene product isolated therefrom.

It will be understood by those skilled in the art that the heterologous nucleic acid molecule(s) of interest can be operably associated with appropriate control sequences. For example, the heterologous nucleic acid molecule can be operably associated with expression control elements, such as transcription/translation control signals, origins of replication, polyadenylation signals, internal ribosome entry sites (IRES), promoters, and/or enhancers, and the like.

Further, regulated expression of the heterologous nucleic acid molecule(s) of interest can be achieved at the post-transcriptional level, e.g., by regulating selective splicing of different introns by the presence or absence of an oligonucleotide, small molecule and/or other compound that selectively blocks splicing activity at specific sites (e.g., as described in WO 2006/119137).

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the heterologous nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the heterologous nucleic acid sequence(s). Inducible promoters/enhancer elements for gene delivery can be tissue-specific or -preferred promoter/enhancer elements, and include muscle specific or preferred (including cardiac, skeletal and/or smooth muscle specific or preferred), neural tissue specific or preferred (including brain-specific or preferred), eye specific or preferred (including retina-specific and cornea-specific), liver specific or preferred, bone marrow specific or preferred, pancreatic specific or preferred, spleen specific or preferred, and lung specific or preferred promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the heterologous nucleic acid sequence(s) is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

The virus vectors according to the present invention provide a means for delivering heterologous nucleic acid molecules into a broad range of cells, including dividing and non-dividing cells. The virus vectors can be employed to deliver a nucleic acid molecule of interest to a cell in vitro, e.g., to produce a polypeptide in vitro or for ex vivo or in vivo gene therapy. The virus vectors are additionally useful in a method of delivering a nucleic acid to a subject in need thereof, e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect.

The virus vectors can also be used to produce a polypeptide of interest or functional RNA in cultured cells or in a subject (e.g., using the subject as a bioreactor to produce the polypeptide or to observe the effects of the functional RNA on the subject, for example, in connection with screening methods).

In general, the virus vectors of the present invention can be employed to deliver a heterologous nucleic acid molecule encoding a polypeptide or functional RNA to treat and/or prevent any disorder or disease state for which it is beneficial to deliver a therapeutic polypeptide or functional RNA. Illustrative disease states include, but are not limited to: cystic fibrosis (cystic fibrosis transmembrane regulator protein) and other diseases of the lung, hemophilia A (Factor VIII), hemophilia B (Factor IX), thalassemia (ß-globin), anemia (erythropoietin) and other blood disorders, Alzheimer's disease (GDF; neprilysin), multiple sclerosis (ß-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product, mir-26a [e.g., for hepatocellular carcinoma]), diabetes mellitus (insulin), muscular dystrophies including Duchenne (dystrophin, mini-dystrophin, insulin-like growth factor I, a sarcoglycan [e.g., α, β, γ], RNAi against myostatin, myostatin propeptide, follistatin, activin type II soluble receptor, anti-inflammatory polypeptides such as the Ikappa B dominant mutant, sarcospan, utrophin, mini-utrophin, antisense or RNAi against splice junctions in the dystrophin gene to induce exon skipping [see, e.g., WO/2003/095647], antisense against U7 snRNAs to induce exon skipping [see, e.g., WO/2006/021724], and antibodies or antibody fragments against myostatin or myostatin propeptide) and Becker, Gaucher disease (glucocerebrosidase), Hurler's disease (α-L-iduronidase), adenosine deaminase deficiency (adenosine deaminase), glycogen storage diseases (e.g., Fabry disease [α-galactosidase] and Pompe disease [lysosomal acid α-glucosidase]) and other metabolic disorders, congenital emphysema (al-antitrypsin), Lesch-Nyhan Syndrome (hypoxanthine guanine phosphoribosyl transferase), Niemann-Pick disease (sphingomyelinase), Tays Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration and/or vasohibin or other inhibitors of VEGF or other angiogenesis inhibitors to treat/prevent retinal disorders, e.g., in Type I diabetes), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (I-1) and fragments thereof (e.g., I1C), serca2a, zinc finger proteins that regulate the phospholamban gene, Barkct, β2-adrenergic receptor, β2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S 16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as IRAP and TNFα soluble receptor), hepatitis (α-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like. The invention can further be used following organ transplantation to increase the success of the transplant and/or to reduce the negative side effects of organ transplantation or adjunct therapies (e.g., by administering immunosuppressant agents or inhibitory nucleic acids to block cytokine production). As another example, bone morphogenic proteins (including BNP 2, 7, etc., RANKL and/or VEGF) can be administered with a bone allograft, for example, following a break or surgical removal in a cancer patient.

The invention can also be used to produce induced pluripotent stem cells (iPS). For example, a virus vector of the invention can be used to deliver stem cell associated nucleic acid(s) into a non-pluripotent cell, such as adult fibroblasts, skin cells, liver cells, renal cells, adipose cells, cardiac cells, neural cells, epithelial cells, endothelial cells, and the like. Nucleic acids encoding factors associated with stein cells are known in the art. Nonlimiting examples of such factors associated with stem cells and pluripotency include Oct-3/4, the SOX family (e.g., SOX1, SOX2, SOX3 and/or SOX15), the Klf family (e.g., Klf1, Klf2, Klf4 and/or Klf5), the Myc family (e.g., C-myc, L-myc and/or N-myc), NANOG and/or LIN28.

The invention can also be practiced to treat and/or prevent a metabolic disorder such as diabetes (e.g., insulin), hemophilia (e.g., Factor IX or Factor VIII), a lysosomal storage disorder such as a mucopolysaccharidosis disorder (e.g., Sly syndrome [β-glucuronidase], Hurler Syndrome [α-L-iduronidase], Scheie Syndrome [α-L-iduronidase], Hurler-Scheie Syndrome [α-L-iduronidase], Hunter's Syndrome [iduronate sulfatase], Sanfilippo Syndrome A [heparan sulfamidase], B [N-acetylglucosaminidase], C [acetyl-CoA:α-glucosaminide acetyltransferase], D [N-acetylglucosamine 6-sulfatase], Morquio Syndrome A [galactose-6-sulfate sulfatase], B [β-galactosidase], Maroteaux-Lamy Syndrome [N-acetylgalactosamine-4-sulfatase], etc.), Fabry disease (α-galactosidase), Gaucher's disease (glucocerebrosidase), or a glycogen storage disorder (e.g., Pompe disease; lysosomal acid α-glucosidase).

Gene transfer has substantial potential use for understanding and providing therapy for disease states. There are a number of inherited diseases in which defective genes are known and have been cloned. In general, the above disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, virus vectors according to the present invention permit the treatment and/or prevention of genetic diseases.

The virus vectors according to the present invention may also be employed to provide a functional RNA to a cell in vitro or in vivo. Expression of the functional RNA in the cell, for example, can diminish expression of a particular target protein by the cell. Accordingly, functional RNA can be administered to decrease expression of a particular protein in a subject in need thereof. Functional RNA can also be administered to cells in vitro to regulate gene expression and/or cell physiology, e.g., to optimize cell or tissue culture systems or in screening methods.

In addition, virus vectors according to the instant invention find use in diagnostic and screening methods, whereby a nucleic acid of interest is transiently or stably expressed in a cell culture system, or alternatively, a transgenic animal model.

The virus vectors of the present invention can also be used for various non-therapeutic purposes, including but not limited to use in protocols to assess gene targeting, clearance, transcription, translation, etc., as would be apparent to one skilled in the art. The virus vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

As a further aspect, the virus vectors of the present invention may be used to produce an immune response in a subject. According to this embodiment, a virus vector comprising a heterologous nucleic acid sequence encoding an immunogenic polypeptide can be administered to a subject, and an active immune response is mounted by the subject against the immunogenic polypeptide. Immunogenic polypeptides are as described hereinabove. In some embodiments, a protective immune response is elicited.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to an immunogen by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “protective” immune response or “protective” immunity as used herein indicates that the immune response confers some benefit to the subject in that it prevents or reduces the incidence of disease. Alternatively, a protective immune response or protective immunity may be useful in the treatment and/or prevention of disease, in particular cancer or tumors (e.g., by preventing cancer or tumor formation, by causing regression of a cancer or tumor and/or by preventing metastasis and/or by preventing growth of metastatic nodules). The protective effects may be complete or partial, as long as the benefits of the treatment outweigh any disadvantages thereof.

In particular embodiments, the virus vector or cell comprising the heterologous nucleic acid molecule can be administered in an immunogenically effective amount, as described below.

The virus vectors of the present invention can also be administered for cancer immunotherapy by administration of a virus vector expressing one or more cancer cell antigens (or an immunologically similar molecule) or any other immunogen that produces an immune response against a cancer cell. To illustrate, an immune response can be produced against a cancer cell antigen in a subject by administering a virus vector comprising a heterologous nucleic acid encoding the cancer cell antigen, for example to treat a patient with cancer and/or to prevent cancer from developing in the subject. The virus vector may be administered to a subject in vivo or by using ex vivo methods, as described herein. Alternatively, the cancer antigen can be expressed as part of the virus capsid or be otherwise associated with the virus capsid (e.g., as described above).

As another alternative, any other therapeutic nucleic acid (e.g., RNAi) or polypeptide (e.g., cytokine) known in the art can be administered to treat and/or prevent cancer.

As used herein, the term “cancer” encompasses tumor-forming cancers. Likewise, the term “cancerous tissue” encompasses tumors. A “cancer cell antigen” encompasses tumor antigens.

The term “cancer” has its understood meaning in the art, for example, an uncontrolled growth of tissue that has the potential to spread to distant sites of the body (i.e., metastasize). Exemplary cancers include, but are not limited to melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified. In representative embodiments, the invention provides a method of treating and/or preventing tumor-forming cancers.

The term “tumor” is also understood in the art, for example, as an abnormal mass of undifferentiated cells within a multicellular organism. Tumors can be malignant or benign. In representative embodiments, the methods disclosed herein are used to prevent and treat malignant tumors.

By the terms “treating cancer,” “treatment of cancer” and equivalent terms it is intended that the severity of the cancer is reduced or at least partially eliminated and/or the progression of the disease is slowed and/or controlled and/or the disease is stabilized. In particular embodiments, these terms indicate that metastasis of the cancer is prevented or reduced or at least partially eliminated and/or that growth of metastatic nodules is prevented or reduced or at least partially eliminated.

By the terms “prevention of cancer” or “preventing cancer” and equivalent terms it is intended that the methods at least partially eliminate or reduce and/or delay the incidence and/or severity of the onset of cancer. Alternatively stated, the onset of cancer in the subject may be reduced in likelihood or probability and/or delayed.

It is known in the art that immune responses may be enhanced by immunomodulatory cytokines (e.g., α-interferon, β-interferon, γ-interferon, ω-interferon, τ-interferon, interleukin-1α, interleukin-1β, interleukin-2, interleukin-3, interleukin-4, interleukin 5, interleukin-6, interleukin-7, interleukin-8, interleukin-9, interleukin-10, interleukin-11, interleukin 12, interleukin-13, interleukin-14, interleukin-18, B cell Growth factor, CD40 Ligand, tumor necrosis factor-α, tumor necrosis factor-β, monocyte chemoattractant protein-1, granulocyte-macrophage colony stimulating factor, and lymphotoxin). Accordingly, immunomodulatory cytokines (preferably, CTL inductive cytokines) may be administered to a subject in conjunction with the virus vector.

Cytokines may be administered by any method known in the art. Exogenous cytokines may be administered to the subject, or alternatively, a nucleic acid encoding a cytokine may be delivered to the subject using a suitable vector, and the cytokine produced in vivo.

### Subjects, Pharmaceutical Formulations, and Modes of Administration

Virus vectors and AAV particles according to the present invention find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammal” as used herein includes, but is not limited to, humans, non-human primates, bovines, ovines, caprines, equines, felines, canines, lagomorphs, etc. Human subjects include neonates, infants, juveniles, adults and geriatric subjects.

In representative embodiments, the subject is “in need of” the methods of the invention.

In particular embodiments, the present invention provides a pharmaceutical composition comprising a virus vector and/or capsid and/or AAV particle of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form. For administration to a subject or for other pharmaceutical uses, the carrier will be sterile and/or physiologically compatible.

By “pharmaceutically acceptable” it is meant a material that is not toxic or otherwise undesirable, i.e., the material may be administered to a subject without causing any undesirable biological effects.

One aspect of the present invention is a method of transferring a nucleic acid molecule to a cell in vitro. The virus vector may be introduced into the cells at the appropriate multiplicity of infection according to standard transduction methods suitable for the particular target cells. Titers of virus vector to administer can vary, depending upon the target cell type and number, and the particular virus vector, and can be determined by those of skill in the art without undue experimentation. In representative embodiments, at least about 10³ infectious units, optionally at least about 10⁵ infectious units are introduced to the cell.

The cell(s) into which the virus vector is introduced can be of any type, including but not limited to neural cells (including cells of the peripheral and central nervous systems, in particular, brain cells such as neurons and oligodendricytes), lung cells, cells of the eye (including retinal cells, retinal pigment epithelium, and corneal cells), epithelial cells (e.g., gut and respiratory epithelial cells), muscle cells (e.g., skeletal muscle cells, cardiac muscle cells, smooth muscle cells and/or diaphragm muscle cells), dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. In representative embodiments, the cell can be any progenitor cell. As a further possibility, the cell can be a stem cell (e.g., neural stem cell, liver stem cell). As still a further alternative, the cell can be a cancer or tumor cell. Moreover, the cell can be from any species of origin, as indicated above.

The virus vector can be introduced into cells in vitro for the purpose of administering the modified cell to a subject. In particular embodiments, the cells have been removed from a subject, the virus vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the recombinant virus vector can be introduced into cells from a donor subject, into cultured cells, or into cells from any other suitable source, and the cells are administered to a subject in need thereof (i.e., a “recipient” subject).

Suitable cells for ex vivo nucleic acid delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In particular embodiments, the cells transduced with the virus vector are administered to the subject in a treatment effective or prevention effective amount in combination with a pharmaceutical carrier.

In some embodiments, the virus vector is introduced into a cell and the cell can be administered to a subject to elicit an immunogenic response against the delivered polypeptide (e.g., expressed as a transgene or in the capsid). Typically, a quantity of cells expressing an immunogenically effective amount of the polypeptide in combination with a pharmaceutically acceptable carrier is administered. An “immunogenically effective amount” is an amount of the expressed polypeptide that is sufficient to evoke an active immune response against the polypeptide in the subject to which the pharmaceutical formulation is administered. In particular embodiments, the dosage is sufficient to produce a protective immune response (as defined above). The degree of protection conferred need not be complete or permanent, as long as the benefits of administering the immunogenic polypeptide outweigh any disadvantages thereof.

A further aspect of the invention is a method of administering the virus vector and/or virus capsid to subjects. Administration of the virus vectors and/or capsids according to the present invention to a human subject or an animal in need thereof can be by any means known in the art. Optionally, the virus vector and/or capsid is delivered in a treatment effective or prevention effective dose in a pharmaceutically acceptable carrier.

The virus vectors and/or capsids of the invention can further be administered to elicit an immunogenic response (e.g., as a vaccine). Typically, immunogenic compositions of the present invention comprise an immunogenically effective amount of virus vector and/or capsid in combination with a pharmaceutically acceptable carrier. Optionally, the dosage is sufficient to produce a protective immune response (as defined above). The degree of protection conferred need not be complete or permanent, as long as the benefits of administering the immunogenic polypeptide outweigh any disadvantages thereof. Subjects and immunogens are as described above.

Dosages of the virus vector and/or capsid to be administered to a subject depend upon the mode of administration, the disease or condition to be treated and/or prevented, the individual subject's condition, the particular virus vector or capsid, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10³, 10¹⁴, 10¹⁵ transducing units, optionally about 10⁸ to about 10¹³ transducing units.

In particular embodiments, more than one administration (e.g., two, three, four, five, six, seven, eight, nine, 10, etc., or more administrations) may be employed to achieve the desired level of gene expression over a period of various intervals, e.g., hourly, daily, weekly, monthly, yearly, etc.

Exemplary modes of administration include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). Administration can also be to a tumor (e.g., in or near a tumor or a lymph node). The most suitable route in any given case will depend on the nature and severity of the condition being treated and/or prevented and on the nature of the particular vector that is being used.

The virus vector and/or capsid can be delivered by intravenous administration, intra-arterial administration, intraperitoneal administration, limb perfusion, (optionally, isolated limb perfusion of a leg and/or arm; see, e.g. Arruda et al., (2005) Blood 105: 3458-3464), and/or direct intramuscular injection. In particular embodiments, the virus vector and/or capsid is administered to a limb (arm and/or leg) of a subject (e.g., a subject with muscular dystrophy such as DMD) by limb perfusion, optionally isolated limb perfusion (e.g., by intravenous or intra-articular administration). In embodiments of the invention, the virus vectors and/or capsids of the invention can advantageously be administered without employing “hydrodynamic” techniques. Tissue delivery (e.g., to muscle) of prior art vectors is often enhanced by hydrodynamic techniques (e.g., intravenous/intravenous administration in a large volume), which increase pressure in the vasculature and facilitate the ability of the vector to cross the endothelial cell barrier. In particular embodiments, the viral vectors and/or capsids of the invention can be administered in the absence of hydrodynamic techniques such as high volume infusions and/or elevated intravascular pressure (e.g., greater than normal systolic pressure, for example, less than or equal to a 5%, 10%, 15%, 20%, 25% increase in intravascular pressure over normal systolic pressure). Such methods may reduce or avoid the side effects associated with hydrodynamic techniques such as edema, nerve damage and/or compartment syndrome.

The invention can also be practiced to produce antisense RNA, RNAi or other functional RNA (e.g., a ribozyme) for systemic delivery.

Injectables can be prepared in conventional forms, either as liquid solutions or suspensions, solid forms suitable for solution or suspension in liquid prior to injection, or as emulsions. Alternatively, one may administer the virus vector and/or virus capsids of the invention in a local rather than systemic manner, for example, in a depot or sustained-release formulation. Further, the virus vector and/or virus capsid can be delivered adhered to a surgically implantable matrix (e.g., as described in U.S. Patent Publication No. US-2004-0013645-A1).

In particular embodiments, the delivery vectors of the invention may be administered to treat diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulemia) and cancers and tumors (e.g., pituitary tumors) of the CNS.

Disorders of the CNS include ophthalmic disorders involving the retina, posterior tract, and optic nerve (e.g., retinitis pigmentosa, diabetic retinopathy and other retinal degenerative diseases, uveitis, age-related macular degeneration, glaucoma).

Most, if not all, ophthalmic diseases and disorders are associated with one or more of three types of indications: (1) angiogenesis, (2) inflammation, and (3) degeneration. The delivery vectors of the present invention can be employed to deliver anti-angiogenic factors; anti-inflammatory factors; factors that retard cell degeneration, promote cell sparing, or promote cell growth and combinations of the foregoing.

Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly.

Uveitis involves inflammation. One or more anti-inflammatory factors can be administered by intraocular (e.g., vitreous or anterior chamber) administration of a delivery vector of the invention.

Retinitis pigmentosa, by comparison, is characterized by retinal degeneration. In representative embodiments, retinitis pigmentosa can be treated by intraocular (e.g., vitreal administration) of a delivery vector encoding one or more neurotrophic factors.

Age-related macular degeneration involves both angiogenesis and retinal degeneration. This disorder can be treated by administering the inventive deliver vectors encoding one or more neurotrophic factors intraocularly (e.g., vitreous) and/or one or more anti-angiogenic factors intraocularly or periocularly (e.g., in the sub-Tenon's region).

Glaucoma is characterized by increased ocular pressure and loss of retinal ganglion cells. Treatments for glaucoma include administration of one or more neuroprotective agents that protect cells from excitotoxic damage using the inventive delivery vectors. Such agents include N-methyl-D-aspartate (NMDA) antagonists, cytokines, and neurotrophic factors, delivered intraocularly, optionally intravitreally.

In other embodiments, the present invention may be used to treat seizures, e.g., to reduce the onset, incidence or severity of seizures. The efficacy of a therapeutic treatment for seizures can be assessed by behavioral (e.g., shaking, ticks of the eye or mouth) and/or electrographic means (most seizures have signature electrographic abnormalities). Thus, the invention can also be used to treat epilepsy, which is marked by multiple seizures over time.

In one representative embodiment, somatostatin (or an active fragment thereof) is administered to the brain using a delivery vector of the invention to treat a pituitary tumor. According to this embodiment, the delivery vector encoding somatostatin (or an active fragment thereof) is administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins are known in the art.

In particular embodiments, the vector can comprise a secretory signal as described in U.S. Pat. No. 7,071,172.

In representative embodiments of the invention, the virus vector and/or virus capsid is delivered to the CNS (e.g., to the brain or to the eye) after systemic administration. The virus vector and/or capsid may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes. cortex, basal ganglia, hippocampus and portaamygdala), limbic system, neocortex, corpus striatum, cerebrum, and inferior colliculus. The virus vector and/or capsid may also be delivered to different regions of the eye such as the retina, cornea and/or optic nerve after peripheral administration.

The virus vector and/or capsid may be delivered into the cerebrospinal fluid (e.g., by lumbar puncture) for more disperse administration of the delivery vector. The virus vector and/or capsid may further be administered intravascularly to the CNS in situations in which the blood-brain barrier has been perturbed (e.g., brain tumor or cerebral infarct).

The virus vector and/or capsid can be administered to the desired region(s) of the body by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In particular embodiments, the virus vector is administered in a liquid formulation by direct injection (e.g., stereotactic injection) to the desired region or compartment in the CNS. In other embodiments, the virus vector and/or capsid may be provided by topical application to the desired region or by intra-nasal administration of an aerosol formulation. Administration to the eye may be by topical application of liquid droplets. As a further alternative, the virus vector and/or capsid may be administered as a solid, slow-release formulation (see, e.g., U.S. Pat. No. 7,201,898).

In yet additional embodiments, the virus vector can used for retrograde transport to treat and/or prevent diseases and disorders involving motor neurons (e.g., amyotrophic lateral sclerosis (ALS); spinal muscular atrophy (SMA), etc.). For example, the virus vector can be delivered to muscle tissue from which it can migrate into neurons.

The present invention may be as defined in any one of the following numbered paragraphs:

1. A recombinant nucleic acid molecule, comprising an adeno-associated virus (AAV) 5′ inverted terminal repeat (ITR), a nucleotide sequence of interest (NOI) operably associated with a promoter and an AAV 3′ ITR, wherein the recombinant nucleic acid molecule further comprises:

a) a poly A @A) sequence downstream of the 5′ ITR and upstream of the promoter, in 3′ to 5′ orientation and a pA sequence upstream of the 3′ ITR and downstream of the NOT, in 3′ to 5′ orientation;

b) a pA sequence upstream of the 3′ ITR and downstream of the NOI, in 3′ to 5′ orientation;

c) a first pA sequence upstream of the 3′ ITR and downstream of the NOI, in 3′ to 5′ orientation and a second pA sequence downstream of the first pA sequence and upstream of the 3′ ITR, in a 5′ to 3′ orientation;

d) a first pA sequence upstream of the 3′ ITR and downstream of the NOI, in 3′ to 5′ orientation and a second pA sequence downstream of the NOI and upstream of the first pA, in a 5′ to 3′ orientation;

e) a first pA sequence upstream of the 3′ ITR and downstream of the NOI, in 3′ to 5′ orientation and a second pA sequence downstream of the 5′ ITR and upstream of the promoter, in a 5′ to 3′ orientation;

f) a first pA sequence downstream of the 5′ ITR and upstream of the promoter, in 3′ to 5′ orientation, a second pA sequence downstream of the NOI and upstream of a third pA sequence, in 5′ to 3′ orientation and the third pA sequence downstream of the second pA sequence and upstream of the 3′ ITR, in 3′ to 5′ orientation;

g) a first pA sequence downstream of the 5′ ITR and upstream of the promoter, in 5′ to 3′ orientation, a second pA sequence downstream of the NOI and upstream of a third pA sequence, in 3′ to 5′ orientation and the third pA sequence downstream of the second pA sequence and upstream of the 3′ ITR, in 5′ to 3′ orientation;

h) a first pA sequence downstream of the 5′ ITR and upstream of the promoter, in 5′ to 3′ orientation, a second pA sequence downstream of the NOI and upstream of a third pA sequence, in 5′ to 3′ orientation and the third pA sequence downstream of the second pA sequence and upstream of the 3′ ITR, in 3′ to 5′ orientation;

i) a first pA sequence downstream of the 5′ ITR and upstream of the promoter, in 5′ to 3′ orientation, a second pA sequence downstream of the NOI and upstream of a third pA sequence, in 3′ to 5′ orientation and the third pA sequence downstream of the second pA sequence and upstream of the 3′ ITR, in 5′ to 3′ orientation;

j) a first pA sequence downstream of the 5′ ITR and upstream of a second pA sequence, in 3′ to 5′ orientation, the second pA sequence downstream of the first pA sequence and upstream of the promoter, in 5′ to 3′ orientation; a third pA sequence downstream of the NOT and upstream of a fourth pA sequence, in 5′ to 3′ orientation and the fourth pA sequence downstream of the third pA sequence and upstream of the 3′ ITR, in 3′ to 5′ orientation;

k) a first pA sequence downstream of the 5′ ITR and upstream of a second pA sequence, in 3′ to 5′ orientation, the second pA sequence downstream of the first pA sequence and upstream of the promoter, in 5′ to 3′ orientation; a third pA sequence downstream of the NOI and upstream of a fourth pA sequence, in 3′ to 5′ orientation and the fourth pA sequence downstream of the third pA sequence and upstream of the 3′ ITR, in 5′ to 3′ orientation;

l) a first pA sequence downstream of the 5′ ITR and upstream of a second pA sequence, in 5′ to 3′ orientation, the second pA sequence downstream of the first pA sequence and upstream of the promoter, in 3′ to 5′ orientation; a third pA sequence downstream of the NOI and upstream of a fourth pA sequence, in 5′ to 3′ orientation and the fourth pA sequence downstream of the third pA sequence and upstream of the 3′ ITR, in 3′ to 5′ orientation; and/or

m) a first pA sequence downstream of the 5′ ITR and upstream of a second pA sequence, in 5′ to 3′ orientation, the second pA sequence downstream of the first pA sequence and upstream of the promoter, in 3′ to 5′ orientation; a third pA sequence downstream of the NOI and upstream of a fourth pA sequence, in 3′ to 5′ orientation and the fourth pA sequence downstream of the third pA sequence and upstream of the 3′ ITR, in 5′ to 3′ orientation.

2. A recombinant nucleic acid molecule, comprising an adeno-associated virus (AAV) vector cassette of a first AAV serotype comprising an AAV 5′ inverted terminal repeat (ITR), a nucleotide sequence of interest (NOT) operably associated with a promoter and an AAV 3′ ITR, wherein the AAV 5′ ITR and/or the AAV 3′ ITR is from a second AAV serotype that is different than the first AAV serotype.

3. The recombinant nucleic acid molecule of paragraph 2, wherein the first AAV serotype is AAV2 and the second AAV serotype is AAV5.

4. A recombinant nucleic acid molecule, comprising an adeno-associated virus (AAV) 5′ inverted terminal repeat (ITR), a nucleotide sequence of interest (NOI) operably associated with a promoter and an AAV 3′ ITR, wherein the 5′ ITR and/or the 3′ ITR that is modified to diminish or eliminate promoter activity from the 5′ ITR and/or the 3′ ITR.

5. A recombinant nucleic acid molecule, comprising an AAV 5′ ITR, an NOI operably associated with a promoter, a pA sequence in 3′ to 5′ orientation and an AAV 3′ ITR, wherein the NOI sequence is fused with one or more than one nucleotide sequence that encodes an interfering RNA sequence that targets a cytoplasmic dsRNA sensor.

6. A recombinant nucleic acid molecule, comprising an AAV 5′ ITR, an NOI operably associated with a first promoter, a first pA sequence in 3′ to 5′ orientation, a nucleotide sequence that encodes an interfering RNA sequence that targets a cytoplasmic dsRNA sensor, operably associated with a second promoter, a second pA sequence and an AAV 3′ ITR.

7. A recombinant nucleic acid molecule, comprising an AAV 5′ ITR, a NOI operably associated with a first promoter, a pA sequence in 3′ to 5′ orientation, a short hairpin RNA (shRNA) sequence that targets a cytoplasmic dsRNA sensor, operably associated with a second promoter, and an AAV 3′ ITR.

8. A recombinant nucleic acid molecule, comprising an AAV 5′ ITR, a shRNA that targets a cytoplasmic dsRNA sensor, operably associated with a first promoter, a NOI operably associated with a second promoter, a pA sequence in 3′ to 5′ orientation and an AAV 3′ ITR.

9. A recombinant nucleic acid molecule, comprising, in the following order: an AAV 5′ ITR, a NOI and a micro RNA (miRNA) sequence that targets a cytoplasmic dsRNA sensor, both operably associated with a promoter, a pA sequence in 3′ to 5′ orientation, and an AAV 3′ ITR.

10. A recombinant nucleic acid molecule, comprising, in the following order: an AAV 5′ ITR, a miRNA that targets a cytoplasmic dsRNA sensor and a NOI, both operably associated with a promoter, a pA sequence in 3′ to 5′ orientation, and an AAV 3′ ITR.

11. A recombinant nucleic acid molecule, comprising, in the following order: an AAV 5′ ITR, a NOI comprising a miRNA intron sequence within the NOI, the NOI being operably associated with a promoter, a pA sequence in 3′ to 5′ orientation, and an AAV 3′ ITR.

12. A composition comprising a first recombinant nucleic acid molecule comprising an AAV 5′ ITR, a NOI operably associated with a promoter, a pA sequence in 3′ to 5′ orientation, and an AAV 3′ ITR and a second recombinant nucleic acid molecule comprising an interfering RNA sequence that targets a cytoplasmic dsRNA sensor.

13. The composition of paragraph 12, wherein the interfering RNA sequence is shRNA.

14. A recombinant nucleic acid molecule, comprising:

an AAV 5′ ITR;

a NOI and an inhibitor of MAVS signaling, both operably associated with a promoter;

a pA sequence in 3′ to 5′ orientation; and

an AAV 3′ ITR.

15. The recombinant nucleic acid molecule of paragraph 14, wherein the inhibitor of MAVS signaling is selected from the group consisting of: a serine protease NS3-4A from hepatitis C virus, a protease from Hepatitis A virus, a protease from GB virus B, hepatitis B virus (HBV) X protein, poly(rC)-binding protein 2, the 20S proteasomal subunit PSMA7, mitofusin 2, and any combination thereof.

16. A recombinant nucleic acid molecule, comprising:

an AAV 5′ ITR;

a NOI operably associated with a first promoter;

a first pA sequence in 3′ to 5′ orientation;

an inhibitor of MAVS signaling operably associated with a second promoter;

a second pA sequence in 3′ to 5′ orientation; and

an AAV 3′ ITR.

17. A rAAV vector genome comprising the recombinant nucleic acid molecule of any one of paragraphs 1-12 and 13-16.

18. A rAAV particle comprising the rAAV genome of paragraph 17.

19. A composition comprising the rAAV particle of paragraph 18.

20. A composition comprising a first recombinant nucleic acid molecule comprising an AAV 5′ ITR, a NOI operably associated with a promoter, a pA sequence in 3′ to 5′ orientation, and an AAV 3′ ITR and a second recombinant nucleic acid molecule comprising an inhibitor of MAVS signaling and a pA sequence in 3′ to 5′ orientation.

21. A method of enhancing transduction of an AAV vector in cells of a subject, comprising administering to the subject an AAV vector and an agent that interferes with dsRNA activation pathways in cells of the subject.

22. The method of paragraph 21, wherein the agent that interferes with dsRNA activation pathways in cells of the subject is 2-aminopurine.

23. The method of paragraph 21-22, wherein the AAV vector and the agent are administered to the subject simultaneously.

24. The method of paragraph 21-22, wherein the AAV vector and the agent are administered at separate times.

The present subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### EXAMPLES

The following examples provide illustrative embodiments. Certain aspects of the following examples are disclosed in terms of techniques and procedures found or contemplated by the present inventors to work well in the practice of the embodiments. In light of the present disclosure and the general level of skill in the art, those of skill will appreciate that the following examples are intended to be exemplary only and that numerous changes, modifications, and alterations can be employed without departing from the scope of the presently claimed subject matter.

### Example 1

Cells. HeLa cells, 293 cells, Huh7 cells and HepG2 cells were grown in Dulbecco's Modified Eagle's Medium with 10% FBS and 1% penicillin-streptomycin at 37° C. in 5% CO₂. Human primary hepatocytes were purchased from Triangle Research Labs. The information regarding fresh human primary hepatocytes is listed in Table 1. Primary hepatocytes were plated in Williams' E Medium with Hepatocyte Thawing and Plating Supplement Pack (Thermo Fisher Scientific) and maintained in Williams' E Medium with Hepatocyte Maintenance Supplement Pack and HepExtend™ Supplement (Thermo Fisher Scientific).

AAV Virus Production.

AAV virus production was described before using the triple plasmid transfection. Briefly, HEK-293 cells were transfected with an AAV transgene plasmid (single-stranded (ss) pTR-CBA-Luciferase, double-stranded (ds) pTR-CBh-GFP, ss pTR-CMV-GFP, sspTR-CBA-AAT, dspTR-shRNA-scramble and dspTR-TTR-FIX-opt), a Rep and Cap AAV helper plasmid, and an adenovirus helper plasmid pXX6-80. 48 hours post-transfection, cells were harvested. After lysis of HEK-293 cells, AAV virus was purified by cesium chloride (CsCl) gradient density centrifugation. The virus titer was determined by Q-PCR.

Mice.

Human xenografted mice with 70% human hepatocyte repopulation were purchased from Yecuris company. Mice were maintained in a specific pathogen-free facility at the University of North Carolina at Chapel Hill. The University of North Carolina Institutional Animal Care and Use Committee approved all procedures.

In Vitro Transduction.

HeLa, Huh7, 293 or HepG2 cells were transduced by 5×10³ particles of AAV vector per cell. Transduced cells were harvested at different time points. For long-term AAV transduction study, 1×10⁵ HeLa cells were transduced by 5×10³ particles of AAV per cell in 6 well plate. At day 3 post-transduction, cells were split 1:5, then cells were cultured for at most 5 days with the medium changed every day. AAV transduced cells were harvested at indicated time points and cell lysate was used to measure luciferase activity.

Transduction of Human Primary Hepatocytes.

Suspended hepatocytes were plated to a collagen I coated plate, and AAV vectors (AAV2/GFP or AAV2/FIX-opt) were added in a dose of 5×10³ particles per cell. One day later, plating medium was changed to maintenance medium. Primary hepatocytes were cultured for 10 days while the medium was changed every day. Hepatocytes were harvested at different time points for detection of MDA-5, RIG-1 and IFN-β.

Mouse Experiments.

Human hepatocytes from xenografted mice were administered with 3×10¹¹ particles of AAV8/FIX-opt via retro-orbital injection. At weeks 4 or 8 post AAV injection, mice were sacrificed and livers were harvested for RNA extraction and Protein analysis with Western Blot.

Luciferase Assay.

Cells transduced by AAV2/luciferase were treated with passive lysis buffer (Promega) for 20 min. Luciferase activity was measured with Luciferase Assay Reagent (Promega) following the manufacturer's instructions. Luciferase activity was measured with a Wallac 1420 Victor3 plate reader.

Transfection Assay.

For poly(I:C) transfection, cells were transfected with 2 μg poly(I:C) by Lipofectamin 3000 (Thermo Fisher Scientific) in a 12 well plate at different time points: 18 h prior to AAV transduction, at the same time or 3 days later post AAV transduction. For siRNA transfection, at day 3 post-transduction of AAV vector, HeLa cells were split, and 24 h later, cells were transfected with 1 μg siRNA (siMDA5: CUGAAUCUGCUCCUUCACC (SEQ ID NO:1), siMAVS: AUACAACUGACCCUGUGGG (SEQ ID NO:2), siMAVS-2: UAGUUGAUCUCGCGGACGA (SEQ ID NO:3) and CCGUUUGCUGAAGACAAGA (SEQ ID NO:4), siControl: UGUGAUCAAGGACGCUAUG, SEQ ID NO:5). At 48 or 72 hours after transfection, cells were harvested for luciferase assay or RNA extraction.

RNA Isolation and Real-Time PCR.

RNA from cultured cells or mouse liver tissues was isolated using TRIzol Reagent (Invitrogen). Synthesis of first strand cDNA from RNA templates was performed using RevertAid First Strand cDNA Synthesis Kit (Thermo Fisher Scientific). Real-time PCR was performed by LightCycler 480 instrument (Roche). Primers used in real-time PCR are listed in Table 2.

Western Blot.

Cells or tissues were treated with RIPA buffer. 60 μg of proteins per lane were loaded to an SDS-PAGE gel. After proteins were transferred to an NC membrane, the membrane was stained with Rabbit Monoclonal MDA5 antibody (Thermo Fisher Scientific) or β-actin antibody (Thermo Fisher Scientific). Signal was detected using ECL Western Blotting Detection Reagent (GE). Data analysis was performed using ImageJ software.

IFN-β Promoter Reporter Assay.

1×10⁵ HeLa cells were transduced by 5×10³ particles of AAV2/GFP per cell in 6 well plate. Cells were split in 1:5 at day 3 post-transduction. Twenty four h later, cells were co-transfected with IFN-β promoter reporter plasmid and siRNA. Then luciferase activity was measured after 72 h of transfection.

Statistical Analysis.

All statistical calculations were performed using a statistical software (GraphPad Prism 7.0 software). Differences between different groups, which were evaluated by the Student's t test, were considered to be statistically significant when P values were <0.05.

IFN-β Inhibits Transgene Expression from AAV Transduced Cells.

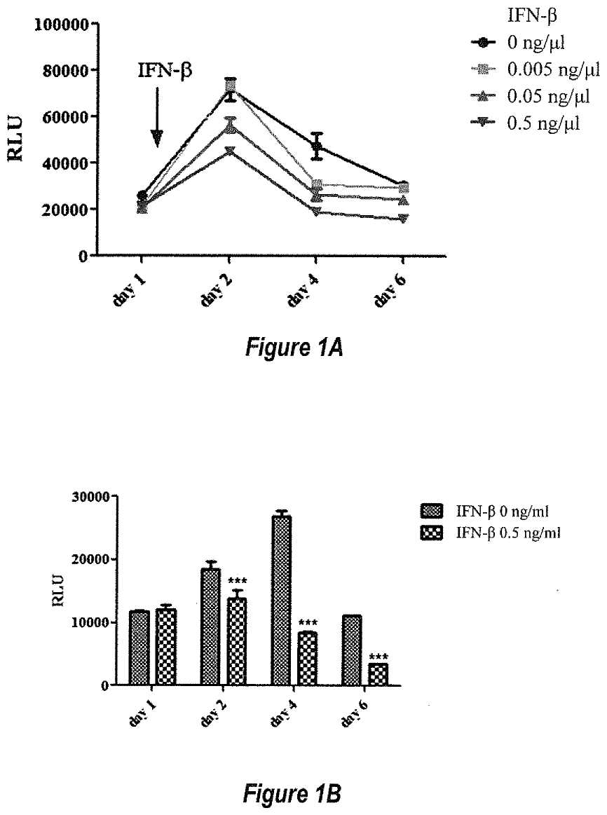

Type I IFN-β expression is the hallmark of innate immune activation. To study the effect of innate immune response activation on transgene expression, we investigated the effect of IFN-β on transgene expression after AAV transduction in vitro. HeLa cells were infected with AAV2/luc vector encoding firefly luciferase transgene, and 24 hr later, IFN-β at different doses was added. At different time points post supplementation of IFN-β, the luciferase activity was measured. Compared to the PBS (no IFN) group, luciferase transgene expression had dramatically decreased. The inhibition was dose-dependent (FIG. 1A). When IFN-β was added every day since dayl post AAV transduction, much stronger inhibition of luciferase expression was observed from a long-term culture (FIG. 1B). This result suggests that innate immune response activation may inhibit AAV transduction.

Poly(I:C) Inhibits AAV Transduction.

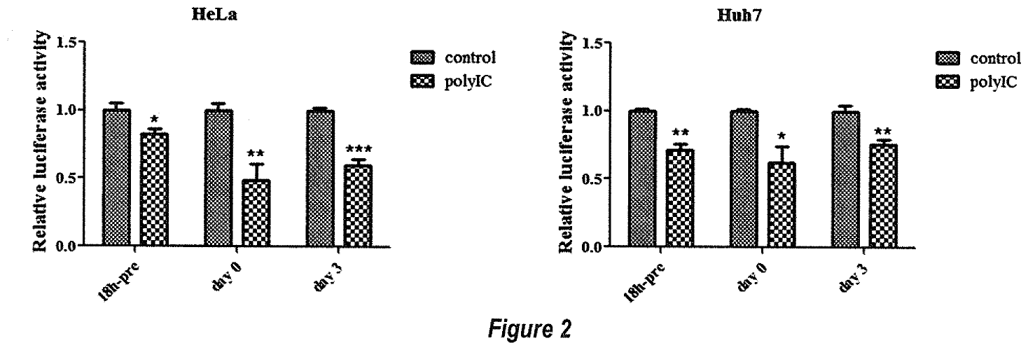

In clinical trials in patients with hemophilia, the decrease of transgene expression was observed at weeks 6 to 10 after AAV administration. At that time point, most AAV virions have already entered the nucleus for effective transgene expression. The possibility of the innate immune response being triggered from the pattern recognition receptors (PRP) in endosomes or sensors for DNA in cytoplasm seems low. We presume that dsRNA could be generated from AAV vector mediated transgene delivery and could activate innate immune response. To determine whether innate immunity from dsRNA impacts transgene expression from AAV transduction, we transduced cells with AAV2/luc with transfection of polyinosinic-polycytidylic acid (poly(I:C)), which is a synthetic analog of dsRNA, at different time points, either at 18 hrs pre-AAV2 transduction or at the same time or at day 3 post-AAV2 transduction. Seventy two hrs later post poly(I:C) transfection, cell lysate was harvested for luciferase activity analysis. Regardless of the time points, transfection of poly(I:C) inhibited transgene expression in both HeLa cells and Huh7 cells (FIG. 2). The data indicates that the innate immune response triggered from dsRNA impacts AAV transduction.

Double-Stranded RNA Innate Immune Response is Triggered from Late AAV transduction in HeLa cells.

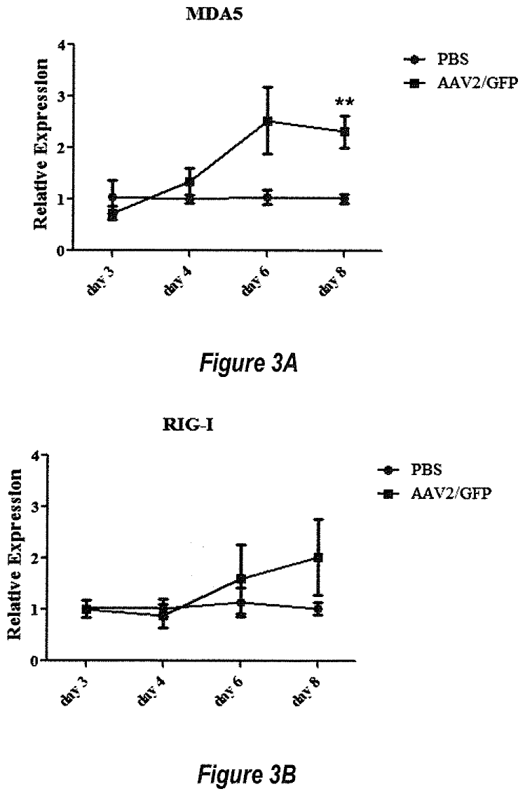

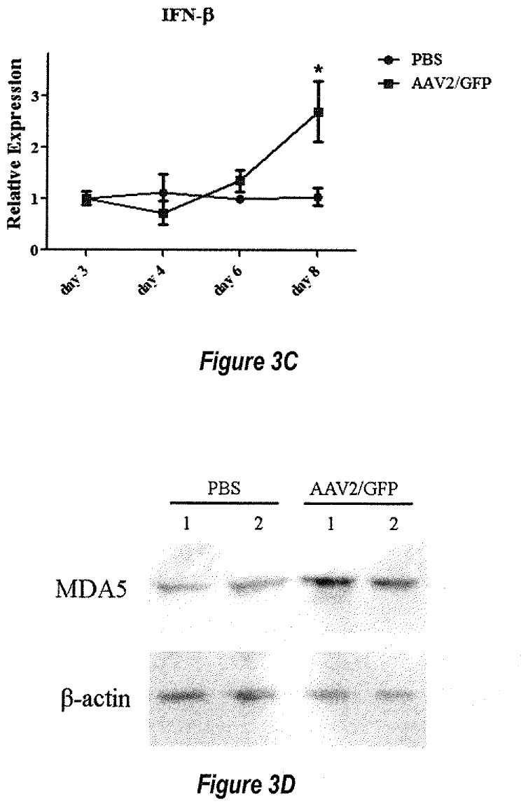

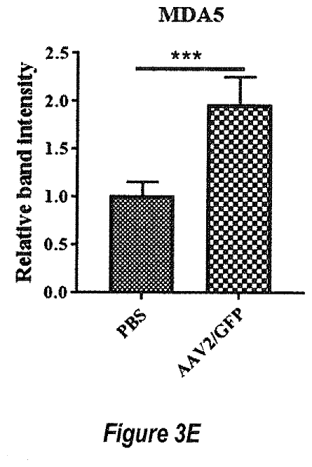

To study whether AAV transduction activates the innate immune response triggered by dsRNA, we examined the expression kinetics of dsRNA sensors at the transcriptional level: MDA5 and RIG1. As shown in FIG. 3, the up-regulation of MDA5 was observed at day 6 post scAAV/GFP vector transduction in HeLa cells. There was no activation of MDA5 before day 5 of transduction (FIG. 3A). RIG1 expression had not increased during AAV transduction in HeLa cells (FIG. 3B). No IFN-β had increased during day 3-6, however high IFN-β expression was obtained at day 8 (FIG. 3C). We also examined the expression of MDA-5 at the translational level. At day 8 post AAV transduction, the MDA-5 expression in the cell lysate was found to be higher based on the intensity of the Western blot results (FIGS. 3D and 3E). This study implicates that AAV transduction is able to activate a dsRNA mediated innate immune response.

AAV Transduction Mediated dsRNA Innate Immune Response Activation is Cell Specific and Transgene Dependent.

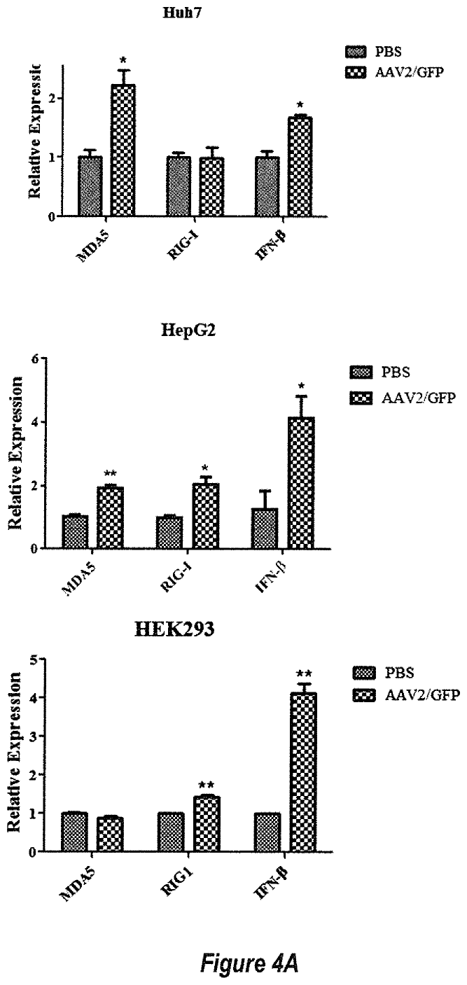

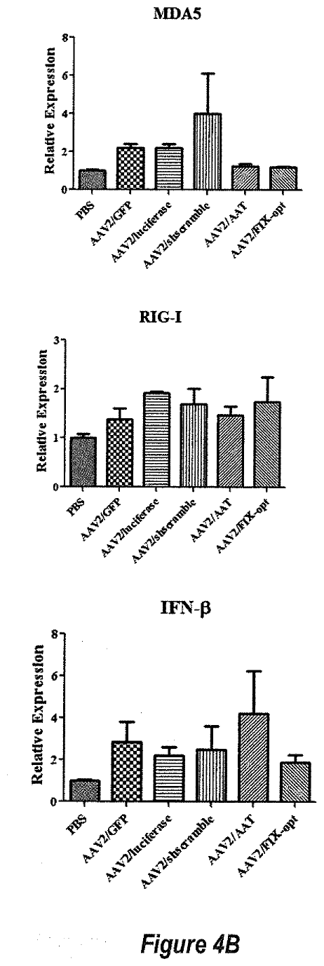

In the clinical trials in patients with hemophilia B, transgene FIX was driven by the liver specific promoter and mainly expressed in the hepatocytes. The results from the studies above have demonstrated that the dsRNA immune response is triggered at a later time after AAV transduction in HeLa cells, a non-hepatocyte cell line. Next, we wonder whether the dsRNA innate immune response was also triggered in other human cell lines, including cell lines that were derived from hepatocytes. After infection of AAV2/GFP vectors, at different time points, dsRNA response was evaluated. Up-regulation of MDA5 and IFN-β was only observed in human hepatocyte Huh7 cells and HepG2 cells, but not in 293 (FIG. 4A).

To study the activation of the dsRNA mediated innate immune response from different transgenes, we transduced HeLa cells with AAV2 encoding different transgenes including luciferase, shRNA-scramble, and antitrypsin. At day 8 post AAV transduction, MDA5 transcription was detected. Compared to the control group, higher expression of MDA-5 was observed in HeLa cells transduced with ssAAV/GFP, AAV2/luc, AAV2/shRNA-scramble, but not with AAV2/AAT (FIG. 4B). However, IFN-β expression was up-regulated in all AAV2 vector transduced cells regardless of transgenes.

The dsRNA Innate Immune Response is Induced in AAV/GFP Transduced Primary Human Hepatocytes.

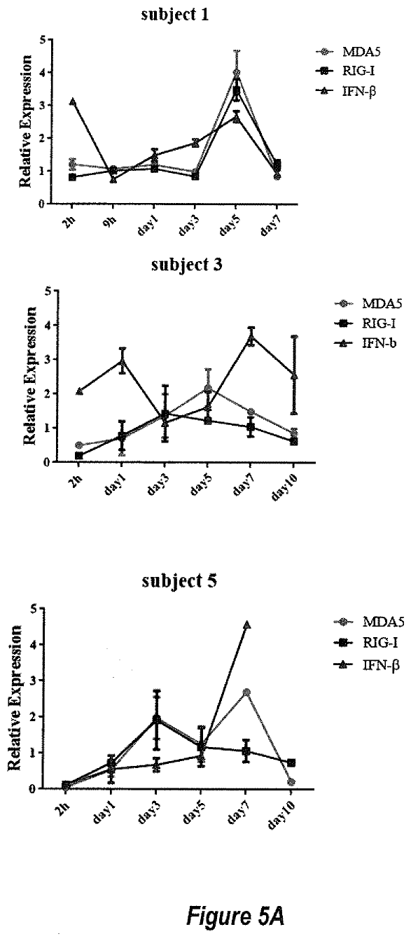

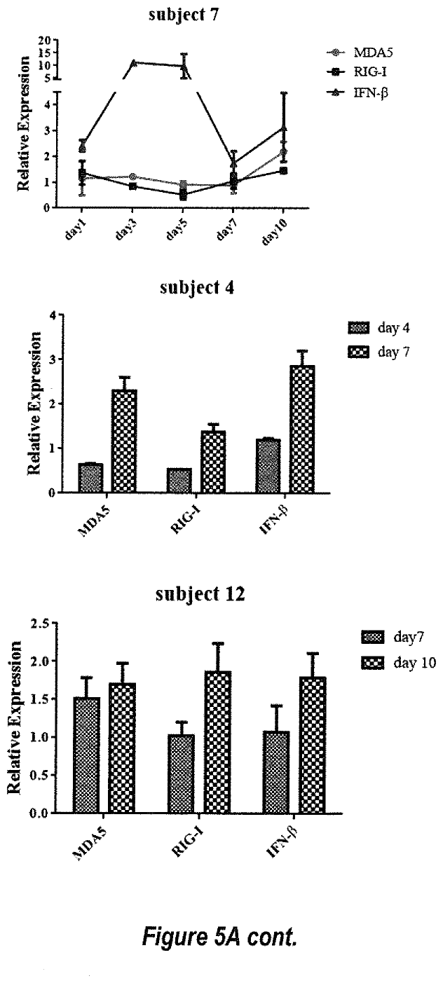

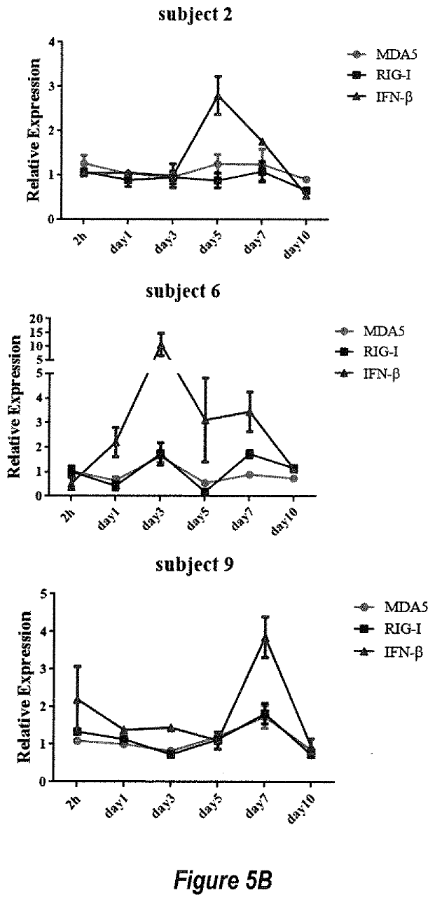

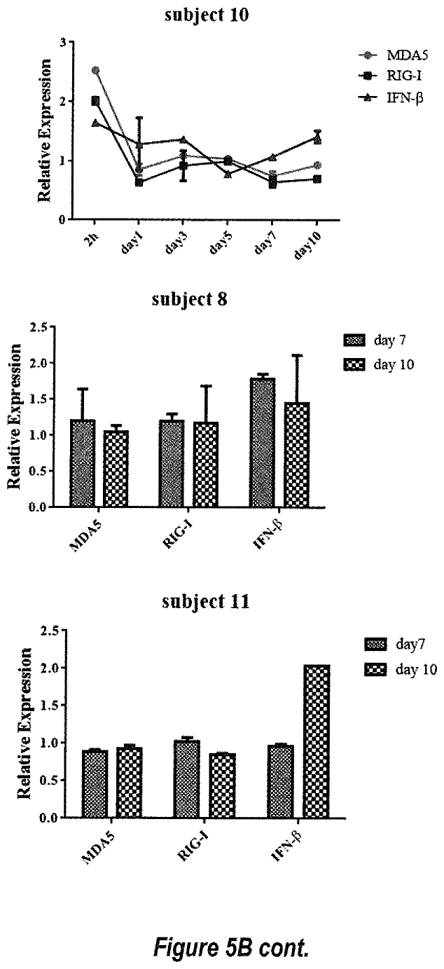

It has been shown that AAV transduction triggered the dsRNA innate immune response in the human hepatocyte cell line, Huh7 cells, and as stated above, we wondered whether the finding was applicable to human primary hepatocytes. It has been demonstrated that AAV2 can efficiently transduce primary human hepatocytes in vitro. We used scAAV2/GFP vectors to transduce primary human hepatocytes from 6 different subjects. At different time points after AAV2 transduction, RNA from human hepatocytes was harvested for transcriptional expression of MDA5, RIG1 and IFN-β. MDA5 was up-regulated in 6 out of 12 subjects beyond day 5 after AAV transduction (FIG. 5A), in which higher expression of RIG1 was only observed in 3 subjects (sub 1, 5 and 12). Another 6 subjects didn't show the expression change of MDA5 or RIG-I (FIG. 5B). However, higher expression of IFN-β was detected in all subjects after AAV transduction (FIG. 5). MDA5 expression reached the peak at day 5 or later after AAV transduction and then decreased to the baseline. There was no specific pattern for high expression of IFN-β, and in most cases, the increased IFN-β expression was accompanied with MDA5 expression at a late time point (>5 days) post AAV transduction. In some subjects, high IFN-β expression was detected in very early time points (within one day) after AAV transductions (sub 1, 3, 7 and 9), but there was no change for dsRNA sensors. This result perhaps supports the innate immune response activation via the dsDNA-TLR9 pathway as reported in other studies.

The dsRNA Innate Immune Response is Induced in AAV/FIX-Opt Transduced Primary Human Hepatocytes.

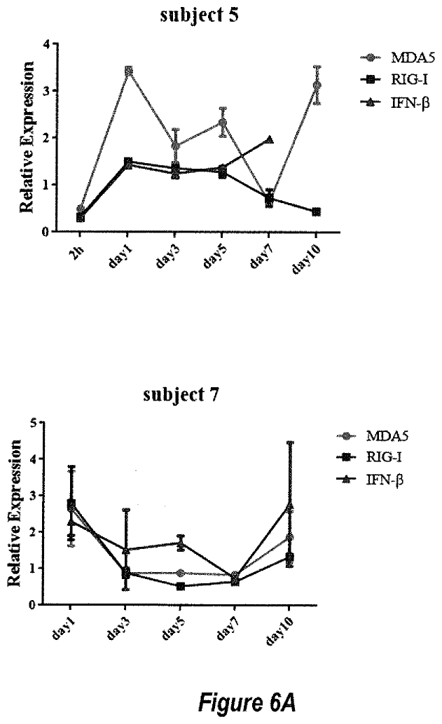

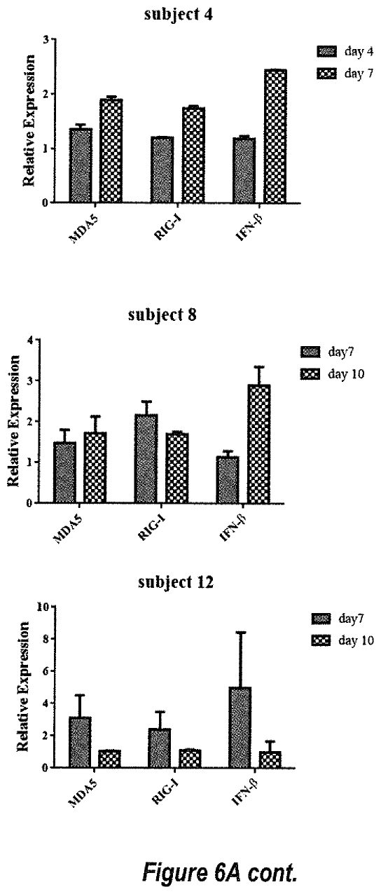

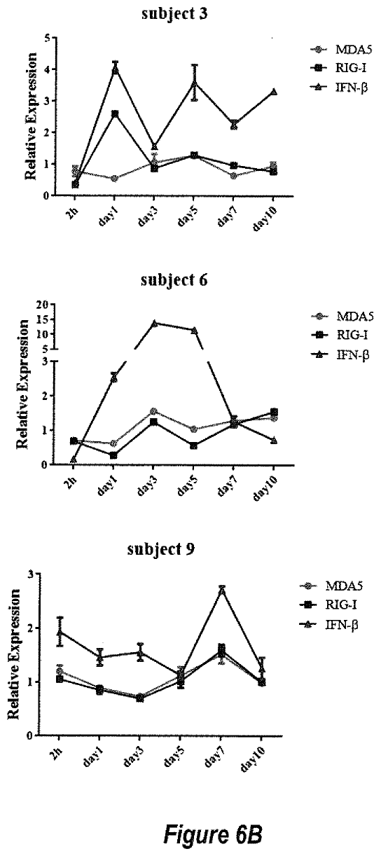

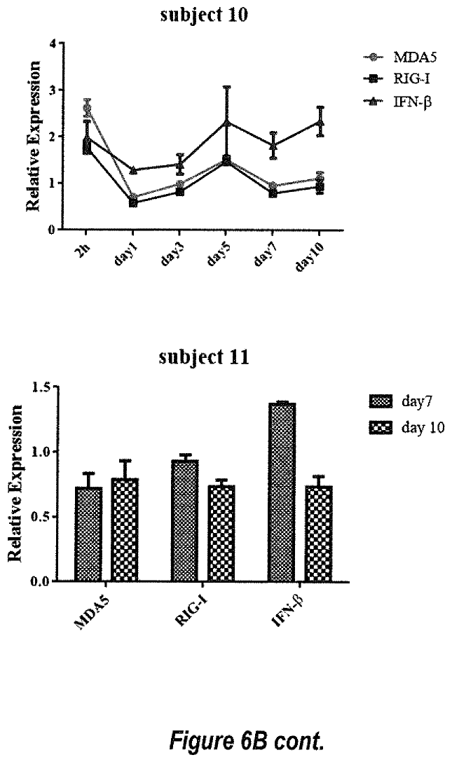

Next, we tested whether AAV vectors to deliver the therapeutic transgene FIX also triggered the dsRNA innate immune response. We transduced human primary hepatocytes from 10 subjects with AAV vector to deliver the clinically used FIX cassette-optimized human FIX with a mutation at R338L for enhanced coagulation activity (hFIX-R338L-opt). After transduction with AAV2/hFIX-R338L-opt, MDA5 and IFN-β was up-regulated in 5 out of 10 subjects at day 5 or beyond after AAV infection (FIG. 6A), and the other 5 subjects just showed only high expression of IFN-β (FIG. 6B). RIG-I was up-regulated in subject 8 and 12 at day 7 post AAV transduction (FIG. 6A). This result indicates that the dsRNA innate immune response is activated in human primary hepatocytes transduced with AAV vectors encoding a clinically therapeutic transgene (FIG. 6).

The Activation of the dsRNA Innate Immune Response in Human Hepatocytes from AAV Transduction in Humanized Mice.

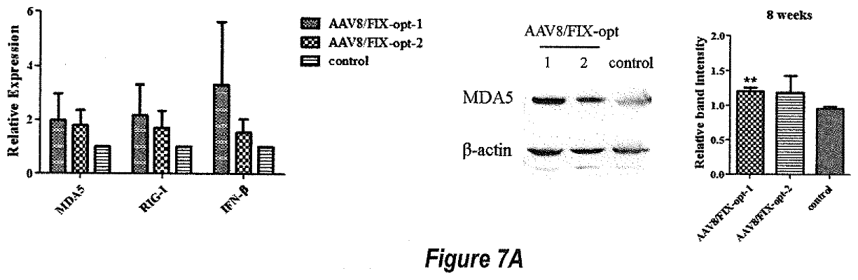

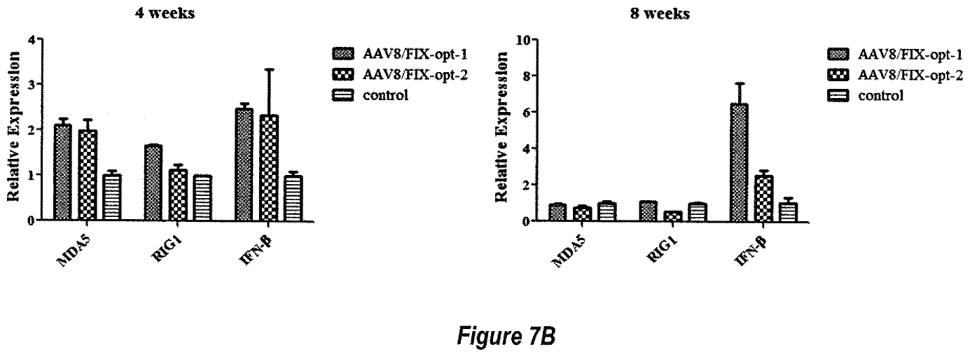

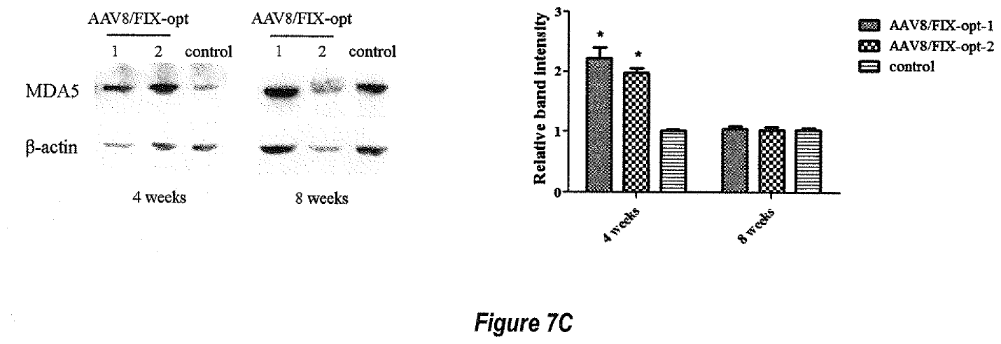

All of the above results support that the cytosolic dsRNA innate immune response in human cells is activated at a late time following AAV transduction in vitro. Next, we examined the dsRNA innate immune response in human hepatocytes in vivo using a human chimeric mouse model. In these mice, human hepatocytes were engrafted into mouse liver with a 70% repopulation. In the first set of experiments, we injected clinical vectors AAV8/hFIX-opt in two mice, and at week 8, mouse liver was harvested for RNA extraction. Then, MDA5 and RIG1 expression at the transcription level were detected in human hepatocytes. As shown in FIG. 7A, the expression of both MDA5 and RIG1 had increased. Also, IFN-β expression was higher in the AAV8/hFIX-opt treated mouse than that in mouse with no treatment. In a second experiment, after AAV8/hFIX-opt injection, at week 4 and 8, mouse livers were harvested for analysis of the dsRNA immune response. The mRNA level for both MDA and RIG-1 had increased at week 4 and decreased to a control level at week 8 after AAV8 administration (FIG. 7B). Higher expression of MDA5 was confirmed at protein level (FIG. 7C). AAV treated mice had high IFN-β expression not only at week 4 but also at week 8 (FIG. 7).

Blockage of the dsRNA Activation Pathway Increases Transgene Expression and Inhibits IFN-β Expression from AAV Transduced Cells. Inhibits IFN-β Expression from AAV Transduced Cells.

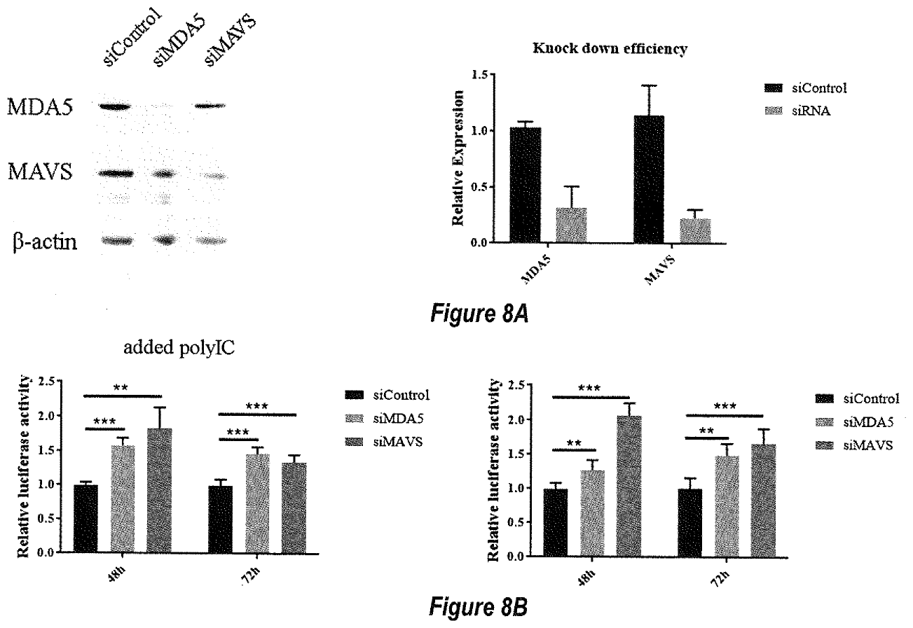

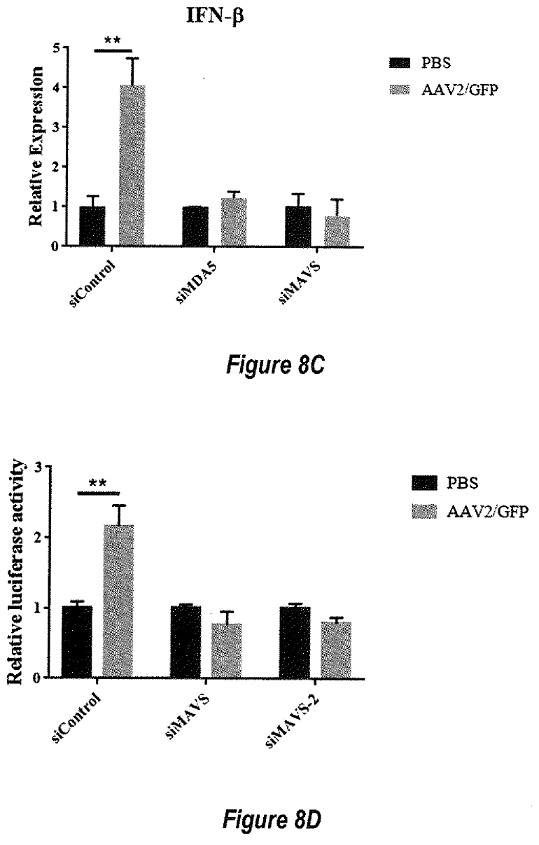

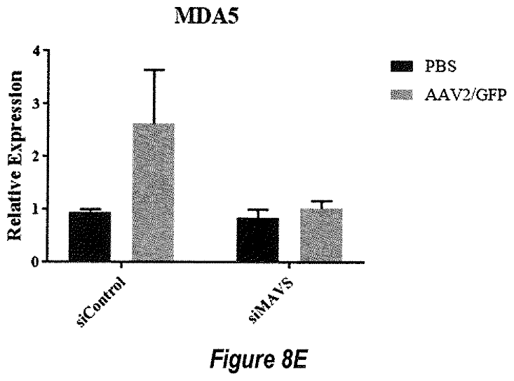

In the experiments described above, induction of the innate immune response and addition of IFN-β decreased AAV transduction. Next, we wondered whether blocking the dsRNA innate immune response impacts transgene expression at later time points after AAV transduction. Since MAD5 is a major dsRNA sensor in HeLa cells with AAV transduction, we used siRNAs specific to MDA5, and MAVS, a common adaptor for MDA5 and RIG1, to knockdown their expression, and studied the transgene expression and IFN-β expression. The transfection of siRNA was able to efficiently inhibit transcription expression of MDA5 and MAVS (FIG. 8A). At first, we examined the effect of siRNA on the inhibition of poly(I:C) on AAV transgene expression. At day 3 post AAV2/luc transduction, poly(I:C) was added. At day 4 siRNA was transfected. At 48 or 72 hrs after siRNA, the transgene expression was measured. The luciferase expression was significantly increased at both 48 and 72 hrs when siRNA to MDA5 or MAVS was used. Next, at day 4 after AAV transduction, siRNA was transfected and IFN-β expression was measured 48 and 72 hrs later. Similar to the finding from poly(I:C) application, higher luciferase expression was achieved with administration of siRNA. Finally, we studied the effect of siRNAs on IFN-β expression after AAV transduction. Consistent to studies above, high IFN-β expression was shown with siControl RNA transfection (FIG. 8B). As expected, IFN-β expression was almost completely inhibited when siRNAs to MDA5 or MAVS were used in both transcriptional and translation levels (FIG. 8C and FIG. 8D). It is interesting to note that MDA5 up-regulation was rescued when siMAVS was used (FIG. 8E). These results indicate that blockage of the dsRNA activation pathway is able to blunt the innate immune response at a late phase following AAV transduction, which leads to higher transgene expression.

The innate immune response system is the first line of defense against pathogens and its activation in AAV transduction has been studied. Compared to other pathogenic viruses, adeno-associated virus (AAV) infection or its recombinant vector transduction only induces transient and low innate immunity following AAV transduction. Also, all studies about innate immune response to virus have focused on the earlier time points after virus infection. In this study, we, for the first time, demonstrated that innate immune response was triggered at a late time point after long-term AAV transduction. The late innate immune response activation occurred in different cell lines and human primary hepatocytes. Most importantly, the late innate immune response was also detected in human hepatocytes from the liver of human xenografted mice after AAV transduction. The late innate immune response was mediated via the dsRNA activation pathway. Blocking of the dsRNA sensor or adaptor was able to blunt innate immune response and increase AAV transduction.

For innate immune response activation, generally recognition of pathogen-associated molecular patterns (PAMPs) by pattern recognition receptors (PRRs) up-regulates co-stimulatory molecules and inflammatory cytokine production. PRRs have been divided into several families: Toll-like receptors (TLRs), RIG-I like receptors (RLRs), NOD-like receptors (NLRs), C-type lectin receptors (CLRs), AIM2-like receptors (ALRs), and cytosol DNA sensors. Previous studies have demonstrated that innate immune responses are induced from AAV infection through the TLR9-MyD88 pathway in plasmacytoid DCs and TLR2 in human nonparenchymal liver cells. Another study has shown that increased TLR9 signaling was observed in the liver when AAV vectors were administered in mice via systemic administration. From these studies, the innate immune response was detected within 24 hrs.

In a further study, it was found that strong IFN-α secretion was achieved at 18 hr after pDCs were infected by AAV transduction. The activation of innate immune response was triggered in nonparenchymal liver cells (NPCs) within 24 hrs. It has been demonstrated that systemic administration of AAV vector led to rapid induction of inflammatory cytokines which returned to baseline at 6 h after AAV injection in mice. This early activation of innate immune response impacts the long-term stable transgene expression after AAV transduction. However, the early activation of innate immune response may not contribute to the declined transgene expression after week 6 in some patients with hemophilia B after AAV vector liver targeting.

In this study, up-regulation of IFN-β expression was observed at day 6 in HeLa cells after AAV transduction. In primary human hepatocytes, the pattern of IFN-β expression was inconsistent. Generally, high expression of IFN-β was achieved after day 5 in all samples. Finally, we also detected the up-regulation of IFN-β in human hepatocytes from humanized mice from week 4 to week 8 after AAV administration. These results strongly support the notion that the activation of innate immune response is elicited from long-term AAV transduction. This may inhibit later transgene expression, as manifested in clinical trials in some patients with hemophilia.

The high expression of IFN-β at day 1 of AAV transduction in some primary human hepatocytes may result from the TLR9 mediated innate immune response but not from the TLR2 pathway as suggested from early studies. The mechanism of IFN-β up-regulation at later time points after AAV transduction has not been investigated. It is unlikely that the activation of the innate immune response at the late phase is triggered by the same mechanism as that at the early phase after AAV transduction. At the early AAV infection, TLR9 recognition of the dsAAV genome or TLR2 recognition of the AAV capsid plays a major role in pDC or nonparenchymal liver cells for activation of the innate immune response, respectively. TLRs only sense PRRs localized on the cell surface or in the endosomes. After long-term AAV transduction, if PRRs from AAV vector (dsDNA AAV genome or AAV capsid protein) still remain in the endosomes, TLRs should continue to recognize these PRRs and induce a sustained IFN-β expression. This assumption is contrary to what we observed in this study that IFN-β expression was at baseline level during day 2-4 post AAV transduction in HeLa cells. Therefore, some other mechanisms should involve the activation of innate immune response at a late phase after AAV transduction. In addition to the transmembrane TLRs, cytoplasmic PRRs may also detect viral nucleic acids or proteins from virus infection. Generally, RIG-I and MDA5 are able to sense cytosolic dsRNA from RNA viruses, and several DNA sensors in cytoplasm have been identified. NLR proteins are also involved in the innate immune response to virus infection.

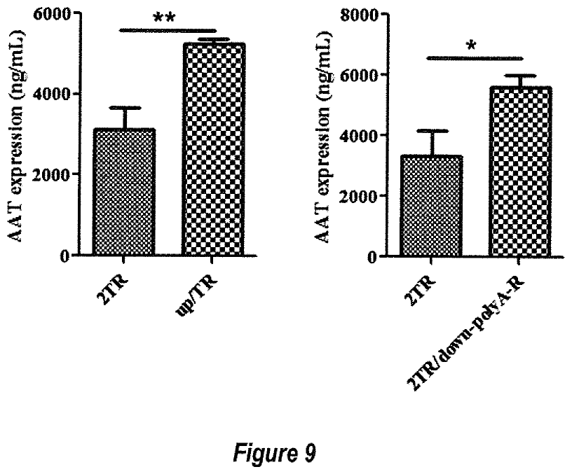

It has been demonstrated that AAV ITRs have promoter function and the 3′ITR may transcribe minus-stranded RNA, which serves as antisense to inhibit transgene expression (FIG. 9). This antisense RNA may bind to sense RNA to form dsRNA via annealing in the cytoplasm. The dsRNA generated from AAV vector transduction has potential to trigger the dsRNA innate immune response by modulation of RIG-1 and MDA5 expression, MDA-5 and RIG-1 bind to the common adaptor, MAVS, to promote direct or indirect transcriptional induction of many genes via activation of a few essential transcription factors including interferon-regulatory factors (IRFs) and NF-κB to produce IFN-β and inflammatory cytokines. Indeed, at the late time point after AAV transduction, the activation of MDA5 was observed in HeLa cells, primary human hepatocytes and hepatocytes from humanized mice.

The up-regulation of MDA5, but not RIG-1, further supports our hypothesis that dsRNA could be formed from minus stranded RNA from AAV 3′-ITR since MDA5 senses the long dsRNA. This result suggests that dsRNA mediated activation of innate immune response is triggered after long-term AAV transduction. Using siRNA to block the dsRNA sensor, MDA5, or the adaptor, MAVS, IFN-β expression was inhibited and transgene expression was increased at the late time points of AAV infection. These results further support that dsRNA activated innate immune response contributes to therapeutic FIX decrease at a later time in patients with hemophilia B receiving AAV gene therapy. As to how dsRNA mediated activation of innate immune response is only detected at later phase of AAV transduction, one of the possible mechanisms is that the promoter of AAV ITR is very weak. Therefore, it takes a relatively long time to generate enough antisense RNA from AAV 3′ITR to reach the threshold and form dsRNA. Also, the biology of AAV vector transduction may play a role in dsRNA formation at the late phase of AAV transduction. Unlike adenovirus vector, the transgene expression reaches its peak at week 6 in preclinical and clinical trials and remains persistent for long term after AAV vector administration. Therefore, a high amount of minus stranded RNA can only be synthesized at the late phase of AAV transduction.

In summary, our study reveals a novel mechanism that long term AAV transduction activates the innate immune response through the cytoplasmic dsRNA recognition pathway in transduced cells, which leads to the production of type I IFN-β. Transiently blocking the dsRNA pathway decreases IFN-β expression and increases transgene expression in AAV transduced cells. These results provide valuable information that would help us design effective approaches to interfere with dsRNA pathways for improvement of AAV transduction.

### Example 2

Block AAV ITR Promoter Function.

The exact mechanism for dsRNA induced innate immune response from AAV transduction is unknown. One of the possibilities is the promoter function of the ITR and potential bi-directional function of the promoter for transgene expression. The minus strand RNA transcribed from the 3′-ITR or the promoter, and plus strand RNA from the promoter or 5′-ITR may form double-stranded RNA which triggers an innate immune response. To prevent the transcription initiated by ITRs, we will add a poly(A) in the downstream of 5′-ITR or upstream of the promoter and 3′-ITR to block long RNA transcripts. The poly(A) can be placed as a single stretch (FIG. 10A) or in a combination (FIG. 10B) at the different locations.

Modify AAV ITR to Diminish its Promoter Function or Use an Alternative ITR without Promoter Function from a Different Serotype.

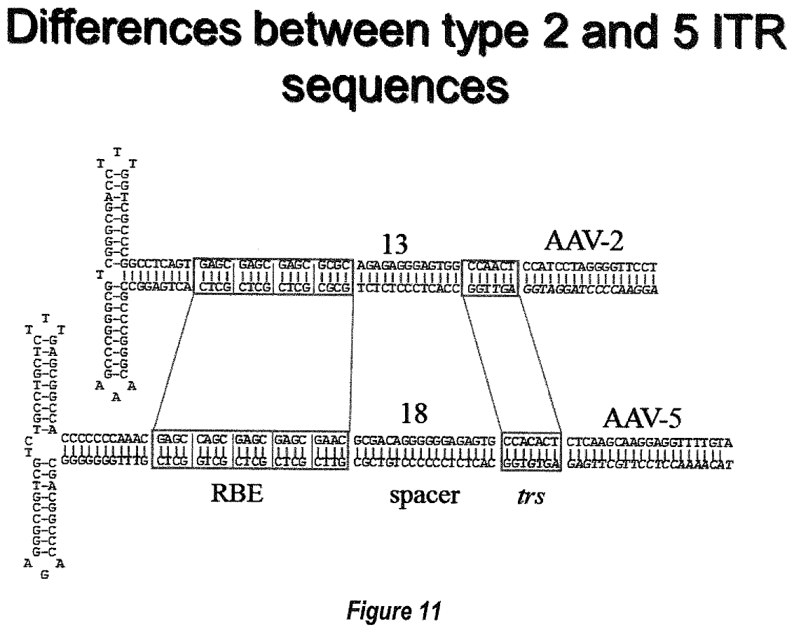

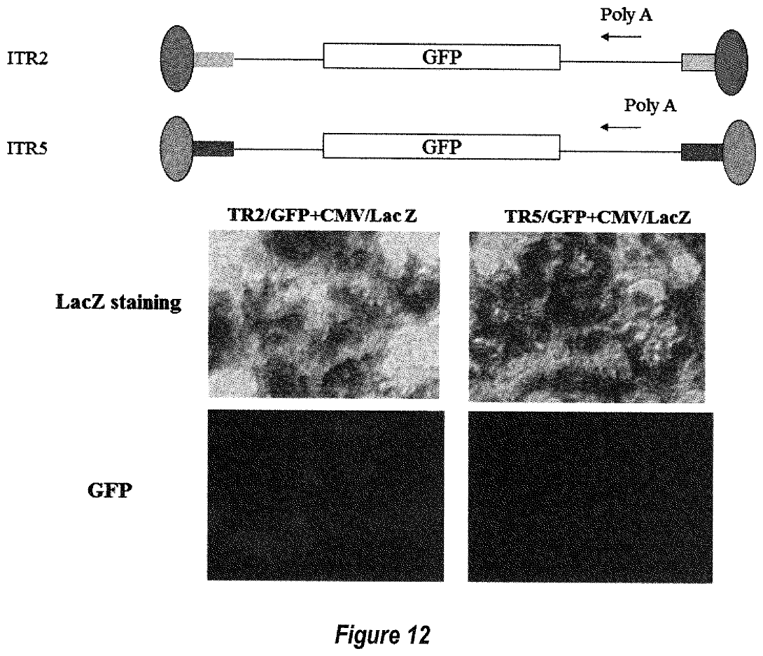

Until recently, 13 AAV serotypes and over 100 variants have been isolated. These AAV serotypes and mutants use different ITRs for virus replication and packaging. Specifically, we have studied the promoter function from AAV5 ITR since there are some differences between AAV5 ITR and AAV2 ITR (FIG. 11 and Table 7). AAV5 ITR has 5 repeats for RBEs and a longer spacer between the RBE and the trs (FIG. 11 and Table 7). To compare the promoter function from different ITRs, first we made the cassettes with different ITRs to drive GFP transgene. Following co-transfection of the plasmid with CMV/Laz as an internal control into 293 cells, two days later, 293 cells were visualized under fluorescence microscopy and stained with LacZ (FIG. 12).

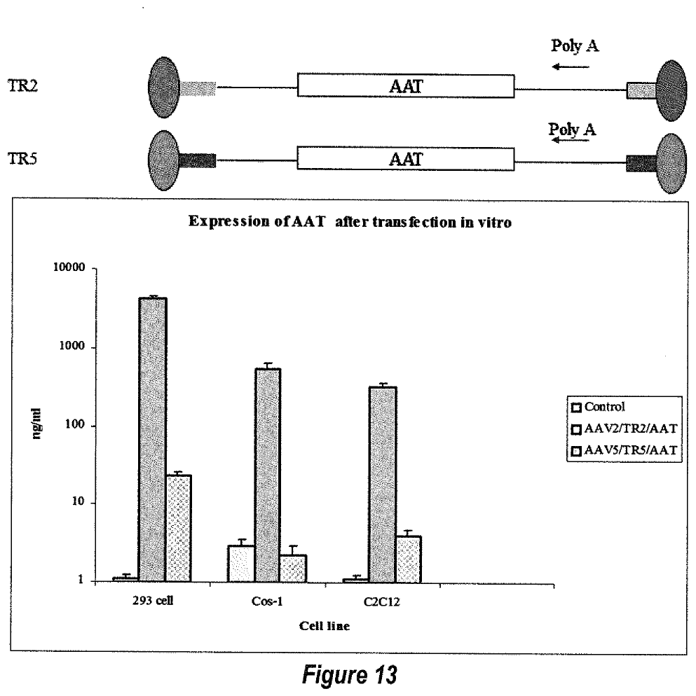

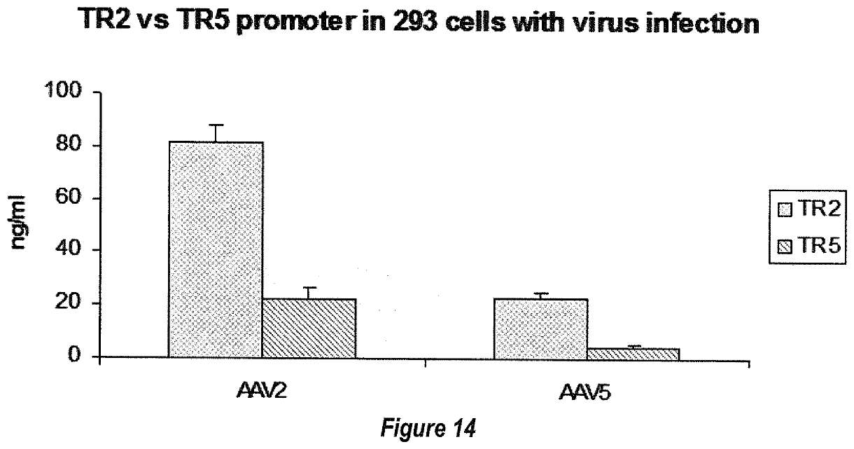

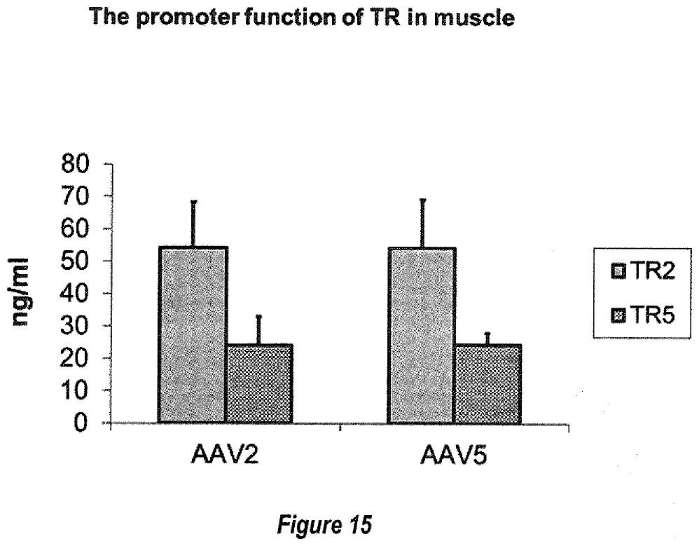

Almost no GFP positive cells were seen from transfection of ITR5/GFP when compared to that of ITR2/GFP. To further confirm the result from GFP expression, we made other cassettes using ITRs to drive human alpha-1 antitrypsin transgene (AAT). After transfection into different cells, the AAT level in the supernatant was much lower in the ITR5 cohort than that in the ITR2 cohort in all tested cell lines (FIG. 13). We packaged ITR5/AAT or ITR2/AAT into AAV2 or AAV5 capsids. After transduction of 293 cells, consistent to the result from plasmid transfection, lower AAT expression was observed from AAV/ITR5/AAT transduction regardless of different capsids (FIG. 14). After muscular injection of these vectors, AAT expression in the blood was measured at week 4 post AAV administration. Similar to in vitro transduction data, ITR5 induced much lower AAT expression than AAV2 (FIG. 15). Collectively, these results implicate that the AAV5 ITR has a weaker promoter function than that of the AAV2 ITR. It is possible that ITRs from other serotypes or variants may have no promoter function. These ITRs without promoter function will be used to generate an AAV cassette.

Knock Down the dsRNA Sensors.

MDA5 and RIG-I as well as protein kinase (PKR) are cytoplasmic dsRNA sensors. Silencing of these molecules is able to block innate immune response triggered by cytoplasmic dsRNA. The siRNA for specific sensors can be used after AAV transduction at different time points. The shRNA driven by RNA polymerase III or miRNA driven by the same RNA polymerase II for transgene expression for specific sensors can be applied using a separate vector (FIG. 16, diagram A) or as a single vector linked with a transgene cassette. When a single vector is used, shRNA or miRNA can be placed at different locations.

1. Between poly(A) and 3′ AAV ITR for shRNA (FIG. 16, diagram B)

2. Between 5′ AAV ITR and the promoter for shRNA (FIG. 16, diagram C)

3. Between the transgene and 3′ AAV ITR for miRNA (FIG. 16, diagram D)

4. Between the promoter and the transgene for miRNA (FIG. 16, diagram E)

5. Insertion of miRNA into transgene introns (FIG. 16, diagram F)

Silence the Molecules Involved in the dsRNA Innate Immune Response Activation Pathways.

Cytosolic viral RNA is recognized by receptors RIG-I and MDA5, which activate mitochondrial antiviral signaling protein (MAVS) through caspase-recruitment domain (CARD)-CARD interactions. MAVS recruits various signaling molecules to trigger downstream signaling, such as TNF receptor-associated factor 6 (TRAF6) and TRAF5. TRAF6 along with other intracellular proteins activates NF-κB signaling via receptor-interacting protein 1 (RIP1) and FAS-associated death domain protein (FADD). The NF-κB signaling phosphorylates NF-κB inhibitor-α (IκBα) and initiates pro-inflammatory cytokine gene expression. MAVS also activate interferon regulatory factor (IRF) signaling. Utilization of the same strategy as described above to knockdown MAVS or molecules involved in MAVS downstream signaling will block the dsRNA innate immune response.

MAVS signaling can also be inhibited by various molecules from virus infection. For example, a serine protease NS3-4A from hepatitis C virus, the proteases from Hepatitis A virus and GB virus B, and hepatitis B virus (HBV) X protein. During viral infection, some endogenous proteins, such as poly(rC)-binding protein 2, the 20S proteasomal subunit PSMA7, and mitofusin 2, can inhibit MAVS signaling. These proteins (inhibitors) can be expressed with a different vector (FIG. 17, diagram A) or in a single vector fused to a transgene (FIG. 17, diagram B) or driven by a different promoter (FIG. 17, diagram C) to block dsRNA immune response during therapeutic transgene expression.

Block the dsRNA Innate Immune Response Activation Pathways.

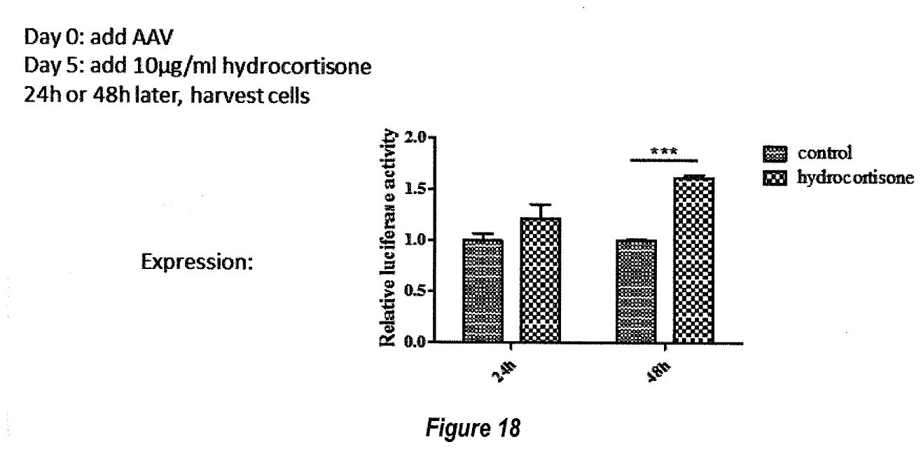

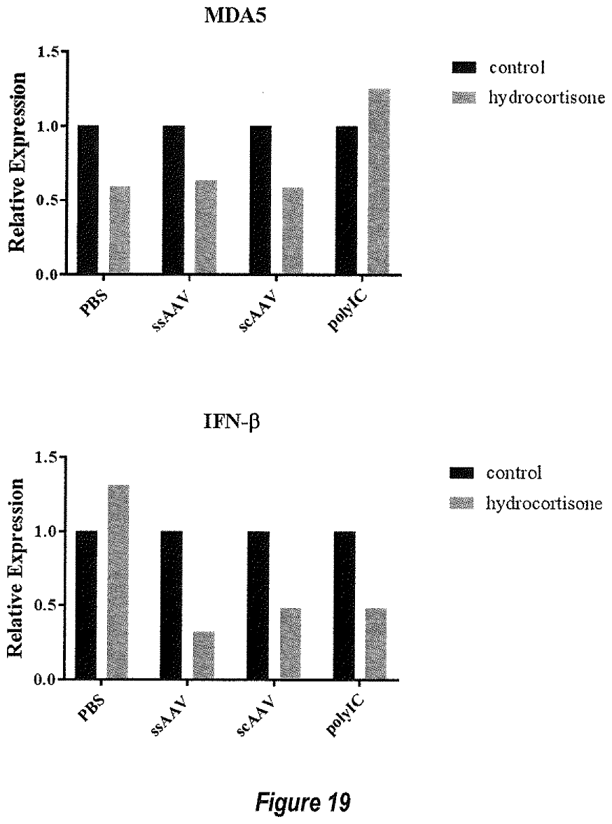

Aside from genetic approaches to block the dsRNA innate immune response, chemicals can also be used to interfere with dsRNA activation pathways. PKR is phosphorylated and activated by dsRNA and contributes to the induction of type I interferons, such as IFN-β, which can further increase its expression. 2-aminopurine (2-AP) is a potent inhibitor of double-stranded RNA (dsRNA)-activated protein kinase (PKR). Steroids such as hydrocortisone can also be used (FIGS. 18 and 19).

### Example 3

Minus Strand RNA Generation from AAV 3′ ITR after AAV Transduction.

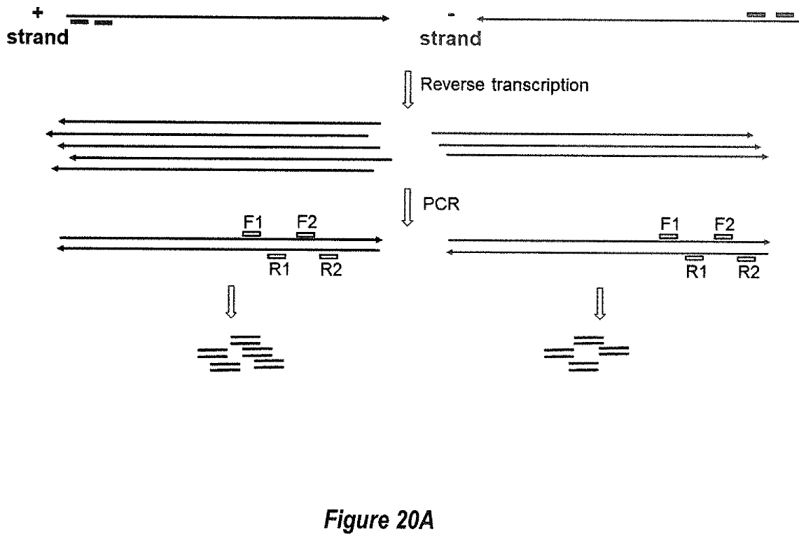

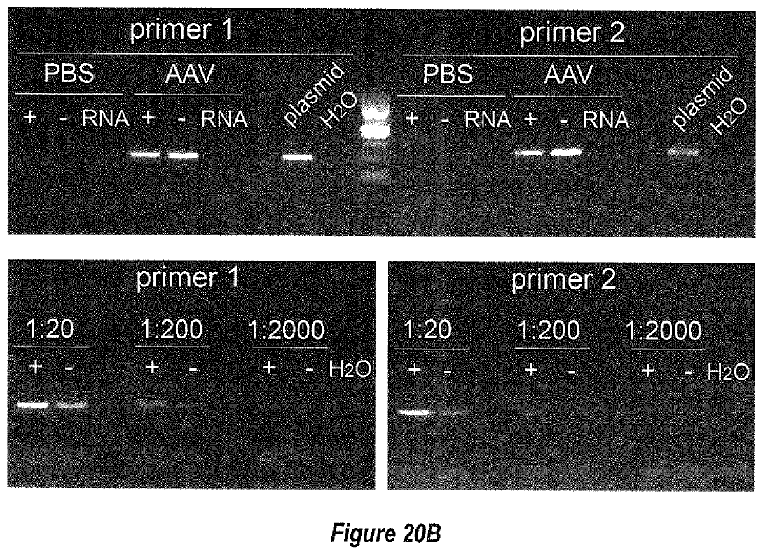

To investigate whether minus strand RNA could be generated from the AAV 3′-ITR promoter, HeLa cells were infected with AAV2/luciferase vectors and harvested 8 days later for RNA extraction. cDNA synthesis was performed using sense or antisense primers of the luciferase transgene (Table 2). Two pairs of luciferase-specific PCR primers were used to detect plus or minus strand transcripts (FIG. 20A). Both plus and minus strand transcripts were detected after AAV transduction, and there was no PCR product when RNA was used as a template (FIG. 20B). The minus strand transcripts were only detected when the cDNA template was diluted 200-fold. However, even at a 2,000-fold dilution of the cDNA template, we were still able to detect the plus strand transcript (FIG. 20B). This result indicates that the transcripts in a reverse orientation can be generated from AAV transduction and that the efficiency of minus strand RNA formation is much lower than that of plus strand RNA. It also supports the possibility that a plus strand RNA and a minus strand RNA that is generated from a different orientation are able to form dsRNA in AAV-transduced cells.

Increased AAV Transduction in Cells with MAVS Knockdown.

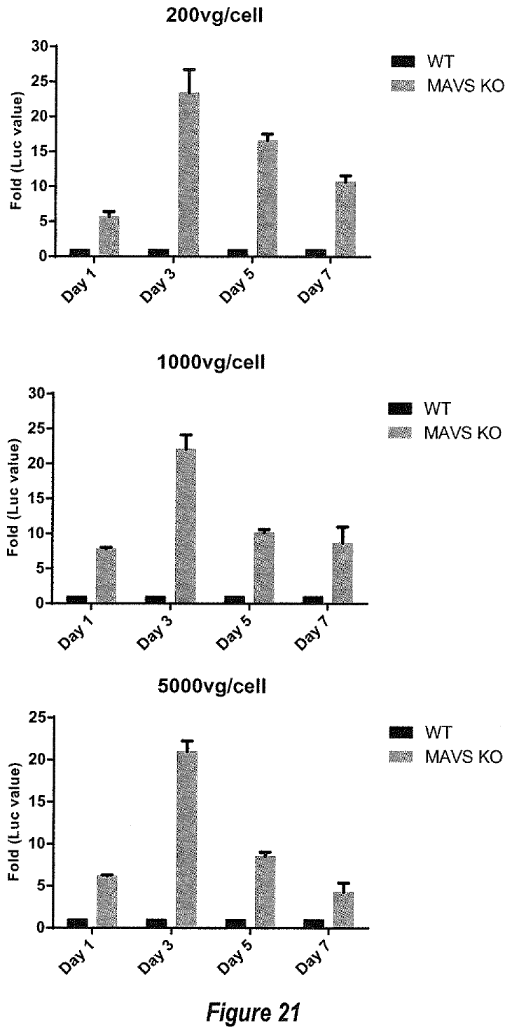

Inhibition of MAVS expression with siRNA oligo has been shown to increase AAV transduction. We examined the transduction efficiency in cells with MAVS deficiency. After transduction of AAV2/luc vectors, consistently higher transgene expression was achieved in a human hepatocyte cell line with MAVS knockdown (PH5CH8-MAVS-KO) than that in PH5CH8 cells (FIG. 21). The increased transduction was independent of vector doses and transduction duration.

Efficient Knockdown with MAVS shRNAs.

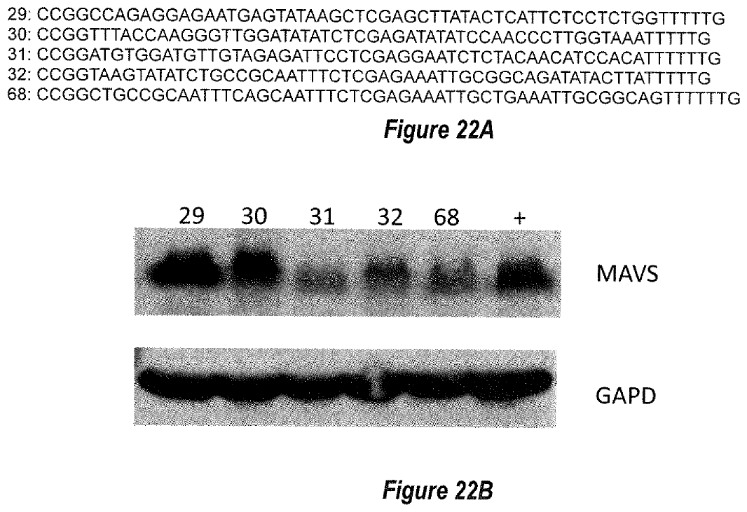

We designed 5 shRNAs driven by the U6 promoter with the potential to silence human MAVS (FIG. 22A). After transfection of shRNA plasmids into Hela cells, we examined the expression of MAVS and found that #31 induced the strongest MAVS knockdown capacity (FIG. 22B). Therefore, MAVS shRNA #31 was chosen for later studies.

Enhanced AAV Transduction in Cells with shRNA Silence of MAVS.

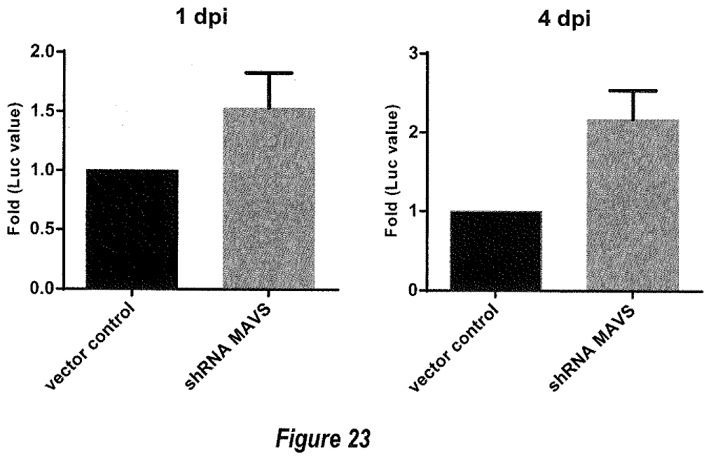

To study whether knockdown of MAVS with shRNA increases AAV transduction, we first transfected MAVS shRNA #31 into Hela cells. AAV2/luc vectors were added the next day. Transgene expression was detected at day 1 and day 4 post AAV transduction. As shown in FIG. 23, higher AAV transduction was observed in cells with MAVS silence.

In summary, increased AAV transduction is able to be achieved when the target cells are deficient for MAVS. This result indicates that integration of MAVS shRNA into AAV cassettes can induce higher AAV transduction by blocking dsRNA mediated activation of innate immune response.

While there are shown and described particular embodiments of the invention, it is to be understood that the invention is not limited thereto but may be otherwise variously embodied and practiced within the scope of the following claims. Since numerous modifications and alternative embodiments of the present invention will be readily apparent to those skilled in the art, this description is to be construed as illustrative only and is for the purpose of teaching those skilled in the art the best mode for carrying out the present invention. Accordingly, all suitable modifications and equivalents may be considered to fall within the scope of the following claims.

TABLE 1
Information about human primary hepatocyte subjects

| Subject | Age | Gender | BMI | Race | Alcohol | Tobacco | Drug |
|---|---|---|---|---|---|---|---|
| 1 | 14 | M | 22 | Caucasian | None | none | none |
| 2 | 28 | M | 25.6 | Pacific | Socially | none | none |
| | | | | Islander | | | |
| 3 | 60 | F | 25 | Caucasian | Socially | ½ ppd | none |
| 4 | 29 | M | 26.7 | African | Socially | 1 ppd before | Marijuana, |
| | | | | American | | 2013 | cocaine, and |
| | | | | | | | ecstasy socially |
| 5 | 57 | M | 33.6 | Caucasian | Socially | 1 ½ ppd | Benzodiazepines |
| 6 | 19 | M | 15.1 | Caucasian | None | none | none |
| 7 | 51 | M | 24.5 | African | 3-4 beers/ week | ½ ppd | none |
| | | | | American | 6 pack beer/day | | |
| 8 | 36 | M | 27.2 | Asian | 3 hard | 1-2 ppd | none |
| | | | | | liquor/month | | |
| 9 | 13 | M | 21 | Caucasian | None | none | none |
| 10 | 11 | M | 16.3 | Hispanic | None | none | none |
| 11 | 18 | M | 28.7 | Caucasian | None | none | none |
| 12 | 53 | M | 31.2 | African | 3-4 beers/day | 3-4 cigars/week | none |
| | | | | American | | | |

TABLE 2
Primers used in this study

| Gene name | Direction | Sequence |
|---|---|---|
| RIG-I | Forward | 5′-GGGACGAAGCAGTATTTAG-3′ |
| | | (SEQ ID NO: 6) |
| | Reverse | 5′-GGGACGAAGCAGTATTTAG-3′ |
| | | (SEQ ID NO: 7) |
| MDA-5 | Forward | 5′-CCA AAG CTG AAG AAC ACA |
| | | T-3′ (SEQ ID NO: 8) |
| | Reverse | 5′-ATC TTC TCT GGT TGC ATC |
| | | T-3′ (SEQ ID NO: 9) |
| IFN-β | Forward | 5′-CAGCAATTTTCAGTGTCAGAAGC |
| | | T-3′ (SEQ ID NO: 10) |
| | Reverse | 5′-TCATCCTGTCCTTGAGGCAGT-3′ |
| | | (SEQ ID NO: 11) |
| GAPDH | Forward | 5′-GCACCGTCAAGGCTGAGAAC-3′ |
| | | (SEQ ID NO: 12) |
| | Reverse | 5′-ATGGTGGTGAAGACGCCAG-3′ |
| | | (SEQ ID NO: 13) |
| Luciferase | Forward | 5′-CGCTGGGCGTTAATCAAAGA-3′ |
| | | (SEQ ID NO: 14) |
| | Reverse | 5′-AGCCACCTGATAGCCTTTGT-3′ |
| | | (SEQ ID NO: 15) |
| Luciferase | Forward | 5′-ACTGGGACGAAGACGAACAC- |
| | | 3′(SEQ ID NO: 16) |
| | Reverse | 5′-GGCGACGTAATCCACGATCT-3′ |
| | | (SEQ ID NO: 17) |
| Luciferase | Forward | 5′-AGAGATACGCCCTGGTTCCT-3′ |
| for reverse | | (SEQ ID NO: 18) |
| transcription | Forward | 5′-CCTACCGTGGTGTTCGTTTC-3′ |
| | | (SEQ ID NO: 19) |
| | Forward | 5′-TTGTGCCAGAGTCCTTCGAT-3′ |
| | | (SEQ ID NO: 20) |
| | Forward | 5′-AAGCGGTTGCCAAGAGGTTC-3′ |
| | | (SEQ ID NO: 21) |
| | Forward | 5′-ATTACACCCGAGGGGGATGA-3′ |
| | | (SEQ ID NO: 22) |
| | Forward | 5′-CGCTGGGCGTTAATCAAAGA-3′ |
| | | (SEQ ID NO: 23) |
| | Forward | 5′-ACTGGGACGAAGACGAACAC-3′ |
| | | (SEQ ID NO: 24) |
| | Forward | 5′-CGCCAGTCAAGTAACAACCG-3′ |
| | | (SEQ ID NO: 25) |
| | Reverse | 5′-GCTGCGAAATGCCCATACTG-3′ |
| | | (SEQ ID NO: 26) |
| | Reverse | 5′-ATCGAAGGACTCTGGCACAA-3′ |
| | | (SEQ ID NO: 27) |
| | Reverse | 5′-ATCTCACGCAGGCAGTTCTA-3′ |
| | | (SEQ ID NO: 28) |
| | Reverse | 5′-CGCGCCCGGTTTATCATC-3′ |
| | | (SEQ ID NO: 29) |
| | Reverse | 5′-TCTCACACACAGTTCGCCTC-3′ |
| | | (SEQ ID NO: 30) |
| | Reverse | 5′-AGCCACCTGATAGCCTTTGT-3′ |
| | | (SEQ ID NO: 31) |
| | Reverse | 5′-GGCGACGTAATCCACGATCT-3′ |
| | | (SEQ ID NO: 32) |
| | Reverse | 5′-CGATCTTTCCGCCCTTCTTG-3′ |
| | | (SEQ ID NO: 33) |

TABLE 3

| | GenBank Accession |
|---|---|
| | Number |
| Complete Genomes | |
| Adeno-associated virus 1 | NC_002077, AF063497 |
| Adeno-associated virus 2 | NC_001401 |
| Adeno-associated virus 3 | NC_001729 |
| Adeno-associated virus 3B | NC_001863 |
| Adeno-associated virus 4 | NC_001829 |
| Adeno-associated virus 5 | Y18065, AF085716 |
| Adeno-associated virus 6 | NC_001862 |
| Avian AAV ATCC VR-865 | AY186198, AY629583, |
| | NC_004828 |
| Avian AAV strain DA-1 | NC_006263, AY629583 |
| Bovine AAV | NC_005889, |
| | Ay388617, AAR26465 |
| AAV11 | AAT46339, AY631966 |
| AAV12 | ABI16639, DQ813647 |
| Clade A | |
| AAV1 | NC_002077, AF063497 |
| AAV6 | NC_001862 |
| Hu.48 | AY530611 |
| Hu 43 | AY530606 |
| Hu 44 | AY530607 |
| Hu 46 | AY530609 |
| Clade B | |
| Hu. 19 | AY530584 |
| Hu. 20 | AY530586 |
| Hu 23 | AY530589 |
| Hu22 | AY530588 |
| Hu24 | AY530590 |
| Hu21 | AY530587 |
| Hu27 | AY530592 |
| Hu28 | AY530593 |
| Hu 29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| Hu14 (AAV9) | AY530579 |
| Hu31 | AY530596 |
| Hu32 | AY530597 |
| Clonal Isolate | |
| AAV5 | Y18065, AF085716 |
| AAV 3 | NC_001729 |
| AAV 3B | NC_001863 |
| AAV4 | NC_001829 |
| Rh34 | AY243001 |
| Rh33 | AY243002 |
| Rh32 | AY243003 |

TABLE 4
Amino acid residues and abbreviations

| | Abbreviation | |
|---|---|---|
| Amino Acid Residue | Three-Letter Code | One-Letter Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 5

| Serotype | Position 1 | Position 2 |
|---|---|---|
| AAV1 | A263X | T265X |
| AAV2 | Q263X | −265X |
| AAV3a | Q263X | −265X |
| AAV3b | Q263X | −265X |
| AAV4 | S257X | −259X |
| AAV5 | G253X | V255X |
| AAV6 | A263X | T265X |
| AAV7 | E264X | A266X |
| AAV8 | G264X | S266X |
| AAV9 | S263X | S265X |

Where (X) → mutation to any amino acid (−) → insertion of any amino acid
Note:
Position 2 inserts are indicated by site of insertion

TABLE 6

| | |
|---|---|
| Modified Amino Acid Residue | Abbreviation |

Amino Acid Residue Derivatives

| | |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | aIle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-Cl) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

TABLE 7
Comparison of TR2 and TR5

| | TR2 | TR5 |
|---|---|---|
| Homology | 58% | 58% |
| RBS GAGY motif (n) | 4 | 5 |
| TRS sequence | CCAACT | CCACACT |
| | (SEQ ID NO: 34) | (SEQ ID NO: 35) |
| The space from | 13 | 18 |
| RBS to TRS | | |
| Rep2 binding | + | + |
| Rep5 binding | + | + |
| Rep2 nicking | + | − |
| Rep5 nicking | − | + |
| Specific integration | Chromosome 19 | ? |

1. A recombinant adeno-associated virus (rAAV) vector genome designed to reduce the generation of double stranded RNA in AAV vector transduction and/or to inhibit an innate immune response that may result from AAV vector transduction, comprising an adeno-associated virus (AAV) 5′ inverted terminal repeat (ITR), a nucleotide sequence of interest (NOI) operably associated with a promoter and an AAV 3′ ITR, and further comprising:
A) one or more poly A (pA) sequences selected from:
a) a poly A (pA) sequence downstream of the 5′ ITR and upstream of the promoter, in 3′ to 5′ orientation and a pA sequence upstream of the 3′ ITR and downstream of the NOI, in 3′ to 5′ orientation;
b) a pA sequence upstream of the 3′ ITR and downstream of the NOI, in 3′ to 5′ orientation;
c) a first pA sequence upstream of the 3′ ITR and downstream of the NOI, in 3′ to 5′ orientation and a second pA sequence downstream of the first pA sequence and upstream of the 3′ ITR, in a 5′ to 3′ orientation;
d) a first pA sequence upstream of the 3′ ITR and downstream of the NOI, in 3′ to 5′ orientation and a second pA sequence downstream of the NOI and upstream of the first pA, in a 5′ to 3′ orientation;
e) a first pA sequence upstream of the 3′ ITR and downstream of the NOI, in 3′ to 5′ orientation and a second pA sequence downstream of the 5′ ITR and upstream of the promoter, in a 5′ to 3′ orientation;
f) a first pA sequence downstream of the 5′ ITR and upstream of the promoter, in 3′ to 5′ orientation, a second pA sequence downstream of the NOI and upstream of a third pA sequence, in 5′ to 3′ orientation and the third pA sequence downstream of the second pA sequence and upstream of the 3′ ITR, in 3′ to 5′ orientation;
g) a first pA sequence downstream of the 5′ ITR and upstream of the promoter, in 5′ to 3′ orientation, a second pA sequence downstream of the NOI and upstream of a third pA sequence, in 3′ to 5′ orientation and the third pA sequence downstream of the second pA sequence and upstream of the 3′ ITR, in 5′ to 3′ orientation;
h) a first pA sequence downstream of the 5′ ITR and upstream of the promoter, in 5′ to 3′ orientation, a second pA sequence downstream of the NOI and upstream of a third pA sequence, in 5′ to 3′ orientation and the third pA sequence downstream of the second pA sequence and upstream of the 3′ ITR, in 3′ to 5′ orientation;
i) a first pA sequence downstream of the 5′ ITR and upstream of the promoter, in 5′ to 3′ orientation, a second pA sequence downstream of the NOI and upstream of a third pA sequence, in 3′ to 5′ orientation and the third pA sequence downstream of the second pA sequence and upstream of the 3′ ITR, in 5′ to 3′ orientation;
j) a first pA sequence downstream of the 5′ ITR and upstream of a second pA sequence, in 3′ to 5′ orientation, the second pA sequence downstream of the first pA sequence and upstream of the promoter, in 5′ to 3′ orientation; a third pA sequence downstream of the NOI and upstream of a fourth pA sequence, in 5′ to 3′ orientation and the fourth pA sequence downstream of the third pA sequence and upstream of the 3′ ITR, in 3′ to 5′ orientation;
k) a first pA sequence downstream of the 5′ ITR and upstream of a second pA sequence, in 3′ to 5′ orientation, the second pA sequence downstream of the first pA sequence and upstream of the promoter, in 5′ to 3′ orientation; a third pA sequence downstream of the NOI and upstream of a fourth pA sequence, in 3′ to 5′ orientation and the fourth pA sequence downstream of the third pA sequence and upstream of the 3′ ITR, in 5′ to 3′ orientation;
l) a first pA sequence downstream of the 5′ ITR and upstream of a second pA sequence, in 5′ to 3′ orientation, the second pA sequence downstream of the first pA sequence and upstream of the promoter, in 3′ to 5′ orientation; a third pA sequence downstream of the NOI and upstream of a fourth pA sequence, in 5′ to 3′ orientation and the fourth pA sequence downstream of the third pA sequence and upstream of the 3′ ITR, in 3′ to 5′ orientation; and/or
m) a first pA sequence downstream of the 5′ ITR and upstream of a second pA sequence, in 5′ to 3′ orientation, the second pA sequence downstream of the first pA sequence and upstream of the promoter, in 3′ to 5′ orientation; a third pA sequence downstream of the NOI and upstream of a fourth pA sequence, in 3′ to 5′ orientation and the fourth pA sequence downstream of the third pA sequence and upstream of the 3′ ITR, in 5′ to 3′ orientation;
B) one or more nucleic acid molecules that encode an interfering RNA (RNAi) that targets a cytoplasmic dsRNA sensor; and/or
C) a nucleic acid molecule that encodes an inhibitor of MAVS signaling.

2-3. (canceled)

4. The rAAV vector genome of claim 1, wherein the 5′ ITR and/or the 3′ ITR is modified to diminish or eliminate promoter activity from the 5′ ITR and/or the 3′ ITR.

5. The rAAV vector genome of claim 1, wherein the NOI sequence is fused with the one or more nucleic acid molecules of B) and/or the nucleic acid molecule of C).

6. The rAAV vector genome of claim 1, wherein the one or more nucleic acid molecules of B) are operably associated with a second promoter.

7. The rAAV vector genome of claim 1, wherein the RNAi is a small interfering RNA (siRNA), a short hairpin RNA (shRNA), a microRNA (miRNA), a long double stranded RNA (long dsRNA), an antisense RNA, or a ribozyme.

8. The rAAV vector genome of claim 7, wherein the vector comprises an AAV 5′ ITR, a shRNA operably associated with a first promoter, a NOI operably associated with a second promoter, a pA sequence in 3′ to 5′ orientation, and an AAV 3′ ITR.

9. The rAAV vector genome of claim 7, comprising in the following order: an AAV 5′ ITR, a NOI and a miRNA both operably associated with a promoter, a pA sequence in 3′ to 5′ orientation, and an AAV 3′ ITR.

10. The rAAV vector genome of claim 7, comprising in the following order: an AAV 5′ ITR, a miRNA and a NOI both operably associated with a promoter, a pA sequence in 3′ to 5′ orientation, and an AAV 3′ ITR.

11. The rAAV vector genome of claim 7, comprising in the following order: an AAV 5′ ITR, a NOI comprising a miRNA intron sequence within the NOI, the NOI being operably associated with a promoter, a pA sequence in 3′ to 5′ orientation, and an AAV 3′ ITR.

12-14. (canceled)

15. The rAAV vector genome of claim 1, wherein the inhibitor of MAVS signaling is selected from the group consisting of: a serine protease NS3-4A from hepatitis C virus, a protease from Hepatitis A virus, a protease from GB virus B, hepatitis B virus (HBV) X protein, poly(rC)-binding protein 2, the 20S proteasomal subunit PSMA7, mitofusin 2, and any combination thereof.

16-17. (canceled)

18. The rAAV vector genome of claim 1 that is comprised within a rAAV particle.

19. A composition comprising the rAAV vector genome of claim 18.

20. The composition of claim 19, further comprising a recombinant nucleic acid molecule that encodes an interfering RNA sequence that targets a cytoplasmic dsRNA sensor and/or a recombinant nucleic acid molecule that encodes an inhibitor of MAVS signaling.

21. A method of enhancing transduction of an AAV vector in cells of a subject, comprising administering to the subject an AAV vector and an agent that interferes with dsRNA activation pathways in cells of the subject.

22. The method of claim 21, wherein the agent that interferes with dsRNA activation pathways in cells of the subject is 2-aminopurine.

23. The method of claim 21, wherein the AAV vector and the agent are administered to the subject simultaneously.

24. The method of claim 21, wherein the AAV vector and the agent are administered at separate times.

25. The rAAV vector genome of claim 1, that is comprised within a plasmid.

26. The rAAV vector genome of claim 25, that is comprised within a cell.

27. The rAAV particle of claim 18, that is of a first AAV serotype, wherein the AAV 5′ ITR and/or the AAV 3′ ITR is from a second AAV serotype that is different than the first AAV serotype.

28. The rAAV particle of claim 27, wherein the first AAV serotype is AAV2 and the second AAV serotype is AAV5.


SEQUENCE LISTING (40 sequences)

SEQ ID NO: 1 - RNA, 19 nt, Artificial
  siMDA5
cugaaucugcuccuucacc

SEQ ID NO: 2 - RNA, 19 nt, Artificial
  siMAVS
auacaacugacccuguggg

SEQ ID NO: 3 - RNA, 19 nt, Artificial
  siMAVS-2
uaguugaucucgcggacga

SEQ ID NO: 4 - RNA, 19 nt, Artificial
  siMAVS-2
ccguuugcugaagacaaga

SEQ ID NO: 5 - RNA, 19 nt, Artificial
  siControl
ugugaucaaggacgcuaug

SEQ ID NO: 6 - DNA, 19 bp, Artificial
  primer
gggacgaagcagtatttag

SEQ ID NO: 7 - DNA, 19 bp, Artificial
  primer
gggacgaagcagtatttag

SEQ ID NO: 8 - DNA, 19 bp, Artificial
  primer
ccaaagctgaagaacacat

SEQ ID NO: 9 - DNA, 19 bp, Artificial
  primer
atcttctctggttgcatct

SEQ ID NO: 10 - DNA, 24 bp, Artificial
  primer
cagcaattttcagtgtcagaagct

SEQ ID NO: 11 - DNA, 21 bp, Artificial
  primer
tcatcctgtccttgaggcagt

SEQ ID NO: 12 - DNA, 20 bp, Artificial
  primer
gcaccgtcaaggctgagaac

SEQ ID NO: 13 - DNA, 19 bp, Artificial
  primer
atggtggtgaagacgccag

SEQ ID NO: 14 - DNA, 20 bp, Artificial
  primer
cgctgggcgttaatcaaaga

SEQ ID NO: 15 - DNA, 20 bp, Artificial
  primer
agccacctgatagcctttgt

SEQ ID NO: 16 - DNA, 20 bp, Artificial
  primer
actgggacgaagacgaacac

SEQ ID NO: 17 - DNA, 20 bp, Artificial
  primer
ggcgacgtaatccacgatct

SEQ ID NO: 18 - DNA, 20 bp, Artificial
  primer
agagatacgccctggttcct

SEQ ID NO: 19 - DNA, 20 bp, Artificial
  primer
cctaccgtggtgttcgtttc

SEQ ID NO: 20 - DNA, 20 bp, Artificial
  primer
ttgtgccagagtccttcgat

SEQ ID NO: 21 - DNA, 20 bp, Artificial
  primer
aagcggttgccaagaggttc

SEQ ID NO: 22 - DNA, 20 bp, Artificial
  primer
attacacccgagggggatga

SEQ ID NO: 23 - DNA, 20 bp, Artificial
  primer
cgctgggcgttaatcaaaga

SEQ ID NO: 24 - DNA, 20 bp, Artificial
  primer
actgggacgaagacgaacac

SEQ ID NO: 25 - DNA, 20 bp, Artificial
  primer
cgccagtcaagtaacaaccg

SEQ ID NO: 26 - DNA, 20 bp, Artificial
  primer
gctgcgaaatgcccatactg

SEQ ID NO: 27 - DNA, 20 bp, Artificial
  primer
atcgaaggactctggcacaa

SEQ ID NO: 28 - DNA, 20 bp, Artificial
  primer
atctcacgcaggcagttcta

SEQ ID NO: 29 - DNA, 18 bp, Artificial
  primer
cgcgcccggtttatcatc

SEQ ID NO: 30 - DNA, 20 bp, Artificial
  primer
tctcacacacagttcgcctc

SEQ ID NO: 31 - DNA, 20 bp, Artificial
  primer
agccacctgatagcctttgt

SEQ ID NO: 32 - DNA, 20 bp, Artificial
  primer
ggcgacgtaatccacgatct

SEQ ID NO: 33 - DNA, 20 bp, Artificial
  primer
cgatctttccgcccttcttg

SEQ ID NO: 34 - DNA, 6 bp, Artificial
  TRS sequence
ccaact

SEQ ID NO: 35 - DNA, 7 bp, Artificial
  TRS sequence
ccacact

SEQ ID NO: 36 - DNA, 58 bp, Artificial
  shMAVS #29
ccggccagaggagaatgagtataagctcgagcttatactcattctcctctggtttttg

SEQ ID NO: 37 - DNA, 58 bp, Artificial
  shMAVS #30
ccggtttaccaagggttggatatatctcgagatatatccaacccttggtaaatttttg

SEQ ID NO: 38 - DNA, 58 bp, Artificial
  shMAVS #31
ccggatgtggatgttgtagagattcctcgaggaatctctacaacatccacattttttg

SEQ ID NO: 39 - DNA, 58 bp, Artificial
  shMAVS #32
ccggtaagtatatctgccgcaatttctcgagaaattgcggcagatatacttatttttg

SEQ ID NO: 40 - DNA, 59 bp, Artificial
  shMAVS #68
ccggctgccgcaatttcagcaatttctcgagaaattgctgaaattgcggcagttttttg